(19) United States
(12) Patent Application Publication
(10) Pub. No.: US 2023/0165976 A1
(43) Pub. Date: Jun. 1, 2023

(54) HTRA1 MODULATION FOR TREATMENT OF AMD

(71) Applicant: University of Utah Research Foundation, Salat Lake City, UT (US)

(72) Inventors: Brandi Williams, Holladay, UT (US); Gregory Hageman, Bountiful, UT (US); Burt Timothy Richards, Midway, UT (US); Osama Youssef, Sall Lake City, UT (US)

(21) Appl. No.: 17/599,979

(22) PCT Filed: Apr. 10, 2020
(86) PCT No.: PCT/US2020/027802

§ 371 Date: Sep. 29, 2021

Related U.S. Application Data
(60) Provisional application No. 62/832,182, filed on Apr. 10, 2019.

(51) Int. Cl. A61K 48/00; C12N 15/11; A61K 31/7088; C12N 9/22

(52) U.S. Cl. CPC A61K 48/0066; C12N 15/11; A61K 31/7088; C12N 9/22; C12N 2310/20

(57) ABSTRACT

The invention provides compositions and methods for treatment of Chromosome 10-driven age-related macular degeneration, including gene therapy to increase HTRA1 expression in retinal pigmented epithelial cells in the eye.

### FIELD OF THE INVENTION

The invention relates to methods and compositions for treatment of age-related macular degeneration, and finds application in the fields of biology and medicine.

### SEQUENCE LISTING

The instant application contains a Sequence Listing which has been submitted electronically in ASCII format and is hereby incorporated by reference in its entirety. Said ASCII copy, created on Apr. 9, 2020, is named 098846-1185529-000810PC_SL.txt and is 46,642 bytes in size.

### CROSS-REFERENCE TO RELATED APPLICATIONS

This application is a national phase application of International Application No. PCT/US2020/027802, filed Apr. 10, 2020, which claims benefit of U.S. provisional application No. 62/832,182, filed Apr. 10, 2019, the entire content of which is incorporated herein by reference.

### BACKGROUND OF THE INVENTION

Age-related macular degeneration (AMD) is the leading cause of irreversible vision loss in the developed world (for reviews see Zarbin, Eur. Ophthalmol. 8:199-206, (1998) affecting approximately 15% of individuals over the age of 60. An estimated 600 million individuals are in this age demographic. The prevalence of AMD increases with age; mild, or early forms occur in nearly 30%, and advanced forms in about 7%, of the population that is 75 years and older (Vingerling et al., Epidemiol. Rev. 17(2):347-360, 1995).

Traditional therapies generally involve intravitreal injection of a therapeutic agent, e.g., an anti-VEGF agent, on a bi-monthly or as needed basis. These therapies require repeated, invasive surgery, which has caused significant discomfort and inconvenience to patients. The procedures involved in these therapies may also increase the risk of side effects. Further, in most cases, the therapeutic agents are administered only after the choroidal neovascularization (CNV) has developed and when there is significant damage to the macula. There are no therapies currently approved for early stage AMD or for preventing the development of AMD.

Based on extensive genotyping studies of AMD patients it is now understood that AMD includes two distinct biological diseases: Chromosome 1-directed AMD (or “Chr 1 AMD”), which results from dysregulation of the complement system, including complement factor H dysregulation, and chromosome 10-directed AMD (or “Chr 10 AMD”), which is associated with genetic variants in chromosomal region 10q26, which harbors the ARMS2 and HTRA1 genes. See Keenan et al, 2015, “ASSESSMENT OF PROTEINS ASSOCIATED WITH COMPLEMENT ACTIVATION AND INFLAMMATION IN MACULAE OF HUMAN DONORS HOMOZYGOUS RISK AT CHROMOSOME 1 CFH-TO-F13B” Invest Ophthalmol Vis Sci. 56:487-79; Hageman, 2015, “METHODS OF PREDICTING THE DEVELOPMENT OF AMD BASED ON CHROMOSOME 1 AND CHROMOSOME 10” US Pat. Pub. 2015/0211065, incorporated herein by reference. It has been reported that these loci on chromosomes 1 and 10 together account for 95% of all AMD risk in Caucasian population and that chromosomal region 10q26 is strongly associated with increased risk of AMD (Fisher et al., 2005; Rivera et. al. 2005). The risk region comprises genes (including ARMS2 and HTRA1) that share extensive linkage disequilibrium (LD) (D′=0.99).

### BRIEF SUMMARY OF THE INVENTION

Methods and compositions for treating, preventing development of, slowing progression of, reversing or ameliorating symptoms and signs of chromosome 10-driven age-related macular degeneration (AMD) are provided in this disclosure.

In some aspects, the disclosure provides a method of treating, preventing the development of, slowing progression of, reversing or ameliorating the symptoms and signs of Chr 10 AMD in a subject, by administering an agent that increases HTRA1 expression in cells of the subject (e.g., RPE cells, horizontal cells, or photoreceptor cells). In some cases, the subject has a single chromosome 10 risk allele. In some cases, the subject is homozygous for the chromosome 10 risk allele. In some cases, the subject exhibits a Chr 10 AMD clinical phenotype. In some cases, the subject does not carry a chromosome 1 risk allele(s).

In some approaches, the agent causes upregulation of transcription of an endogenous HTRA1 gene sequence. For example, the agent may be a transcriptional activator that binds a target sequence within an HTRA1 transcriptional regulatory region or an HTRA1 promoter. In some cases, the agent is a fusion protein of i) a DNA targeting protein capable of recognizing a target sequence within the HTRA1 transcriptional regulatory region, and ii) the transcriptional activator. In some cases, the DNA targeting protein recognizes the target sequence through a guide RNA that is complementary to a sequence within the HTRA1 transcriptional regulation region or the HTRA1 promoter region. In some cases, the DNA targeting protein is an enzymatically inactive Cas9 protein (dCas9). In some cases, the transcriptional activator is VP16. In some cases, the method further comprises administering a sgRNA that is complementary to a sequence within the HTRA1 transcriptional regulation region.

In some cases, administration of the agent results in expression of an exogenous HTRA1 protein in cells of the subject (including, e.g., RPE cells, horizontal cells, or photoreceptor cells). In a gene therapy approach the agent may be a vector, such as a viral vector (e.g., AAV or lentivirus), that delivers an exogenous HTRA1 protein or a nucleic acid sequence encoding HTRA1 protein, preferably operably linked to a promoter. In some cases, the promoter is a RPE-specific promoter.

In some cases, treating a subject using the agent results in modification of genomic DNA in RPE cells of the subject. For example, genome editing methods may be used to change a sequence in the HTRA1 gene or transcription regulatory regions and convert one or more risk alleles to corresponding non-risk allele(s). In some cases, the agent is a DNA endonuclease-based system. In some cases, the DNA endonuclease is a Cas9 endonuclease, a Zinc-finger nuclease, a transcription activator-like effector nuclease, a Homing endonuclease, a Meganuclease or a Cre recombinase. In some cases, the agent modifies the genomic DNA and results in a genomic DNA sequence comprising SEQ ID NO: 34. In some cases, the agent modifies the genomic DNA and results in a genomic DNA sequence comprising SEQ ID NO: 14. In some cases, a risk allele sequence (c) at rs36212733 is changed to a non-risk allele sequence (t). In some cases, the agent is administered by subretinal injection. In some cases the agent is delivered by suprachoroidal injection. In some cases the agent is delivered by intravitreal injection. In some cases the agent is delivered by other routes. In some cases, the one or more agents comprise Cas9 and one or more sgRNAs, wherein the one or more sgRNAs direct the cas9 to a genomic region comprising the one or more risk alleles, thereby modifying the genomic region, which results in the one or more risk alleles being replaced with corresponding non-risk alleles.

In some cases, the agent is a small molecule compound, a peptide, or a nucleic acid that increases HTRA1 expression or activity in RPE cells.

In some aspects, the disclosure provides a pharmaceutical composition for treating Chr 10 AMD, comprising (i) an agent that increases HTRA1 expression in RPE cells of the subject, and (ii) a pharmaceutically acceptable carrier.

Compositions of the invention in one form include a guide RNA (gRNA) comprising a guide sequence of at least 10 contiguous nucleotides corresponding to a target sequence in the HTRA1 promoter or in the HTRA1 2 kb regulatory region. In one aspect, HTRA1 promoter has the sequence set forth in SEQ ID NO: 5, 7, 8, or 13 and the 2 kb regulatory region has the sequence set forth in SEQ ID NO: 14.

In another aspect, the invention includes a ribonucleoprotein (RNP) complex comprising: (a) guide RNA (gRNA) comprising a guide sequence of at least 10 contiguous nucleotides corresponding to a target sequence in the HTRA1 promoter or in the HTRA1 2 kb regulatory region; and (b) a fusion protein comprising a CRISPR-associated protein (Cas) domain fused to a transcriptional activator domain, wherein the Cas protein domain lacks nuclease activity and the HTRA1 promoter has the sequence set forth in SEQ ID NO: 5, 7, 8, or 13, and the 2 kb regulatory region has the sequence set forth in SEQ ID NO: 14. In one embodiment, the Cas of the RNP complex is dCas9 or dCas12a. In one aspect, the target sequence of the RNP complex is in the promoter and the transcriptional activator is selected from VP16, VP64, VP160, MLL, E2A, HSF1, NF-IL6, NFAT1 and NF-kB. In another aspect, the target sequence of the RNP complex is in the 2 kb regulatory region and the transcriptional activator is LHX2.

In a further aspect, the invention includes a ribonucleoprotein (RNP) complex comprising: (a) guide RNA (gRNA) comprising a guide sequence of at least 10 contiguous nucleotides corresponding to a target sequence in the HTRA1 2 kb regulatory region; and (b) a CRISPR-associated (Cas) protein, wherein the 2 kb regulatory region has the sequence set forth in SEQ ID NO: 14. In one embodiment, the Cas of the RNP complex is Cas is Cas9, Cas12a, or Cas3.

In one aspect, the guide sequence of the gRNA or RNP complex comprises at least 15 contiguous nucleotides corresponding to the target sequence. In another aspect the guide sequence of the gRNA or RNP complex comprises at least 20 contiguous nucleotides corresponding to the target sequence. In yet another aspect, the guide sequence of the gRNA or RNP complex comprises 15-25 contiguous nucleotides corresponding to the target sequence. In one embodiment, target sequence of the gRNA or RNP complex is contiguous with a protospacer adjacent motif (PAM) is NGG.

In one aspect, the guide sequence of the gRNA or the RNP complex comprises any of SEQ ID NOS: 15-33, or a subsequence comprising at least 15 contiguous bases of any of SEQ ID NOS: 15-33.

In another aspect, the invention includes gRNA or the RNP complex, wherein (i) the guide sequence comprises any of SEQ ID NOS: 36-49; and/or (ii) target sequence comprises or is adjacent to a risk allele selected from risk at rs10490924, rs144224550, rs36212731, rs36212732, rs36212733, rs3750848, rs3750847, and rs3750846.

In another aspect, the invention includes a polynucleotide encoding the gRNA, wherein the polynucleotide is DNA. In one embodiment, the polynucleotide comprises a promoter operably linked to a sequence encoding the gRNA.

In still another aspect, the invention includes a viral vector comprising the polynucleotide.

In a further aspect, the invention provides a viral vector comprising a polynucleotide that encodes an HTRA1 polypeptide, wherein the polynucleotide comprises is a human codon optimized sequence encoding HTRA1 operably linked to a promoter. In one embodiment, the viral vector is a retrovirus, a lentivirus, a herpes virus, or an adeno-associated virus (AAV).

In one embodiment, the promoter of the polynucleotide or the viral vector is an RPE specific promoter.

In another aspect, the invention includes a HTRA1 activating system comprising: (a) a vector comprising a DNA sequence encoding a gRNA; and (b) a vector comprising a DNA sequence encoding a fusion protein comprising a Cas protein domain fused to a transcriptional activator domain, wherein the Cas protein domain lacks nuclease activity. In one embodiment, the vector in (a) and the vector in (b) are different vectors.

In still another aspect, the invention includes a HTRA1 targeting system comprising: (a) a vector comprising a nucleic acid encoding a gRNA disclosed herein; and (b) a vector comprising a nucleic acid encoding a Cas protein. In one aspect, the HTRA1 targeting system further comprises: (c) a vector comprising nucleic acid encoding a template repair sequence, said template repair sequence optionally comprising at least one of SEQ ID NOs: 87-94 or the complement of at least one of SEQ ID NOs: 87-94. In one embodiment, (a) and (b) are the same vector, or (a), (b) and (c) are the same vector.

In still another aspect, the invention includes an isolated cell comprising a gRNA, an RNP, polynucleotide, the viral vector, the activating system, or the targeting system.

In one aspect, invention provides the use of the gRNA, the isolated polynucleotide, the vector, the activating system, the targeting system, or the isolated cell for the preparation of a medicament for treating age-related macular degeneration (AMD). In another aspect, the guide RNA, the isolated polynucleotide, the vector, the activating system, the targeting system, or the isolated cell are used for the preparation of a medicament for treating age-related macular degeneration (AMD). In one embodiment, the subject being treated (a) exhibits a Chr 10 AMD clinical phenotype; (b) has a chromosome 10 risk allele; (c) is homozygous for the chromosome 10 risk allele; or (d) does not have a chromosome 1 risk allele.

The invention further includes, a method for increasing HTRA1 expression in a cell comprising expressing the activating system or the targeting system, in the cell.

In yet another aspect, the invention includes a method of treating, preventing development of, slowing progression of, reversing or ameliorating symptoms and signs of Chr 10 AMD in a subject, comprising administering an agent(s) that increases HTRA1 expression in RPE cells or horizontal cells or photoreceptor cells of the subject. In one embodiment, the subject exhibits a Chr 10 AMD clinical phenotype. In another embodiment, the subject has a chromosome 10 risk allele. In yet another embodiment, the subject is homozygous for the chromosome 10 risk allele. In still another embodiment, the subject does not have a chromosome 1 risk allele. In one aspect, the transcription of an endogenous HTRA1 gene sequence is increased. In one embodiment, the agent is a ribonucleoprotein complex comprising: (a) a fusion protein of an enzymatically inactive Cas protein domain and a transcriptional activator domain and (b) a guide RNA. In one embodiment of this aspect, the enzymatically inactive Cas protein is dCas9. In one embodiment, the ribonucleoprotein complex binds in the HTRA1 promoter region. In another embodiment, the ribonucleoprotein complex binds in the HTRA1 enhancer region. In one embodiment, the transcriptional activator domain binds an LHX2 binding motif. In yet another embodiment, the agent is a ribonucleic acid complex comprising a guide RNA and a Cas protein. In another aspect, the subject carries a risk allele in the HTRA1 gene enhancer, and the agent is a combination comprising (a) ribonucleic acid complex comprising a guide RNA and a Cas protein and (ii) a template repair polynucleotide comprising sequence of a non-risk allele corresponding to the risk allele.

### BRIEF DESCRIPTION OF THE DRAWINGS

FIG. 1 shows in situ hybridization of HTRA1 mRNA in human retina.

FIG. 2 shows microarray analysis of HTRA1 mRNA expression in the Newman data sets.

FIGS. 3A and 3B show qRT-PCR analysis of HTRA1 mRNA expression in the DiaxonHit (A) and Newman (B) data sets (human ocular tissues).

FIGS. 4A and 4B show allele-specific expression of HTRA1 in human ocular tissues (RPE-choroid and neural retina) and RPE scrapes, respectively. The amount of HTRA1 mRNA containing the risk allele at rs1049331 relative to the amount of HTRA1 mRNA with the non-risk allele was determined.

FIGS. 5A and 5B show polarized expression of HTRA1 protein in hTERT-RPE1 and differentiated human fetal RPE cells.

FIG. 6 shows the amounts of HTRA1 protein detected in various tissue extracts from human eye.

FIG. 7. shows the predicted and observed isoforms of long non-coding RNA IncSCTM1 (LOC105378525).

FIG. 8 shows allele-specific expression of human HTRA1 mRNA and IncSCTM1 RNA in RPE-choroid and retina.

FIG. 9 shows the consensus binding motif for LHX2 protein and disruption of the motif by the risk allele at the rs36212733 polymorphic site.

FIGS. 10A and 10B show the binding of LHX2 to Chr10 probes containing the non-risk (WT) sequence at rs36212733 but weaker binding to the risk sequence and no binding to a scrambled sequence.

FIG. 11 shows the result of preferential binding affinity of LHX2 for the non-risk Chr10 probe sequence relative to the risk sequence.

FIGS. 12A and 12B shows HTRA1 protein and mRNA levels in the human retina by Chr10 genotype group

FIGS. 13A and 13B shows HTRA1 protein and mRNA levels in RPE-choroid by Chr10 genotype group.

FIG. 14 shows the sgRNA sequences used to target the HTRA1 promoter.

FIG. 15 shows the HTRA1 and IL1B mRNA levels (quantitative RT-PCR) using total RNA isolated from h1RPE7 cells treated with CRISPR-based SAM components for 72 hours.

FIG. 16 shows HTRA1 mRNA levels (quantitative RT-PCR) in total RNA of h1RPE7 cells mock transfected or transfected with LentiMPH plasmid (MPH), LentiSAM plasmid (SAM) or both (MPH+SAM) for 72 hours.

FIGS. 17A and 17B show levels of HTRA1 (FIG. 17A) and IL1B (FIG. 17B). mRNAs (quantitative RT-PCR) in total RNA of h1RPE7 cells transfected with HTRA1-targeted P7 sgRNA-LentiSAM (P7) or P18 sgRNA (P18) or IL1B-targeted P2 sgRNA-LentiSAM with and without LentiMPH plasmid (MPH) for 72 hours.

FIGS. 18A and 18B show levels of HTRA1 mRNA (quantitative RT-PCR) in total RNA of h1RPE7 cells transfected with different amounts of P7 sgRNA-LentiSAM plasmid (SAM) for 72 hours.

FIGS. 19A and 19B show the results of HTRA1 ELISA, (A) Standard curve of HTRA1 protein; (B) HTRA1 protein levels in cell culture supernatant of h1RPE7 transfected with different amounts (2.5, 5.0 and 7.5 μg) of Ctrl sgRNA- or P18 sgRNA-LentiSAM for 3, 4 and 5 days.

FIGS. 20A and 20B show the results of normalized HTRA1 protein levels (A) and HTRA1 mRNA levels (B) in h1RPE7 cells transfected with different amounts (2.5, 5.0 and 7.5 μg) of Ctrl sgRNA-LentiSAM or P18 sgRNA for 3, 4 and 5 days.

FIG. 21 shows HTRA1 protein levels in cell culture supernatants of h1RPE7 transduced with Ctrl- or P18-LentiSAM viral particles at MOI=20 for 3, 6 and 9 days. Data are plotted as fold-increase relative to Ctrl-LentiSAM.

FIG. 22 shows ENPP-2 protein levels (ng/ml) in cell culture supernatants of h1RPE7 transduced with Ctrl- or P18-LentiSAM lentiviral particles at MOI=20 for 3, 6 and 9 days.

FIGS. 23A and 23B show the results of normalized HTRA1 protein levels (A) and HTRA1 mRNA levels (B) in h1RPE7 cells transduced with MOI 20 of Ctrl sgRNA- or P18 sgRNA-LentiSAM for 3, 6 and 9 days.

FIG. 24 shows HTRA1 mRNA levels in HTRA1 knockdown RPE1 cells transfected with promoterless (None), BEST1, RPE65 or CMV-HTRA1 plasmids using lipofectamine 3000 for 72 hours relative to the control pCTM259 vector.

FIG. 25 shows HTRA1 mRNA levels in RPE1 cells transfected with promoterless (None), BEST1, RPE65 or CMV-HTRA1 plasmids using lipofectamine 3000 for 72 hours relative to the control pCTM259 vector.

FIG. 26 shows a time course kinetics of HTRA1 mRNA expression in RPE1 (HTRA1 KD) cells transfected with AAV2-HTRA1 plasmids driven by the BEST1-, RPE65- or CMV-derived promoters

FIG. 27A-27C show a time course kinetics of HtrA1 protein expression in RPE1 (HTRA1 KD) cells transfected with AAV2-HTRA1 plasmids driven by the BEST1-, RPE65- or CMV-derived promoters.

FIG. 28 shows an example of allele-specific deletion of a region within the Chr10 risk locus encompassing the ARMS2 gene.

FIG. 29 shows the ‘causal’ (regulatory) region and IncSCTM1 on AMD Chromosome 10 Locus. The Chr10 regulatory region is approximately 2-4 kb. A novel IncRNA (designated IncSCTM1) was identified that overlaps this regulatory region. The novel IncRNA contains ARMS2 rs10490924. The arrows show that IncSCTM1 is transcribed in the anti-sense orientation from the HTRA1 promoter and may share a divergent promoter with HTRA1.

FIG. 30 shows microarray analysis of HTRA1 mRNA expression with exon targeted probes in human extramacular RPE-choroid (FIG. 30A), extramacular retina (FIG. 30B), macula RPE-Choroid (FIG. 30C) and macula retina (FIG. 30D) comparing Chr10 non-risk (GG) donors to heterozygous (GT) and homozygous risk (TT) donors.

### DETAILED DESCRIPTION OF THE INVENTION

### 1. Introduction

The inventors have discovered that increasing HTRA1 mRNA and/or protein levels in the eye (e.g., retinal pigment epithelium) provides benefit to subjects with, or at risk of developing, age-related macular degeneration (AMD). In particular, increasing HTRA1 mRNA and/or protein levels provide benefit to patients having manifestations of chromosome10-directed AMD (or “Chr 10 AMD”) or genetically predisposed to developing chromosome-10 directed AMD.

The disclosure provides methods and reagents for treating, preventing development of, slowing progression of, reversing, or ameliorating the symptoms and signs of age-related macular degeneration (AMD) by increasing HTRA1 expression or levels in the eyes of a subject in need of treatment.

Exemplary methods of treating, preventing the development, slowing the progression of, reversing or ameliorating symptoms and signs of Chr 10 AMD are described. The methods comprise administering an agent to a subject, wherein the agent increases HTRA1 mRNA and/or protein levels in RPE cells of the subject, or alternatively horizontal cells or photoreceptor cell. Exemplary methods for increasing HTRA1 mRNA and/or protein levels in cells are described below. In one approach, HTRA1 expression is increased in retinal pigmented epithelial (RPE) cells of the subject. Approaches for increasing the amount of HTRA1 mRNA and/or protein levels in the eye include transcriptional regulation of HTRA1, including upregulation of expression of endogenous HTRA1 using CRISPRa, CRISPR-mediated repair in risk region, gene therapy to introduce HTRA1 protein coding sequences into a cell, reducing degradation of HTRA1 within the cell, and cell therapy. However, the invention is not limited to specific methods and any therapeutically effective approach may be used. As used herein, the “increasing expression of HTRA1” refers to increasing the level of HTRA1 protein by increasing transcription from an endogenous or exogenous gene and the production of a gene product (mRNA or protein). Thus, depending on context, HTRA1 “expression” may refer to production of HTRA1 mRNA or HTRA1 protein. “Increasing expression” may also refer to methods of increasing the amount of HTRA1 protein through mechanisms other than transcription, such as increasing the stability of HTRA1 protein or mRNA in a cell, increasing the rate of translation of HTRA1 mRNA, mRNA recruitment, and direct introduction of exogenous HTRA1 protein into a cell. It will be recognized by the reader that “increasing HTRA1 expression” may refer to increasing the amount of a desired species of HTRA1 protein. For example, in a cell expressing an endogenous HTRA1 protein associated with a risk genotype, “increasing HTRA1 expression” may refer to increasing the amount of HTRA1 protein with a sequence not associated with the risk genotype. In one approach, HTRA1 serine protease activity is increased. The term “upregulate” may be used to refer to an increase in the transcription (e.g. amount of mRNA production) by at least 10%, or at least 20%, or at least 30%, or at least 50% relative to a control cell not treated with an agent.

The discovery that increasing HTRA1 levels provides benefit to patients was unexpected. The consensus in the AMD field has been that overexpression, not under-expression, of HTRA1 is associated with increased risk of developing AMD and that HTRA1 levels or activity should be reduced or inhibited to treat AMD. For example, Dewan et al (2006) reported the risk allele (A) of an HTRA1 promoter SNP at rs11200638 is associated with enhanced HTRA1 transcription in cultured RPE cells. Yang et al (2006) also reported that circulating lymphocytes from individuals homozygous for the risk allele (AA) express higher levels of HTRA1 mRNA as compared to lymphocytes from individuals homozygous for the non-risk allele (GG). Chan et al. (2007) reported that HTRA1 expression is up-regulated at the mRNA level in archived eyes of patients with AMD and concluded that the enhanced expression of HTRA1 was the cause of active neovascularization in the macular lesions of wet AMD. Likewise, Vierkotten et al. (2011) reported that overexpression of HTRA1 correlates with ultrastructural changes in the elastic layer of Bruch's membrane, and suggested that HTRA1 contributes to the pathophysiology of AMD. Jones et al. (2011) reported that overexpressing human HTRA1 in mouse retinal pigment epithelium was associated with induced cardinal features of Polypoidal Choroidal Vasculopathy (“PCV”), including branching networks of choroidal vessels, polypoidal lesions, severe degeneration of the elastic laminae, and tunica media of choroidal vessels. DeAngelis (US patent publication 2013/0122016) proposed slowing the progression of age-related macular degeneration in a subject by reducing the expression of the HTRA1 gene or reducing the biological activity of the HTRA1 gene product. Wu et al. (US patent publication 2013/0129743) proposed using monoclonal antibodies that bind HTRA1 and inhibit HTRA1 enzymatic activity for treatment of AMD.

### 2. HTRA1 Regulatory Elements Associated with AMD

We have identified targets and methods for treating AMD by increasing the levels of HTRA1. Our discoveries are based, in part, on research described in the Examples.

Using donor eyes from human subjects homozygous or heterozygous for the AMD risk allele at rs10490924 (GT, TT) and from control subjects homozygous for non-risk (GG), we observed that donors having AMD risk alleles had lower HTRA1 mRNA expression, as compared to controls. Importantly, the data demonstrated that the reduction of HTRA1 mRNA levels in risk patients is tissue specific: The reduction was detected in RPE, but not in neural retina or choroid. Further, a comparison of HTRA1 protein and mRNA levels in human extramacular retina and RPE-choroid as function of age in donors with or without risk at the Chr10 locus demonstrated that in the retina HtrA1 levels are relatively unchanged with age, independent of a subject's Chr10 risk status, but in the RPE-choroid there is a significant increase with age in HtrA1 mRNA and protein levels of donors without Chr10 risk, as compared to donors with risk alleles.

4 kb AMD Risk Region

An HTRA1 allele-specific expression assay was used to narrow the region of chromosome 10 that is causal for AMD. The assay used mRNA derived from human donors eyes with rare recombination events within the AMD-associated ARMS2/HTRA1 LD block. We mapped the region associated with decreased HTRA1 mRNA to an approximately 4 kb regulatory region (between and including rs11200632 and rs3750846) upstream from the HTRA1 coding sequence. The 4 kb region includes rs10490924 (ARMS2 A69S). The same genetic region was found to be associated with elevated risk of AMD disease by Grassmann et al. (Genetics 2017) in their analysis of recombinant haplotypes in case/control studies. The finding that the 4 kb region we identified as associated with allele-specific expression of HTRA1 matches a region associated with AMD risk strongly implies that risk-associated reduction in HTRA1 expression leads to an increase in AMD incidence. This region is designated as the “4 kb AMD risk region,” or, equivalently the “4 kb risk region,” “4 kb regulatory region,” ‘4 kb causal region” or “4 kb region.”

2 kb AMD Risk Region

Using data describing epigenetic markers of transcriptional activation, we identified an approximately 2 kb genomic region (corresponding to Chr10:122454508-122456564 (hg38)) that overlaps the 4 kb region and regulates HTRA1 transcription. In RPE cells this region is characterized by markers of transcription activation, including H3K27 acetylation, but this region does not show H3K27 acetylation in retina tissue or in data from ENCODE using various non-RPE cell lines, suggesting that the chromatin in this region is active in RPE tissue but not in other cell types. The “2 kb AMD risk region,” (SEQ ID NO: 14) is equivalently referred to as the “2 kb risk region,” “2 kb regulatory region,” “2 kb causal region” or “2 kb region.” The 2 kb AMD risk region is also referred to as the “HTRA1 enhancer region.”

HTRA1 Promoter

The HTRA1 “promoter sequence” comprises CRISPRa (CRISPR activation) target sequences and other features. The promoter region sequence is provided in the following sequences. Unless indicated otherwise or clear from context, references to “promoter” herein is intended to refer to each of these ranges of sequence. SEQ. ID NO: 8 (300 bp) shows the HTRA1 primary promoter sequence including 300 bp 5′ from the putative transcription start site. SEQ. ID NO: 5 (469 bp) shows the extended promoter sequence with additional upstream sequence. SEQ. ID NO: 7 (400 bp) shows the primary promoter sequence plus 100 bp of 5′ UTR sequence. The “native promoter region” is SEQ ID NO:13 (853 bp) with both promoter and UTR sequence.

### 3. Increasing HTRA1 Expression by Transcription Activation

In some approaches, gene therapy is used to enhance expression of endogenous HTRA1 in RPE cells. In some approaches, gene therapy involves delivering a transcriptional activator that can bind to a transcriptional regulatory region of HTRA1 and promote the transcription of HTRA1. In one approach a targeting moiety (e.g., a Cas protein-guide RNA complex) that binds the HTRA1 promoter region and positions a transcriptional activator in proximity to the HTRA1 promoter region is used. In a related approach a targeting moiety binds the HTRA1 enhancer region and positions the transcriptional activator in proximity to a transcription activator binding motif.

In some approaches, the transcriptional activator activity is provided by a fusion protein of a transcriptional activator and a DNA targeting protein. A DNA targeting protein disclosed herein can be a DNA targeting protein that binds to the HTRA1 transcriptional regulatory region. Various platforms using DNA targeting proteins may be used, as discussed below. In certain embodiments “CRISPRa” (CRISPR activation) methods are used. In some cases, a transcriptional activator is provided as fusion protein in which a nuclease-deficient type-II CRISPR-associated protein (Cas) is the DNA targeting protein. Generally, the Cas is modified so that it lacks endonuclease activity (i.e., a “nuclease-dead Cas” or “dCas”). In some approaches the Cas is dCas9. In some approaches the Cas is dCas12a. In some approaches the Cas is any Cas protein with modifications that render it nuclease deficient. As noted above and elsewhere herein the invention is not limited to this method of activation and non-Cas proteins or moieties that direct a transcriptional activator to a promoter or enhancer region can be used.

Thus, in some aspects of the invention, dCas9 is used to mediate upregulation of HTRA1 expression in a CRISPR activation (“CRISPRa”) system. In contrast to Cas9, which is widely used in the type II CRISPR/Cas system and generates double stranded breaks in genomic DNA when directed to a target sequence, dCas9 lacks nuclease activity and does not generate double stranded breaks in DNA. Instead, dCas9 may be connected to a transcriptional activator and complexed with a guide RNA (e.g., sgRNA) that specifically hybridizes to a sequence in the HTRA1 promoter region to achieve precise and robust RNA-guided transcription regulation. Use of dCas9-mediated gene activation systems are well known and are described in, e.g., Dominguez et al., 2016, “Beyond editing: repurposing CRISPR-Cas9 for precision genome regulation and interrogation,” Nat. Rev. Mol. Cell. Biol. 17:5-15.

Other nuclease deficient Cas proteins may also be used to recruit a transcriptional activator to a promoter or enhancer site. For example, a dCas12a domain may be fused to a transcriptional activator domain for use in the methods of the invention (see Sarstedt, “Spotlight on Cas12 A search for more type V Cas12 family members turns up unexpected functionality”, Nature Methods, 2019, 16:213-219; Pickar-Oliver et al., “The next generation of CRISPR-Cas technologies and applications”, Nature Reviews Molecular Cell Biology, 2019, 20:490-507; Kleinstiver et al., “Engineered CRISPR-Cas12a Variants with Increased Activities and Improved Targeting Ranges for Gene, Epigenetic And Base Editing”, Nature Biotechnology, 2019, 37:276-282; Xu et al., 2018, “A CRISPR-dCas Toolbox for Genetic Engineering and Synthetic Biology”, J. Mol. Biol. doi.org/10.1016/j.jmb.2018.06.037). In one approach, the Cas 12a is Acidaminococcus sp. BV3L6 (AsCas12a).

Accordingly, in one embodiment, the method disclosed herein comprises administering an agent to the patient, where the administration results in delivery of the dCas-transcriptional activator fusion protein and one or more sgRNAs to the eye (e.g., RPE) of a patient in need of treatment. The fusion protein binds to the HTRA1 regulatory region under the guidance of one or more single guide RNAs (sgRNAs) thereby upregulating HTRA1 transcription. In one approach, the dCas9 is derived from Staphylococcus aureus (“Sa-dCas9”). In one approach, the dCas9 is derived from Staphylococcus pyogenes (“Sp-dCas9”). The dCas9-transcriptional activator fusion protein can take different configurations, such as those disclosed in Dominguez et al., Nat Rev Mol Cell Biol. January; 17(1): 5-15 (2016). In one example, the fusion protein can consist of multiple copies of a transcriptional activator and one copy of dCas9.

3.1 Fusion Proteins for Transcriptional Activation

Transcription factors useful for increasing HTRA1 mRNA or protein expression bind in the HTRA1 promoter or enhancer regions, and the binding results in increased transcription in cells of a subject, e.g., RPE cells, horizontal cells, or photoreceptor cells. The term “transcription factor” encompasses factors that bind in a promoter region, including factors that activate transcription via a transactivation domain (“TAD”) and factors that bind in an enhancer region, including factors that activate transcription via a scaffold, such as a LIM element, that can recruit and assemble additional transcription factors and chromatin remodeling proteins to initiate transcription, or through other mechanisms. See, e.g., Hirai et al., Structure and functions of powerful transactivators: VP16, MyoD and FoxA Int. J. Dev. Biol. 2010; 54(11-12):1589-1596. Members of both classes of transcription factor include a DNA-binding domain, which directs the transcription factor to the target promoter or enhancer element, and an effector domain (e.g., TAD or scaffold or the like).

In one aspect, the invention makes use of fusion proteins that combine the DNA binding and recognition properties of a nuclease deficient Cas/guide RNA system with the transcription effector properties of a transcription factor. Transcription factor effector domains used in the present invention may be from any source but are generally derived from human or viral transcriptional factors or engineered derivatives thereof. Exemplary transcription factors useful for upregulation of HTRA1 transcription include VP16, VP64, VP160 (VP64 consisting of two or more copies of VP16, and VP160 consists of ten tandem copies of VP16); MLL (UniProt ID Q00613), E2A (UniProt ID P15923), HSF1 (UniProt ID Q00613), NF-IL6 (UniProt ID P17676), NFAT1 (UniProt ID Q13469), NFIX (UniProt ID Q14938), NF-kB (UniProt ID Q04206); MEF2A (Potthoff & Olson (2007), “MEF2: a central regulator of diverse developmental programs,” Development 2007; 134(23):4131-4140; UniProt ID Q02078); and YY1 (Weintraub et al. (2017), “YY1 Is a Structural Regulator of Enhancer-Promoter Loops”, Cell 2017; 171:1573-88; UniProt ID P25490), LHX2 (ZIBETTI et al., “Epigenomic profiling of retinal progenitors reveals LHX2 is required for developmental regulation of open chromatin”, Communications Biology, Apr. 25, 2019, Pages 1-13, 2, UniProt ID P50458).

Human transcription factors (TFs) or transcriptional activators are well known and well-characterized. For example, Lambert et al. describe over 1,600 likely human transcription factors (Lambert et al., 2018, “The Human Transcription Factors” Cell 172:650-665). Also see Fulton et al. (2009), “TFCat: the curated catalog of mouse and human transcription factors”, Genome Biol 2009; 10: R29, Vaquerizas et al. (2009), “A census of human transcription factors: function, expression and evolution”, Nat. Rev. Genet. 2009; 10: 252-263, Wingender et al. (2015), “TFClass: a classification of human transcription factors and their rodent orthologs”, Nucleic Acids Res. 2015; 43: D97-D102. Transcription factor binding motifs are known and are described in collections such as TRANSFAC (Matys et al. (2006), “TRANSFAC and its module TRANSCompel: transcriptional gene regulation in eukaryotes”, Nucleic Acids Res. 2006; 34: D108-D110), JASPAR (Mathelier et al. (2016), “JASPAR 2016: a major expansion and update of the open-access database of transcription factor binding profiles”, Nucleic Acids Res. 2016; 44: D110-D115), HT-SELEX (Jolma et al. (2013), “DNA-binding specificities of human transcription factors”, Cell 2013; 152: 327-339; Jolma et al. (2015), “DNA-dependent formation of transcription factor pairs alters their binding specificity”, Nature 2015; 527: 384-388; Yin et al. (2017), “Impact of cytosine methylation on DNA binding specificities of human transcription factors”, Science 2017; 356: eaaj2239), UniPROBE (Hume et al. (2015), “UniPROBE, update 2015: new tools and content for the online database of protein-binding microarray data on protein-DNA interactions”. Nucleic Acids Re. 2015; 43: D117-D122), and CisBP (Weirauch et al. (2014), “Determination and inference of eukaryotic transcription factor sequence specificity”, Cell 2014; 158: 1431-1443).

Fusion proteins in which a transcription factor effector domain is combined with a nuclease deficient DNA binding domain can also be made using programmable nucleases other than Cas proteins, including, for example, with TALENs and ZFNs.

3.2 Guide RNAs

In some approaches, a targeting moiety includes an RNA with a region of complementarity to the DNA target. The RNA can be referred to as a guide RNA (gRNA) and the region of complementarity can be referred to as the guide sequence. The “guide sequence” of a gRNA is the sequence that confers target specificity. It hybridizes with (i.e., it is the reverse complement of) the opposite strand of a target sequence. In nature, many CRISPR systems include two RNA molecules: tracrRNA, which binds the Cas endonuclease, and crRNA, which binds to the DNA target sequence. Some CRISPR systems (e.g., CRISPR Cas12a/Cpf1) require only crRNA. In research and biomedical applications it is more typical to use a chimeric single guide RNA (“sgRNA”), which is a crRNA-tracrRNA fusion that obviates the need for RNase III and crRNA processing in general, and which binds both the Cas and target. It will be understood that, except where apparent from context, reference to a “sgRNA” encompasses any targeting method using any suitable guide RNA with appropriate binding specificity (e.g., a sgRNA, crRNA, or other RNA comprising the guide sequence).

The most commonly used sgRNA's are approximately 100 base pairs in length. The programmable targeting sequence comprises approximately 20 bases at or near the 5′ end of the sgRNA. By programming this sequence the CRISPR Cas9 system or other Cas system can be targeted towards any genomic region complementary to that sequence.

Methods for designing sgRNAs that target a specified genomic region are well known in the art. See Doench et al. Nature Biotechnology 34:184-191, 2016; Horlbeck et al. eLife. 5, e19760 (2016); Doench et al., Nt. Biotechnol 34(2): 184-191 (2016); Cui et al., “Review of CRISPR/Cas9 sgRNA Design Tools. Interdiscip. Sci. 2018, 10:455-465; Jensen, 2018, Design principles for nuclease-deficient CRISPR-based transcriptional regulators” FEMS Yeast Research, 18:4, and PCT Publication No. WO2018107028. Methods and tools for designing such sgRNAs are also commercially available, for example, from Dharmacon, Inc., as published in websites, such as dharmacon. horizondiscovery.com/applications/crispra-transcriptional-activation-for-gene-overexpression or benchling.com/academic; and Broad institute.org/gpp/public/analysis-tools/sgrna-design-help-crisprai.

In one aspect the invention provides a guide RNA (gRNA) comprising guide sequence with at least 10 contiguous nucleotides corresponding to a target sequence in the HTRA1 promoter. In some cases the guide RNA is an sgRNA. In some approaches the guide sequence comprises at least 10, at least 15, at least 20, or at least 25 nucleotides. In some cases the guide sequence is 20 nucleotides in length. In some embodiments the invention provides a guide RNA comprising a guide sequence, where the guide RNA is complexed with a Cas (such as Cas9) lacking nuclease activity (e.g., dCas9). In some cases the invention provides a guide RNA, such as a sgRNA, complexed with a Cas fusion protein including a Cas DNA binding domain and a transcription activator. Typically, the Cas is a nuclease deficient dCas (such as dCas9).

In some cases the DNA target sequence (e.g., in the HTRA1 promoter or enhancer) is contiguous with a protospacer adjacent motif (PAM) recognized by the Cas protein. For example, Cas9 generally requires the PAM motif NGG for activity. Thus, in some systems, certain target sequences (and accordingly certain guide sequences) will be preferred based on the proximity of the target sequence to a PAM. However, some Cas proteins, including variants of Cas9, have flexible PAM requirements (see Karvekis et al., 2019, “PAM recognition by miniature CRISPR-Cas14 triggers programmable double-stranded DNA cleavage.” bioRxiv. https://doi.org/10.1101/654897; Legut et al., 2020, “High-Throughput Screens of PAM-Flexible Cas9”, Cell Reports 30:2859-2868; Jakimo et al., 2018 Cas9 with Complete PAM Recognition for Adenine Dinucleotides bioRxiv doi.org/10.1101/429654; Esvelt K M, Mali P, et al. (2013) Orthogonal Cas9 proteins for RNA-guided gene regulation and editing. Nature Methods, 10(11):1116-1121; Gleditzsch et al., 2019, PAM identification by CRISPR-Cas effector complexes: diversified mechanisms and structures. RNA Biol. 2019 April; 16(4): 504-517; Tang et al., 2019, Efficient cleavage resolves PAM preferences of CRISPR-Cas in human cells Cell Regeneration 8:44-50) and other Cas proteins are PAM-independent (e.g., Cas14a1). Exemplary PAMs include SpCas9 from Streptococcus pyogenes NGG; SpCas9 from Streptococcus pyogenes NRG; StCas9 from Streptococcus thermophilus NNAGAAW; NmCas9 from Neisseria meningitidis NNNNGATT; SaCas9 from Staphylococcus aureus NNGRRT; SaCas9 variant (KKH SaCas9) NNNRRT; SpCas9 D1135E variant NGG; SpCas9 VRER variant NGCG; SpCas9 EQR variant NGAG; SpCas9 VQR variant NGAN or NGNG 3′; AsCpf1 from Acidaminococcus, LbCpf1 from Lachnospiraceae TTTN; FnCpf1 from Francisella novicida strain U112 TTN and/or CTA; C2c1 from four major taxa: Bacilli, Verrucomicrobia, a proteobacteria, and d-proteobacteria TTN (N=A, T, C or G; R=A or G; W=A or T) described in Zhao et al., 2017, CRISPR-offinder: a CRISPR guide RNA design and off-target searching tool for user-defined protospacer adjacent motif. Int J Biol Sci 2017; 13(12):1470-1478. Thus, although the commonly used Cas9 protein requires a target sequence and a contiguous NGG PAM, other naturally occurring or engineered Cas proteins have relaxed or no PAM requirement. As a result, through judicious choice of CRISPR/Cas systems, the practitioner will be able to select almost any sub-sequence in an HTRA1 transcriptional regulatory region as the target for recruiting a transcriptional activator. In one approach an HTRA1 promoter target is found in SEQ ID NO:13. In some approaches the HTRA1 promoter target is found in SEQ ID NO:5. In some approaches the HTRA1 promoter target is found in SEQ ID NO:8. In some approaches the HTRA1 promoter target is found in SEQ ID NO:13. In some approaches the HTRA1 promoter target is found in SEQ ID NO:7. In one approach an HTRA1 enhancer target is found in SEQ ID NO:14 (2 kb region). In some approaches the HTRA1 target is found in SEQ ID NO: 14 (2 kb region) or SEQ ID NO:34 (4 kb region).

According to the present invention a guide sequence hybridizes to a target sequence that includes at least 10, at least 15, at least 20, or about 20 contiguous nucleotides of an HTRA1 promoter sequence (SEQ ID NO:5, SEQ ID NO: 7, SEQ ID NO: 8, or SEQ ID NO: 13) or of the reverse complement of SEQ ID NO:5, SEQ ID NO: 7, SEQ ID NO: 8, or SEQ ID NO: 13 or an HTRA1 enhancer sequence (SEQ ID NO:14 or 34) or of the reverse complement of SEQ ID NO: 14 or 34.

In some cases the guide sequence comprises 10 or more contiguous bases of a promoter sequence given above. Examples of sequences that comprise 10 contiguous bases of SEQ NO: 13 include GTCCCAACGG; TCCCAACGGA; CCCAACGGAT; CCAACGGATG; CAACGGATGC, etc., or the reverse complement thereof. Sequences that comprise 10 or more contiguous bases of SEQ NO: 13 include a sequence encoding bases 1 to 10 of SEQ ID NO: 13, encoding bases 2-11 of SEQ ID NO:13, encoding bases 3-12 of SEQ NO: 13, etc. Sequences that comprise 10 or more contiguous bases of SEQ NO: 13 include a sequence in which a nucleotide is base X of SEQ ID NO: 13, where X is 1 to 300, and the 10 contiguous bases extend to X+Y of SEQ ID NO: 13 where Y is 9, 10, 11, 12, 13, 14, 15, 16, 17, 18, 19, 20, 21, 22, 23, 24, 25 or more with the proviso that the guide sequence is often less than 50 bases in length. A sequence of 15 contiguous nucleotides, or at least 20 can be described in the same way. For example, in some cases the guide sequence comprises 15 or more contiguous bases of SEQ NO: 13 (GTCCCAACGGATGCA; TCCCAACGGATGCAC; CCCAACGGATGCACC; CCAACGGATGCACCA; CAACGGATGCACCAA) or the reverse complement thereof.

It will be recognized that each of the 10, 15 or 20 nucleotide sequences, and complements thereof can be listed in tables and individual sequences, or combinations of sequences, could be independently selected from such tables for inclusion into a set, or for exclusion from a set. That is, such tables would describe and provide basis for selecting or excluding individual sequence and combinations. It will be understood that the description above is intended to that the place of, and have the same content of, such tables.

As described in Example 9 (§ 14.9), we designed sgRNAs with guide sequences that correspond to target regions in SEQ ID NO:13, denoted SEQ ID NO: 15-33, and tested their ability to activate transcription from the HTRA1 promoter. In some approaches, the gRNA used in the present methods target a sequence comprising one SEQ ID NO: 15-33.

In some embodiments, the target sequence in the gene of interest may be complementary to the guide region of the sgRNA. In general, there is exact complementarity or identity between a guide sequence of a gRNA and its corresponding target sequence may be less than 100% In some embodiments, the degree of complementarity or identity between a guide region of a sgRNA and its corresponding target sequence may be less than 100% although 100% identity is desired to avoid off-target effects. In some embodiments, the guide region of a sgRNA and the target region of a gene of interest may be at least 95% identical (e.g., one mismatch out of 20), at least 90% identical or at least 85% identical.

3.3 Delivery

Methods of delivery to cells of a Cas protein (e.g., Cas9 and dCas9) fused to a transcriptional activator protein and gRNA are known. The fusion protein can be delivered in protein form (e.g., via microinjection). More often, the fusion protein is delivered in DNA form, e.g., in a suitable vector that can be introduced into RPE or choroid cells. Generally, DNA encoding the gRNA is cloned into a vector downstream of a promoter (e.g., U6 promoter) for expression. In some approaches, the delivery is by a virus e.g., lentivirus, adeno-associated virus (AAV), as described in Byrne et al., Methods Enzymol. 546, 119-38 (2014); Cong et al., Science (80). 339, 819-823; Hirsch et al., Mol. Ther. 18, 6-8 (2010). In some approaches, the delivery is by cell-derived nanovesicles or other methods. Also see § 7 below.

3.4 Synergistic Activation Mediator

In some approaches, a CRISPRa system known as a synergistic activation mediator (SAM) is used to increase the expression of HTRA1 in Chr 10 AMD patients. The SAM system uses multiple transcription factors, which can further improve the potency of Cas9-mediated gene activation. See, Konermann et al., Nature, January 29; 517(7536) 2015), the relevant disclosure is herein incorporated by reference. In some cases, this system uses two plasmids, one encoding both the sgRNA and a dCas9-transcriptional activator, e.g., dCas9-VP64 molecule, and the other encoding a MS2-TAD fusion protein. The MS2-TAD comprises an MS2 polypeptide and at least one transcription activation domain. In some approaches, the MS2-TAD fusion protein comprises an MS2 polypeptide (SEQ ID NO:56) and the transactivation domains of p65 and HSF1 (MS2-p65-HSF1). The sgRNAs used in the SAM system comprise one or more MS2 binding sequences (e.g., SEQ ID NO.: 55), which can bind to the MS2-TAD fusion protein. In some embodiments, two MS2 binding sequences are included in the gRNA: one in the tetraloop and one on the stem loop of the gRNA. Plasmids encoding MS2-TAD (e.g., MS2-p65-HSF1) are commercially available, e.g., the LentiMPH plasmid (Addgene, Watertown, Mass., USA). Plasmids that encode the sgRNAs and the dCas9-VP64 fusion proteins can be constructed by cloning the suitable sgRNA coding sequences into a plasmid that carries the dCas9-VP64 coding sequence. Plasmids that carry the dCas9-VP64 coding sequence are also commercially available, e.g., the LentiSAM plasmid from Addgene (Watertown, Mass., USA). The sgRNA coding sequences can be clone cloned into the LentiSAM plasmid.

3.5 Enhancer Binding Sites

In some approaches transcription from an endogenous HTRA1 gene is increased by binding a transcription activator to a site in the HTRA1 2 kb (enhancer) region. As discussed in detail below, the inventors have identified a binding motif for the transcription activator LHX2 which overlaps the risk allele of rs36212733. Several lines of genetic and experimental evidence suggest that LHX2 binding to the risk allele is absent or is diminished relative to binding to the non-risk allele, and that this results in reduced expression of HTRA1. In one approach, an LHX2 LIM domain fused to a Cas-protein is combined with an appropriate guide RNA and recruited to the LHX2 risk binding site in a patient carrying the risk allele. The delivery of LHX2 to the vicinity of the risk allele binding site is believed to result in increased gene expression because the tethering of the transcription activator effectively increases the local concentration of LHX2, eliminating or mitigating the negative effect of the risk variation on binding, and resulting in increased expression of HTRA1. In another approach the LHX2 transcription activator is modified to efficiently bind the risk allele sequence, thereby resulting in increased transcription.

Based, in part, on the discovery that a risk allele coincident with a transcription activator binding site may reduce or otherwise affect binding of a transcription activator and result in lower HTRA1 expression, and that recruiting a corresponding transcription activator to the site can increase HTRA1 expression, a review of the 2 kb region was conducted to identify other risk associated sequence variations that are coincident with a transcription activator binding motif. Analysis of the risk alleles (i.e., risk-associated variante) at rs144224550, rs10490924, rs3750848 and rs6212733 identified several binding motifs (in addition to the LXH2 motif) coincident with the risk-associated SNP.

rs144224550 (10:122455084-122455085); Nkx2-5(var2); NFIX;

rs36212733 (10:122455695); MEF2A; HOXB4; ALX3; HOXD3; LBX1; HOXD4; VAX1; LBX1; VSX1; VSX2, LHX9; MNX1; PDX1; EMX1; PRRX2; BARX2;

rs10490924 (10:122454932) RHOXF; Myog; TCF12; ASCL1; Ascl2; NHLH1; BHLHA15 var2

rs3750848 (10:122455799); YY1; BARX2;

According to aspects of the present invention, effector domains from a transcriptional activator listed above, such as LIM domains from LXH2, are positioned (e.g., using CRISPRa) to the corresponding binding motif to increase HTRA1 expression. Notably, NFIX, MEF2A and YY1 are expressed in the RPE at FPKM greater than 1, based on RNA-Seq data. In various aspects of the invention, HTRA1 expression is increased by binding of any one of NFIX, MEF2A and YY1 to its corresponding binding motif.

Generally the guide sequence for an appropriate guide RNA will bind a target sequence with a terminus within 20 bases of the risk associated nucleotide, sometimes within 50 bases, and sometimes within 100 bases.

3.6 Other Programmable Endonucleases

The methods of the invention are generally described within the context of CRISPR/Cas systems. Other systems for (a) sequence-specific (“programmable”) modification of nucleic acids and (b) sequence-specific recruitment of transcription activators to promoter and enhancer regions may also be used. Examples of such systems include a zinc-finger nuclease systems, transcription activator-like effector nuclease (TALEN) systems, Homing endonuclease systems, meganuclease systems or a Cre recombinase systems (e.g., Cre-induced recombination between cryptic loxP sites). Use of zinc-finger nucleases, TALENs, meganucleases and DNA-guided polypeptides such as Natronobacterium gregoryi Argonaute (NgAgo) to modify sequence or expression in cell in the eye are described in Yanik et al., 2017, In vivo genome editing as a potential treatment strategy for inherited retinal dystrophies Progress in Retinal and Eye Research 56:1-18. Also see, Lloyd et al., Frontiers in Immunology, 4(221), 1-7 (2013); Urnov et al., 2010, “Genome editing with engineered zinc finger nucleases,” Nat Rev Genet. 11(9):636-46; Sun et al., 2013, “Transcription activator-like effector nucleases (TALENs): a highly efficient and versatile tool for genome editing,” Biotechnol Bioeng. 110(7):1811-21; Sengupta et al., 2017, “Viral Cre-LoxP tools aid genome modification in mammalian cells” J. Biological Engineering 11:45, (describing lentivirus and adeno associated virus delivery systems for Cre-Lox) and Nagy 2000, “Cre recombinase: the universal reagent for genome tailoring,” Genesis, 26(2):99-109, each incorporated by reference herein. In each genome modification method, a specific nucleic acid sequence is targeted, and a subsequent modification is made. These modifications include the target sequence being edited by homologous recombination, non-homologous end joining, homology-directed repair, histone modification, transcriptional activation, RNA editing, transcriptional repression. In addition, examples of engineering without cleavage are described by Thakore et al. 2018 in “RNA-guided transcriptional silencing in vivo with S. aureus CRISPR-Cas9 repressors” Nat Commun. 9(1):1674 and by Amabile et al. 2016 in “Inheritable Silencing of Endogenous Genes by Hit-and-Run Targeted Epigenetic Editing” Cell 167(1): 219-232.e14. [0060] In some embodiments, the DNA-targeting molecule comprises one or more zinc-finger proteins (ZFPs) or domains thereof that bind to DNA in a sequence-specific manner and that are fused to a nuclease. A ZFP or domain thereof is a protein or domain within a larger protein that binds DNA in a sequence-specific manner through one or more zinc fingers, regions of amino acid sequence within the binding domain whose structure is stabilized through coordination of a zinc ion. The term zinc finger DNA binding protein is often abbreviated as zinc finger protein or ZFP.

Among the ZFPs are artificial ZFP domains targeting specific DNA sequences, typically 9-18 nucleotides long, generated by assembly of individual fingers. ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (−1, 2, 3 and 6) on a zinc finger recognition helix. Thus, in some embodiments, the ZFP or ZFP-containing molecule is non-naturally occurring, e.g., is engineered to bind to a target site of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties.

In some embodiments, the DNA-targeting molecule is or comprises a zinc-finger DNA binding domain, TALEN, or other DNA-targeting protein fused to a DNA cleavage domain to form a targeted nuclease. In some embodiments, fusion proteins comprise the cleavage domain (or cleavage half-domain) from at least one Type IIS restriction enzyme and one or more DNA-targeting protein. In some embodiments, the cleavage domain is from the Type IIS restriction endonuclease Fok I. Fok I generally catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994) Proc. Natl. Acad. Sci. USA 91:883-887; Kim et al. (1994) J. Biol. Chem. 269:31,978-31,982.

The term “programable nuclease” can refer to CRISPR family Cas nuclease or derivative thereof, a transcription activator-like effector nuclease (TALEN) or derivative thereof, a zinc-finger nuclease (ZFN) or derivative thereof, and a homing endonuclease (HE) or derivative thereof.

### 4. Gene-Editing to Replace Risk Forms with Non-Risk Forms

In some approaches, the method of treating, preventing development, slowing progression of, reversing or ameliorating symptoms and signs of Chr 10 AMD, comprises administering an agent to modify the genomic DNA of the patient's RPE cells by converting or substituting one or more of the risk alleles with corresponding non-risk alleles. In certain embodiments the alleles are in the 2.0 kb risk region (see, e.g., Example 5 and FIG. 13).

4.1 Repair of Risk Alleles

In some approaches, gRNAs that target one or more of the risk alleles in the 2 kb region are used. These risk alleles include but not limited to, rs10490924 (the risk allele is T), rs144224550 (risk allele is GT insertion), rs36212731 (the risk allele is T), rs36212733 (the risk allele is C), rs3750848 (the risk allele is G), rs3750847 (the risk allele is T), and rs3750846 (the risk allele is C). Exemplary sgRNAs/guide sequences that can be used to target these risk alleles are shown in Table 1, below. The nucleotides in bold are found in the risk alleles.

TABLE 1
sgRNAs Targeting Risk Alleles

| Name | SEQ | Target SNP | sgRNA Sequence | PAM |
|---|---|---|---|---|
| sgRNA-20 | 36 | rs10490924 | CTGGTAAGCAGAGCTCAGTG | TGG |
| sgRNA-21 | 37 | rs10490924 | AAGGAGCCAGTGACAAGCAG | AGG |
| sgRNA-22 | 38 | rs144224550 | TGGTTAAAATGCAAGCTGCA | GGG |
| sgRNA-23 | 39 | rs144224550 | TTGAATCAGAAATTCTGGAG | TGG |
| sgRNA-24 | 40 | rs36212731 | TGAGCAGGAAAAATGAGCAC | TGG |
| sgRNA-25 | 41 | rs36212731 | CCGTGAGAATATTGAAAGAA | TGG |
| sgRNA-26 | 42 | rs36212733 | TTGTCTAGTTATATATACTA | CGG |
| sgRNA-27 | 43 | rs36212733 | AGACAAATGAGAGAACACAA | AGG |
| sgRNA-28 | 44 | rs3750848 | AATGGTTTACCATTGAATCA | AGG |
| sgRNA-29 | 45 | rs3750848 | GCTTGTCAATTTCTGTCACT | CGG |
| sgRNA-30 | 46 | rs3750847 | AGCCCCAGGCAGCCACCAAA | AGG |
| sgRNA-31 | 47 | rs3750847 | TACAATTCAAACAGAGCCCC | AGG |
| sgRNA-32 | 48 | rs3750846 | GCCTCATGCAACTGATTTAG | GGG |
| sgRNA-33 | 49 | rs3750846 | TTAATGCAAAGATGGACTGC | TGG |

An sgRNA, Cas protein and a template repair polynucleotide comprising sequence of a non-risk allele can be introduced to RPE cells in one or more viral vectors. The template repair sequence generally requires a certain amount of overlap (homology) on each side of the cut site. For repair of single nucleotides, the “homology arms” of the donor template should be approximately 200-500 nucleotides. For larger repair (e.g. the entire 2 kb region), each homology arm should be approximately 500-800 nucleotides. This results in the risk allele being replaced with the wild type allele, thus restoring HTRA1 expression in the RPE cells in Chr10 AMD patients to normal levels. Exemplary repair sequences are shown in Table 2.

TABLE 2
Exemplary Repair Sequences

| Target SNP | Repair Sequence | SEQ ID NO: |
|---|---|---|
| rs10490924 | gatcccagctGctaaaatcca | 87 |
| rs144224550 | attctggagtGGtgccctgcag | 88 |
| rs36212731 | atattctcacGgctttccagt | 89 |
| rs36212732 | tgtgcttgccAtagtatatat | 90 |
| rs36212733 | gtatatataaTtagacaaatg | 91 |
| rs3750848 | tgattcaatgTtaaaccattt | 92 |
| rs3750847 | caagacctttCggtggctgcc | 93 |
| rs3750846 | ggactgctggTctcatgcaac | 94 |

It will be understood that the guide sequence does not have to directly overlap the target SNP. If the sgRNA is near or adjacent to the SNP, that will suffice to allow homology-directed repair of the defective SNP. In this context, “adjacent to” means the closest nucleotide hybridized to a guide sequence nucleotide is within 25 nucleotides from the SNP or other repair site, preferably withing 20 nucleotides, and sometimes within 15 or 10 nucleotides.

4.2 Substitution of a Large Region in the HTRA1 Transcriptional Regulatory Region

In some approaches, a pair of gRNAs are used in the CRISPR/Cas9 system to remove the entire 2 kb risk region in the RPE cells from Chr10 AMD patients. The pair of sgRNAs are designed to target the two nucleotide positions, the region defined by the two positions encompasses the 2 kb risk region. In some approaches, the sgRNAs are SEQ ID NO: 50 and SEQ ID NO: 52. In some approaches, the sgRNA pair are SEQ ID NO: 51 and SEQ ID NO: 52. Introduction of the pair of sgRNAs and Cas9 to the RPE cells results in the region encompassing the 2 kb region being removed. A plasmid encoding a non-risk sequence corresponding to the deleted region and containing 500-800 nt of additional genomic sequence upstream of the 5′ cut site and downstream of the 3′ cut site is introduced to RPE cells such that the non-risk sequence are inserted into the deleted region by homology-directed repair, resulting in the 2 kb risk region being replaced with non-risk sequence. The sgRNA pairs, Cas9, and the plasmid encoding the non-risk template sequence may be introduced in the same or different viral vectors and may be introduced simultaneously or sequentially to increase the HTRA1 expression in the RPE cells of the Chr10 AMD patients.

TABLE 3
sgRNA Targeting the 2 kb Risk Region

| | SEQ | | | |
|---|---|---|---|---|
| | ID | | | PAM |
| Name | NO | Target SNP | sgRNA Sequence | Sequence |
| A1 | 50 | rs10490924 | GAAGGACACAGGAA | AGG |
| (ARMS2-1) | | | CCACCG | |
| A2 | 51 | rs10490924 | CCCAGGACCGATGG | AGG |
| (ARMS2-2) | | | TAACTG | |
| INDEL1 | 52 | ARMS2- | TAATAACCGGGAGA | AGG |
| | | INDEL | GAAAGG | |

### 5. CRISPR/Cas Systems

In some approaches, CRISPR technology is used to introduce one or more nucleotide substitutions to the genomic DNA of the RPE or choroid cells in an individual carrying the Chr 10 risk alleles, thereby substituting one or more of the risk alleles with the corresponding non-risk alleles. The “CRISPR/Cas” system refers to a widespread class of bacterial systems for defense against foreign nucleic acid. CRISPR/Cas systems include type I, II, and III sub-types. Wild-type type II CRISPR/Cas systems use the RNA-mediated nuclease, for example, Cas9, in complex with guide and activating RNA to recognize and cleave foreign nucleic acid. Non-limiting examples of Cas proteins include Cas1, Cas1B, Cast, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known. For example, the amino acid sequence of S. pyogenes Cas9 protein may be found in the SwissProt database under accession number Q99ZW2. Non-limiting examples of mutations in a Cas9 protein are known in the art (see e.g., WO2015/161276), any of which can be included in a CRISPR/Cas9 system in accord with the provided methods. Cas9 homologs are found in a wide variety of eubacteria, including, but not limited to bacteria of the following taxonomic groups: Actinobacteria, Aquificae, Bacteroidetes-Chlorobi, Chlamydiae-Verrucomicrobia, Chlroflexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, e.g., Chylinksi, et al., RNA Biol. 2013 May 1; 10(5): 726-737; Nat. Rev. Microbiol. 2011 June; 9(6): 467-477; Hou, et al., Proc Natl Acad Sci USA. 2013 Sep. 24; 110(39):15644-9; Sampson et al., Nature. 2013 May 9; 497(7448):254-7; and Jinek, et al., Science. 2012 Aug. 17; 337(6096):816-21. Cas9 and its homologs can be used with sgRNAs to introduce specific modifications to the genomic regions of interest, e.g., the genomic region comprising one or more Chr 10 risk alleles.

Other RNA-mediated nucleases that can also be used in a CRISPR/Cas system to convert the risk alleles in the 2 kb risk region include, for example, Cas 12a and Cascade/Cas3. See Pickar-Oliver and Gersbach, Nature, vol. 20, August 2019, the relevant portion of which is herein incorporated by reference. Cas 12a recognizes a target sequence with complementarity to the spacer in the crRNA (sgRNA) positioned next to a 3′ PAM. Target recognition results in generation of a staggered DNA double-strand break. Cascade/Cas3 is a multimeric complex that targets a DNA that has complementarity to the spacer portion of a crRNA and that is positioned next to a 3′PAM and generates a single strand nick.

Accordingly, in some approaches, the method comprises introducing into the RPE cells of an individual a CRISPR system, where the system may comprise a Cas9 protein and a guide RNA (e.g., an sgRNA) that hybridizes with the target sequence. The sgRNA and Cas9 can be expressed from the same or different vectors of the system. In addition, a donor vector encoding the non-risk nucleotide sequence that overlaps the cut site is co-delivered with the CRISPR-Cas9 plasmids. The guide RNA targets a sequence comprising or near the risk allele, and the Cas9 protein cleaves the genomic DNA molecule. The cut genomic DNA is repaired by homologous recombination using the donor sequence plasmid, resulting in a change in the genomic sequence from the risk allele to the non-risk allele. Any of the risk alleles described above can be converted into non-risk alleles. In some approaches, the entire 2.0 kb region (see Example 5) or the entire 4 kb AMD risk region (see Example 3) is converted such that it comprises no Chr 10 risk alleles, which restores HTRA1 expression in RPE cells.

5.1 Cas9 Nickase

In some approaches, a Cas9 nickase based CRISPR/Cas system may be used to correct the individual risk alleles. Compared to unmodified Cas9 protein, a cas9 nickase protein contains a mutation in one of its nuclease domains. The Cas9 nickase protein thus would “nick” target DNA, i.e., cause a single-strand DNA cut at a target site rather than a double-strand cut as with unmodified Cas9. In one example, dCas9 contains a mutation in each of its two nuclease domains, while a Cas9 nickase contains a mutation in only one of its nuclease domains. As in other CRISPR approaches described herein, the guide RNA is able to target the Cas9 nickase to a specific genomic location. The cas9 nickase protein is introduced with base editing proteins, such as cytidine deaminases (e.g., APOBEC1, AID, APOBEC3G, or CDA1), which convert cytosine bases to uracil bases (which have the base-pairing properties of thymine bases), and adenine deaminases, which convert adenosine bases to inosine bases (which have the base-pairing properties of guanosine). These conversions have the effect of changing a C-G base pair to a T-A base pair (cytidine deaminases) or an A-T base pair to a G-C base pair (adenine deaminases). In some cases, the Cas9 nickase based on CRISPR/Cas system also include an inhibitor of cellular DNA repair response to promote base editing efficiency. For example, uracil DNA glycosylase inhibitor can be used to prevent uracil DNA glycosylase enzyme in cells from catalysing removal of uracil bases from DNA. Removal of uracil can lead to reversion of an edited uracil back to cytosine. Cas9 nickase based gene editing systems are well-known and are described in, e.g., Komor et al., Nature, 533(7603): 420-424 (2016). In one example, the system comprises a cytidine deaminase (APOBEC1) fused to the N-terminus of Cas9 nickase with a 16 amino acid residue XTEN linker, and uracil DNA glycosylase inhibitor (UGI) fused to the C-terminus of Cas9 nickase (e.g., editor BE3 in Komor Et al., Nature, 533(7603): 420-424 (2016)). Such systems are able to efficiently edit C-G base pairs to T-A base pairs in vivo with low rates of indel (i.e., small deletions or insertions) formation and off-target activity (i.e., editing or indel formation at genomic locations other than the locus targeted by the guide RNA). In some cases, an editing protein linked to Cas9 nickase can target multiple bases, depending on the length of the linker sequence. For example, APOBEC1 fused to dCas9 with a 16 residue XTEN linker can deaminate cytosine bases within a window of approximately five nucleotides, typically from positions 4 to 8 within the protospacer sequence, counting the end distal to the protospacer-adjacent motif as position 1 (see, e.g., Komor et al., Nature, 533(7603): 420-424 (2016)). In some approaches, guide RNA sequences can be designed to target one or multiple bases within such a window.

In some approaches, vector sequences expressing the Cas9 nickase and guide RNAs are integrated into host cell DNA as transgenes. For example, gene sequences encoding the APOBEC1-XTEN-Cas9 nickase-UGI fusion described above, along with guide RNAs, may be integrated in a plasmid and transfected into cells in a population. In some approaches, delivery of the CRISPR base editing system components is by a virus, e.g., lentivirus or adeno-associated virus (AAV). In some instances, the DNA sequences encoding the CRISPR base editing system components are too large for packaging in AAV, which has a genome packaging size limit of less than 5 kilobases. In one approach, the CRISPR base editing system components can be delivered using a dual-AAV strategy employing intein sequences. Inteins are segments of a protein that are able to excise themselves and splice together the remaining protein portions. Inteins of the precursor protein can come from two genes, in which case they are called split inteins. In one example of such a dual-AAV strategy, as described in Levy et al., Nat. Biomed. Eng., 4(1): 97-110 (2020), cytosine base editors such as BE3 described above can be divided in halt with each half fused to half of a fast-splicing split intein. Sequences encoding these products can be incorporated into two separate AAV genomes, along with sequences expressing guide RNAs, and co-transduced into cells. When both products are expressed in a cell, they can be spliced together, leading to reconstitution of a full-length base editor such as BE3.

### 6. Increasing HTRA1 Expression Using Gene Therapy by Introducing Exogenous HTRA1-Protein Encoding Polynucleotides

In one embodiment, treating AMD involves gene therapy to enhance HTRA1 expression in RPE cells. Gene therapy is a well-known technology and described in e.g., Moore et al., 2017, “GENE THERAPY FOR AGE-RELATED MACULAR DEGENERATION” Expert Opinion on Biological Therapy 17:10: 1235-1244; Aponte-Ubillus et al., 2018, “MOLECULAR DESIGN FOR RECOMBINANT ADENO-ASSOCIATED VIRUS (RAAV) VECTOR PRODUCTION” Applied microbiology and biotechnology 102.3:1045-1054; Ochakovski et al., 2017, “RETINAL GENE THERAPY: SURGICAL VECTOR DELIVERY IN THE TRANSLATION TO CLINICAL TRIALS” Frontiers in Neuroscience 11; Schön et al., 2015, “RETINAL GENE DELIVERY BY ADENO-ASSOCIATED VIRUS (AAV) VECTORS: STRATEGIES AND APPLICATIONS” European Journal of Pharmaceutics and Biopharmaceutics 95:343-352; Naso et al., 2017, “ADENO-ASSOCIATED VIRUS (AAV) AS A VECTOR FOR GENE THERAPY” BioDrugs 31:317; Dunbar et al., 2018, “GENE THERAPY COMES OF AGE” Science 359:6372; Penaud-Budloo et al., 2018., “PHARMACOLOGY OF RECOMBINANT ADENO-ASSOCIATED VIRUS PRODUCTION” Molecular Therapy: Methods & Clinical Development 8:166-180; each of which is incorporated by reference for all purposes.

6.1 Expression of an Exogenous HTRA1 Protein

In some approaches, gene therapy is performed to introduce an exogenous HTRA1 protein in RPE cells. In some approaches, the introduced exogenous HTRA1 has the same amino acid sequence as the native HTRA1 protein (SEQ ID NO: 2). In some approaches, the exogenous HTRA1 protein has an amino acid sequence that differs from SEQ ID NO: 2 but shares at least 90%, at least 91%, at least 92%, at least 93%, at least 94%, at least 95%, at least 96%, at least 97%, at least 98%, or at least 99% amino acid sequence identity with the amino acid sequence of SEQ ID NO: 2. In some approaches, the exogenous HTRA1 protein differs from SEQ ID NO:2 (i.e., HTRA1 variants) but maintains the serine protease activity of HTRA1. Serine protease activity of HTRA1 can be measured using methods well known in the art, for example, as described in Grau et al., Proc. Natl. Acad. Sci. U.S.A. April 26, 102 (17) 6021-6026 (2005). For the purpose of this disclosure, maintaining the serine protease activity refers to that an exogenous HTRA1 protein with at least 50%, at least 60%, at least 70%, at least 80%, at least 90%, or at least 95% serine protease activity of the native HTRA1 protein (SEQ ID NO: 2).

In some approaches, the gene therapy incudes administering a vector comprising a nucleic acid sequence (“cargo”) that encodes an exogenous HTRA1 protein of SEQ ID NO:2 or variants as described above such as variants with serine protease activity of HTRA1. In some approaches, the nucleic acid sequence includes SEQ ID NO: 1. In some approaches, the nucleic acid sequence shares a significant sequence identity, e.g., at least 90%, at least 91%, at least 92%, at least 93%, at least 94%, at least 95%, at least 96%, at least 97%, at least 98%, or at least 99% nucleic acid sequence identity with SEQ ID NO: 1. In some approaches, the nucleic acid sequence encoding the exogenous HTRA1 protein has significant sequence identity with SEQ ID NO:2, e.g., at least 90%, at least 91%, at least 92%, at least 93%, at least 94%, at least 95%, at least 96%, at least 97%, at least 98%, or at least 99%, with SEQ ID NO: 2. In some cases, nucleic acids encoding the HTRA1 proteins include sequences that comprise deletions, insertions, or substitutions relative to the native HTRA1 nucleic acid sequence, which still result in a polynucleotide encoding a polypeptide with serine protease activity.

HTRA1 nucleic acids and polypeptides can also be modified through chemical or enzymatic alterations using methods well known to those of skill in the art. For example, sequences can be modified by the addition of lipids, sugars, peptides, organic or inorganic compounds, by the inclusion of modified nucleotides or amino acids, or the like. Accordingly, the HTRA1 nucleic acids and proteins can also be conjugated to another moiety (e.g., a reporter protein), the presence of which may facilitate the detection or serve other purposes.

6.2 Vectors for Gene Therapy

Viral vectors suitable for introducing an HTRA1-encoding sequence include, for illustration and not limitation, adenovirus, AAV2 virus, lentivirus, bovine papilloma virus (BPV-I), or Epstein-Barr virus (pHEBo, pREP-derived and p205). Non-viral systems, such as naked DNA formulated as a microparticle, may be used. See § 7, below.

6.3 Promoters for Gene Therapy Vectors

In some approaches, the transgene (e.g., the exogenous HTRA1 gene, or the recombinant nucleic acid the expression of which in the RPE cells can increase the expression of the endogenous HTRA1 gene) that is delivered comprises a protein coding sequence operably linked to a promoter sequence. In some approaches, the promoter is heterogeneous to the HTRA1 polynucleotide. In some approaches, the promoter is a native HTRA1 promoter (e.g., SEQ ID NO: 13). In some approaches, the promoter is an inducible promoter. In some approaches, the promoter is a constitutive promoter. The promoters may be naturally occurring promoters or hybrid promoters that combine elements of more than one promoter. In some approaches, the promoter is a tissue-specific promoter. Generally, the promoters are shortened versions of the human endogenous RPE-specific promoter sequences (e.g. RPE65-5022 nucleotides and BEST1-5479 nucleotides). Non-limiting examples of RPE-specific promoters include BEST1-EP-454; RPE65-EP-415; smCBA; CBA; RPE65-EP-419; sctmCBA; or VM D2, as described in International Patent Publication WO2020019002. Other promoters or modified promoters—including natural and synthetic—may also be used for controlling expression of the therapeutic products disclosed herein include, but are not limited to UBC, GUSB, NSE, synapsin, MeCP2, GFAP, PAI1, ICAM, fit-1, and CFTR (see Papadakis et al 2004; PROMOTERS AND CONTROL ELEMENTS: DESIGNING EXPRESSION CASSETTES FOR GENE THERAPY in Current Gene Therapy, 2004, 4, 89-113; Gray & Samulski 2011; VECTOR DESIGN AND CONSIDERATIONS FOR CNS APPLICATIONS in Gene Vector Design and Application to Treat Nervous System Disorders, ed. J. Glorioso (Washington, DC: Society for Neuroscience), 1-9.; Trapani et al 2014; VECTOR PLATFORMS FOR GENE THERAPY OF INHERITED RETINOPATHIES Progress in Retinal and Eye Research 43 (2014) 108e128; Powell and Gray 2015). VIRAL EXPRESSION CASSETTE ELEMENTS TO ENHANCE TRANSGENE TARGET SPECIFICITY AND EXPRESSION IN GENE THERAPY Discov. Med. 2015 January; 19(102): 49-57, each incorporated herein by reference. Additional promoters that may also be used include the early and late promoters of SV40, tet promoter, adenovirus or cytomegalovirus immediate early promoter, RSV promoters, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof.

The exogenous HTRA1 gene or transgene may also be under the control of other regulatory sequences such as enhancer or activator sequences, leader or signal sequences, ribosomal binding sites, transcription start and termination sequences, and polyadenylation sequence. Enhancers that may be used in approaches of the invention include but are not limited to: an SV40 enhancer, a cytomegalovirus (CMV) enhancer, an elongation factor 1 (EF1) enhancer, yeast enhancers, viral gene enhancers, and the like. Termination control region may comprise or be derived from a synthetic sequence, synthetic polyadenylation signal, an SV40 late polyadenylation signal, an SV40 polyadenylation signal, a bovine growth hormone (BGH) polyadenylation signal, viral terminator sequences, or the like.

Exemplary promoter and enhancer nucleotide sequences are provided as SEQ ID NOs: 6, 11, 12, and 13 (“promoter sequences”). It will be understood by those of skill in the art that regulatory (promoter/enhancer) sequences can tolerate a certain degree of variation whilst retaining the regulatory property. In certain approaches described herein in which a promoter/enhancer is called out, a substantially identical sequence (e.g., a sequence with at least about 90% identity, preferably at least about 91%, 92%, 93%, 94%, 95%, 96%, 97%, 98%, 99% nucleotide identity over the entire promoter/enhancer sequence) is contemplated as a suitable substitute for the called-out sequence.

### 7. Delivery of CRISPR, CRISPRa, Endonucleases, Repair Templates and Other Components

Systems (“delivery systems”) for delivering proteins and nucleic acids, including CRISPR elements, are well known in the art. These systems may be used to deliver Cas proteins (with or without nuclease activity), Cas nickase, sgRNA or other guide RNAs, Cas-transcriptional activator fusion proteins, HTRA1 protein-encoding sequence, template repair sequences, and the like to cells (e.g., RPE, photoreceptor and horizontal cells). The material delivered to a cell is sometimes referred to herein as a “transgene” or “cargo.” Hageman, G and Richards, B., International Patent Publication WO2020019002, and Yanik et al., 2017, In vivo genome editing as a potential treatment strategy for inherited retinal dystrophies Progress in Retinal and Eye Research 56:1-18 described methods for delivering components to cells in the eye for transgene expression, gene repair, gene activation, and the like, which may be adopted to the present invention. In some approaches, the delivery is by a virus e.g., lentivirus, adeno-associated virus (AAV), as described in Byrne et al., Methods Enzymol. 546, 119-38 (2014); Cong et al., Science (80). 339, 819-823; Hirsch et al., Mol. Ther. 18, 6-8 (2010).

In some approaches, the cargo (e.g., HTRA1 transgene) is delivered using a rAAV2 expression vector. In one approach, the transgene or other components (e.g., the exogenous HTRA1 gene, or the recombinant nucleic acid the expression of which in the RPE cells can increase the expression of the endogenous HTRA1) is delivered to the RPE using an rAAV2 system that is capable of transducing RPE cells at high efficiency. In addition to AAV2, other adeno-associated virus-based vectors include AAV1, AAV2, AAV3, AAV4, AAV5, AAV6, AAV7, AAV8, AAV9, AAV10, AAV11, and pseudotyped AAV. Retrovirus, lentivirus, adenovirus, baculovirus and others can also be employed, as disclosed in Lau and Suh (2017) doi: 10.12688/f1000research.11243.1, the relevant disclosure thereof is herein incorporated by reference.

For packaging a transgene into AAV vectors, the ITRs are the only AAV components required in cis in the same construct as the transgene. The cap and rep genes can be supplied in trans. Accordingly, DNA constructs can be designed so that the AAV ITRs flank the coding sequence for the anti-pathogen construct (or subunits thereof, or subunits thereof fused to a dimerizable domain which is part of a regulatable promoter), thus defining the region to be amplified and packaged—the only design constraint being the upper limit of the size of the DNA to be packaged (approximately 4.5 kb).

In addition to AAV vectors, other viral vectors that may be used include, but are not limited to, retroviruses, adenoviruses (AdV), lentiviruses, pox viruses, alphaviruses, and herpes viruses

Viral vectors (e.g., rAAV2, lentiviral vectors) carrying expression cassettes with HTRA1 transgenes or other cargo may be produced, collected and purified using art-known methods (including methods described in publications cited herein). For AAV methods see Zolotukin et al., 2002, Production And Purification Of Serotype 1, 2, And 5 Recombinant Adeno-Associated Viral Vectors” Methods 28:158-167, incorporated by reference, and, Aponte-Ubillus et al., 2018; Naso et al., 2017; and Penaud-Budloo et al., 2018; all incorporated by reference and cited above. Also see For general reviews related to gene therapy, including descriptions of expression and delivery systems see Moore et al., 2017, “Gene Therapy For Age-Related Macular Degeneration” Expert Opinion on Biological Therapy 17:10: 1235-1244; Aponte-Ubillus et al., 2018, “Molecular Design For Recombinant Adeno-Associated Virus (Raav) Vector Production” Applied microbiology and biotechnology 102.3:1045-1054; Ochakovski et al., 2017, “Retinal Gene Therapy: Surgical Vector Delivery In The Translation To Clinical Trials” Frontiers in Neuroscience 11; Schon et al., 2015, “Retinal Gene Delivery By Adeno-Associated Virus (Aav) Vectors: Strategies And Appucations” European Journal of Pharmaceutics and Biopharmaceutics 95:343-352; Naso et al., 2017, “Adeno-Associated Virus (Aav) As A Vector For Gene Therapy” BioDrugs 31:317; Dunbar et al., 2018, “Gene Therapy Comes Of Age” Science 359:6372; Penaud-Budloo et al., 2018., “Pharmacology of Recombinant Adeno-Associated Virus Production” Molecular Therapy: Methods & Clinical Development 8:166-180; each of which is incorporated by reference for all purposes.

Non-viral vectors or methods can also be used to deliver cargo. These include delivery using virus-like particles (VLPs), administration using cationic liposomes, cell-derived nanovesicles, direct nucleic acid injection, hydrodynamic injection, use of nucleic acid condensing peptides and non-peptides, cationic liposomes and encapsulation in liposomes. In one approach, virus-like particles (VLP's) are used to deliver cargo. The VLP comprises an engineered version of a viral vector, where nucleic acid or non-nucleic acid cargo are packaged into VLPs through alternative mechanisms (e.g., mRNA recruitment, protein fusions, protein-protein binding). See Itaka and Kataoka, 2009, “Recent development of nonviral gene delivery systems with virus-like structures and mechanisms,” Eur J Pharma and Biopharma 71:475-483; and Keeler et al., 2017, “Gene Therapy 2017: Progress and Future Directions” Clin. Transl. Sci. (2017) 10, 242-248, incorporated by reference.

### 8. Other Therapeutics for Increasing HTRA1 Expression or Activity

8.1 Cell Therapy

In one approach stem cells (e.g., iPSCs) are modified in vivo and ex vivo for transplantation into the RPE (see Peddle et al., “CRISPR Interference—Potential Application in Retinal Disease”, Int. J. Mol. Sci., 2020 21:1-14.

8.2 Other Agents

In some approaches, the method comprises treating the patient with a small molecule compound that can increase expression of HTRA1. A small molecule compound disclosed herein refers to an organic compound, typically with a molecular weight less than 5,000 daltons, less than 1,000 daltons, less than 900 daltons, or less than 800 daltons. The method of treating, preventing development, slowing progression of, reversing or ameliorating symptoms and signs of Chr 10 AMD may comprise administering a small molecule compound to the patient wherein the agent increases expression of the HTRA1. Exemplary compounds include Trichostatin A (TSA), an inhibitor of class I and class II histone deacetylases, which was shown to increase mRNA expression of HTRA1 by about two-fold. Wang et al., Plos|One 2012, https://doi.org/10.1371/journal.pone.0039446.

In some cases, suitable compounds can be identified by screening a library of compound using a quantitative in vitro transcription assay. An in vitro transcription assay can be in a cell-free system, which includes a plasmid comprising a HTRA1 and regulatory elements. In some approaches, identifying an agent that can increase expression of HTRA1 comprises contacting a library of agents with cells that express HTRA1, and measuring the expression level or activity of HTRA1, and selecting the agents that increase the HTRA1 expression or activity. A library of compounds are screened and transcription of HTRA1 can be measured using means well known in the art. For example, transcription can be carried out in the presence of radiolabelled or fluorescently labelled nucleotides, and the labelled transcripts can be precipitated on a gel, separated by electrophoresis, and then quantified. Alternatively, HTRA1 mRNA can be measured by quantitative RT-PCR or digital PCR. Agents that increase the transcription of HTRA1 are then selected and tested. Methods for selecting agents (including compounds or peptides) that can activate transcription are well known in the art, for example, as described in U.S. Pat. No. 6,174,722, the entire content of which is incorporated herein by reference.

Methods for constructing libraries that can be used for screening agents that can activate HTRA1 transcription are also well known. For example, combinatorial libraries can be produced for many types of compound that can be synthesized in a step-by-step fashion. Large combinatorial libraries of compounds can be constructed by the encoded synthetic libraries (ESL) method described in WO 95/12608, WO 93/06121, WO 94/08051, WO 95/35503 and WO 95/30642. Peptide libraries can also be generated by phage display methods (see, e.g., Devlin, WO 91/18980). Libraries of natural compounds in the form of bacterial, fungal, plant and animal extracts can be obtained from commercial sources or collected in the field. Known pharmacological agents can be subject to directed or random chemical modifications, such as acylation, alkylation, esterification, amidification to produce structural analogs.

In some approaches, the test agents can be naturally occurring proteins or their fragments. The test agents can also be peptides, e.g., peptides of from about 5 to about 30 amino acids, with from about 5 to about 20 amino acids being preferred, and from about 7 to about 15 being particularly preferred. The peptides can be digests of naturally occurring proteins, random peptides, or “biased” random peptides. The test agents can also be nucleic acids of various length and sequences.

In some approaches, agents that can be used to treat, prevent development of, slow progression of, reverse or ameliorate symptoms and signs of Chr 10 AMD can be identified by contacting an agent with a HTRA1 promoter that is operably linked to a reporter gene, and selecting the agent based on its ability to promote the expression of the reporter gene.

### 9. Patient Population

9.1 Patient Population

The compositions and methods of the invention find particular use for treatment of subjects with, or at risk of developing, Chromosome 10 AMD. As noted above, Chr 10 AMD is known to be associated with genetic lesions in chromosomal region 10q26, which harbors the ARMS2 and HTRA1 genes. See Hageman et al, 2015, “METHODS OF PREDICTING THE DEVELOPMENT OF AMD BASED ON CHROMOSOME 1 AND CHROMOSOME 10” US Pat. Pub. 2015/0211065, incorporated herein by reference. Patients with Chr 10 AMD can be identified based on disease manifestations and/or based on genotypes. In one approach, candidates for treatment exhibit a Chr 10 AMD clinical phenotype. Thus, in one approach the subject receiving treatment exhibits a Chr 10 clinical phenotype. In one approach the subject receiving treatment carries one or two chromosome 10 risk alleles. In one approach the subject receiving treatment carries one the risk alleles on one or two copies of chromosome 10 risk alleles. In one approach the subject has a Chr 10 AMD clinical phenotype or genotype and does not have any chromosome 1 risk alleles. Chromosome 1 risk alleles for AMD include rs529825, rs800292, rs3766404, rs1061147, rs203674, rs0161170, rs2274700, rs375046, rs9427661, rs9427662 and rs12097550.

Chr 10 AMD patients display primarily classic choroidal neovascularization (CNV), rarely occult CNV and retinal angiomatous proliferation (RAP), often resulting in severe, rapid visual loss. Chr 10 AMD patients typically have fewer drusen than Chr 1 AMD patients, intra-retinal fluid (cysts), a fast geographic atrophy (GA) growth rate, and retinal/choroidal thinning. Chr 10 AMD patients also have reduced retinal and choroidal (including the choriocapillaris) vascular densities.

In some aspects, enhancing HTRA1 expression is achieved by gene therapy. In some approaches, enhancing HTRA1 is achieved by administering an agent, e.g. a small molecule compound, a peptide, or a nucleic acid, to upregulate the transcription of the endogenous HTRA1 gene. Gene therapy to upregulate the transcription of HTRA1 can be performed in different ways. In some approaches the gene therapy upregulates the endogenous HTRA1 expression (mRNA or protein expression). In some approaches, the gene therapy introduces an exogenous HTRA1 gene, which is expressed in RPE cells. In some approaches, the gene therapy methods are used to convert Ch10 risk alleles in the cell genome to corresponding non-risk alleles, as described below.

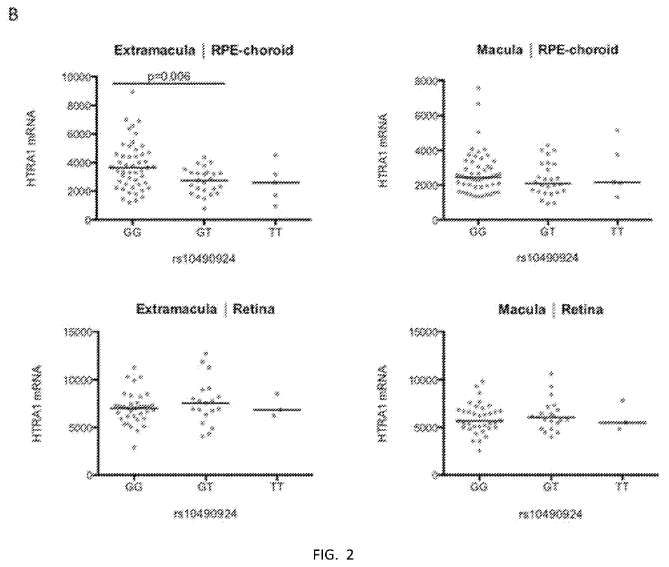

As described below, individuals having Chr 10 AMD often carry one or more risk alleles associated with development of the disease (“Chr 10 risk alleles”). See Examples 1 and 3. The inventors have discovered, surprisingly, that Chr 10 AMD patients have decreased HTRA1 mRNA expression specifically in the RPE relative to healthy or lower risk controls (FIG. 2). Further, the inventors have identified a genomic region responsible for decreased HTRA1, which corresponds to the region associated with increased AMD risk.

Patients homozygous for risk alleles are shown to have decreased HTRA1 protein in RPE cells, which may also affect the HTRA1 levels in the interface between RPE and Bruch's membrane (the “sub-RPE space”). It is believed that the decrease in HTRA1 expression, for example, the decrease in HTRA1 expression in the sub-RPE space, contributes to the development of Chr 10 AMD. HTRA1 is a serine protease and can degrade extracellular matrix (ECM) proteins that are highly enriched in the sub-RPE space. Without intending to be bound by a particular theory or mechanism, the reduction in HTRA1 expression may impair the processing, maintenance or turnover of ECM proteins, which results in accumulation of damaged, misfolded and/or aggregated proteins. Accumulation of these proteins, in turn, may disrupt attachment of the RPE tissue to its basal lamina and/or to Bruch's membrane, resulting in a loss of the blood-retina barrier and contributing the development of AMD. Table 4 shows odd's ratios and a p-value for association of certain phenotypes with Chr10 risk. Both chroidal fibrosis and basal laminar deposits (BLDs) have been observed in eyes from donors homozygous for chromosome 10 risk alleles. BLDs are abnormal extracellular material lying between the RPE plasma membrane and the basal lamina, and has been previously shown as strongly associated with late-stage AMD. Further, mutations in ECM structural proteins that result in their mis-regulation lead to AMD-like diseases including L-ORD (C1qTNF5), Sorsby Fundus Dystrophy (TIMP3), Ehlers Danlos Syndrome type VI (PLOD1) or Doyne's Honeycomb Retinal Dystrophy (EFEMP1). See Hayward et al., Hum. Mol. Genet. 12:2657-67 (2003); Weber et al., Nat. Genet 8 (4):352-6; and Marmorstein et al., PNAS. 99 (20): 13067-72. These evidence suggest that alterations of the ECM and the formation of basal laminar deposits share a common etiology and that the decreased expression of HTRA1 results in accumulation of damaged, misfolded and aggregated proteins and the development of AMD.

TABLE 4
Histological Associations in Chr10 AMD

| Feature | Region | OR(95% CI)¹ | | p-value¹ |
|---|---|---|---|---|
| Choriocapillary ghosts | Macular | 2.35 | (1.54-3.57) | <0.0001 |
| Bruch's BLDs | Macular | 058 | (0.41-0.83) | 0.0030 |
| Chroidal fibrosis | Extramacular | 1.59 | (1.15-2.20) | 0.0053 |
| Basal laminar | Macular | 2.00 | (1.20-3.32) | 0.0078 |
| deposits (BLDs) | | | | |
| Thickened Bruch's | Macular | 1.67 | (1.12-2.49) | 0.0113 |
| membrane | | | | |
| Basal laminar | Extramacular | 1.57 | (1.06-2.34) | 0.0256 |
| deposits (BLDs) | | | | |
| Chroidal fibrosis | Macular | 1.43 | (1.03-1.99) | 0.0316 |

¹Calculated using multivariate regression model adjusting for gender and age.

Accordingly, in some aspects, the disclosure provides methods of increasing HTRA1 expression in RPE cells to prevent or slow Chr 10 AMD development. The HTRA1 therapy can be administered after the patient has been identified as having the risk of developing Chr 10 AMD based on genetic profiles, and in some cases before manifestation of any clinical symptoms of AMD. This early intervention can avoid extensive tissue damage associated with AMD. The method disclosed herein requires minimal surgery, typically only once or twice during the patient's lifetime, thus minimizing the discomfort and adverse effect associated with repeated surgeries that are required for conventional therapies.

9.1.1 Selection of Subjects for Treatment

In some approaches, patients are selected for treatment based on a clinical phenotypes or genetic factors for treatment. In some approaches, they are assessed by genotyping to determine their individual genetics (e.g., by assess the presence of the Chr 10 risk alleles as disclosed above) and associated risk of disease. In addition, they may be assessed via a clinical exam, including but not limited to the following: imaging and morphological assessments (for example and including but not limited to, color fundus photography, SD-OCT, OCT-A, indocyanine green angiography, fluorescein angiography and confocal scanning laser ophthalmoscopy (for example Heidelberg Spectralis system), including near infrared reflectance (NIR), blue-light autofluorescence, green-light autofluorescence; and functional testing (for example and including but not limited to visual acuity, best corrected visual acuity (BCVA using ETDRS chart), Low luminance BCVA (LLVA, using neutral density filter with ETDRS chart), reading speed (monocular/binocular), microperimetry (MAIA) including fixation stability, dark-adapted microperimetry (S-MAIA), scotopic and mesopic microperimetry sensitivity, visual evoked potential (VEP) assessment and multifocal ERG.

Additional indicators include a combination of morphological and functional information (vision, reading speed, low light vision, fixation, electroretinogram, etc.).

Additionally, patients may be assessed based on a number of phenotypes and biomarkers. Administering the therapies disclosed here increases HTRA1 expression and also provides benefit to patients when administered in particular phenotypic windows defined by changes in the anatomy of the eye and appearance or changes in levels of certain biomarkers including, without limitation: having few drusen (drusen are small and hard), intra-retinal fluid (cysts), a fast GA growth rate, and retinal/choroidal thinning.

In one aspect, administration of the therapeutic agent disclosed herein at a very early stage of progression of Chr 10 AMD may provide superior therapeutic benefit. For example, treatment of patients prior to the appearance of signs or symptoms of Chr 10 AMD (e.g., no presence of any of the following symptoms: drusen, intra-retinal fluid (cysts), a fast GA growth rate, and retinal/choroidal thinning), particularly patients at high genetic risk by having one or more of the Chr 10 risk alleles. Thus, provided herein are methods and compositions for preventing development of Chr 10 AMD, as well as slowing progression of, reversing or ameliorating symptoms and signs of Chr 10 AMD. In some approaches, the patient has no symptoms of AMD (i.e. asymptomatic). In some approaches, at the time of first administration of the therapeutic agent, the patient does not exhibit any of the clinical phenotypes of Chr 10 AMD.

In some approaches, the patient has a combination of both Chr 1 and Chr 10, Chr1 and other minor AMD-associated genes (C3, CFB, C2, etc.), or a combination of all, and the patient is treated with the therapy of the present invention to treat, prevent or slow progression of symptoms and signs of Chr 10 AMD, and a second agent to treat, prevent or slow progression of Chr 1 AMD.

9.2 Genetic Factors Associated with Reduced HTRA1 Expression and Chr 10 AMD

9.2.1 Chr 10 Risk Alleles

In addition to clinical phenotypes, an individual may be also identified, based on genetic factors alone, as being at elevated risk for developing Chr 10 AMD.

Accordingly, the methods and compositions disclosed herein can be used to treat patients having these risk alleles with an agent that increases HTRA1 expression in the RPE cells of the subject, thereby treating, preventing the development, slowing the progression, or reversing or ameliorating symptoms and signs of Chr 10 AMD.

As described in the Examples, the region associated with decreased HTRA1 mRNA may be located in an upstream regulatory region of HTRA1, between and including, rs11200632 and rs3750846 (the “4 kb AMD risk region”). Exemplary Chr 10 risk alleles are located at rs11200632 (the risk allele is G), rs11200633 (the risk allele is T), rs61871746 (the risk allele is C), rs61871747 (the risk allele is T), rs10490924 (the risk allele is T), rs36212731 (the risk allele is T), rs36212732 (the risk allele is G), rs36212733 (the risk allele is C), rs3750848 (the risk allele is G), rs3750847 (the risk allele is T), and rs3750846 (the risk allele is C). Table 5 shows the risk alleles (top) and non-risk alleles (bottom) at these polymorphic sites. A complete list of SNPs in perfect LD (r²=1) or very high LD with rs10490924 that are within the 4 kb risk region are shown in Table 5. The methods and compositions disclosed herein can be used to treat patients having one or more of these risk alleles.

TABLE 5
Variants (SNPs) and LD Within the 4 kb Regulatory Region

| Variant | Location Hg38 | r² | D′ | Gene | non-risk/risk |
|---|---|---|---|---|---|
| rs11200632 | 10:122452020 | 1 | 1 | — | A/G |
| rs11200633 | 10:122452080 | 1 | 1 | — | C/T |
| rs61871746 | 10:122453397 | 1 | 1 | — | T/C |
| rs61871747 | 10:122453530 | 1 | 1 | — | C/T |
| rs758050792 | 10:122453628- | 0.886 | 1 | — | —/AAA |
| | 122453630 | | | | |
| rs370728615 | 10:122454155- | 0.938 | 1 | — | —/CAACAACAAC |
| | 122454164 | | | | |
| rs201396317 | 10:122454158 | 0.938 | 1 | — | C/A |
| rs199637836 | 10:122454161 | 0.938 | 1 | — | C/A |
| rs11200634 | 10:122454164 | 0.938 | 1 | — | C/A |
| rs75431719 | 10:122454172 | 0.938 | 1 | — | C/A |
| rs10490924 | 10:122454932 | | | ARMS2 | G/T |
| | | | | ex1/LOC105378525 | |
| | | | | Alt Ex3 | |
| rs144224550 | 10:122455084- | 1 | 1 | ARMS2 | —/GT |
| | 122455085 | | | | |
| rs36212731 | 10:122455460 | 1 | 1 | ARMS2 | G/T |
| rs36212732 | 10:122455682 | 1 | 1 | ARMS2 | A/G |
| rs36212733 | 10:122455695 | 1 | 1 | ARMS2 | T/C |
| rs3750848 | 10:122455799 | 1 | 1 | ARMS2 | T/G |
| rs3750847 | 10:122455905 | 1 | 1 | ARMS2 | C/T |
| rs3750846 | 10:122456049 | 1 | 1 | ARMS2 | T/C |

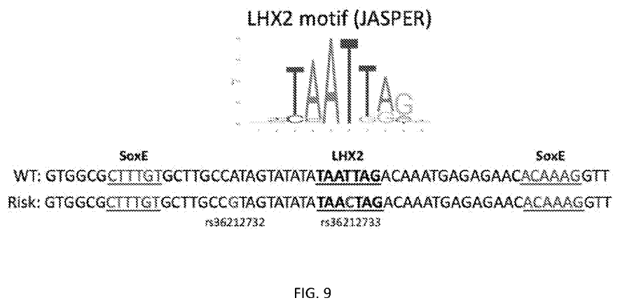

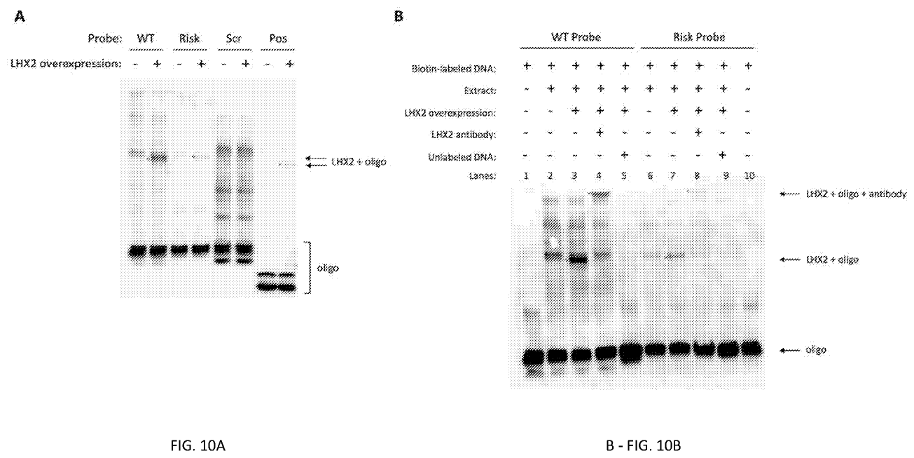

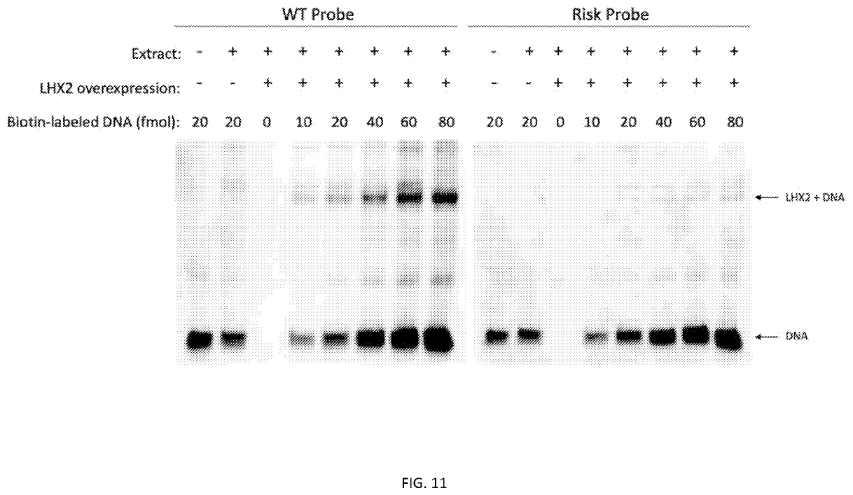

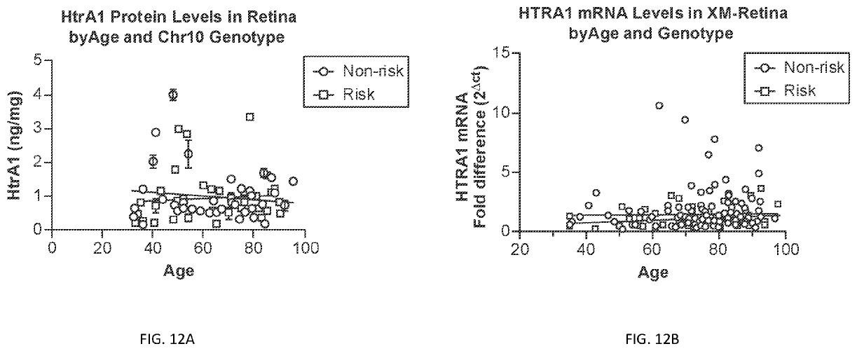

In some approaches, individuals who can benefit from the therapy disclosed herein may carry one or more risk alleles that results in decreased binding to one or more transcriptional activators and decreased transcription of HTRA1 gene. As shown in the examples, the inventors have discovered a 2 kb region located at Chr10:122454508-Chr10:122456564 (“the 2 kb risk region”), which is transcriptionally active in RPE cells and is believed to be responsible for binding transcriptional activators that can activate HTRA1 transcription. This 2 kb region comprises epigenetic markers of an active transcription enhancer element, including H3K4 mono-methylation and H3K27 acetylation. For example, LHX2 binds to a sequence motif within this region, which has a nucleotide sequence TTGCCATAGTATATATAATTAGACAAAT (comprising the non-risk allele T, underlined, at rs36212733). LHX2 binds poorly to TTGCCGTAGTATATATAACTAGACAAAT (comprising a risk allele C, underlined, at rs36212733). See FIG. 9 (FIGS. 10 and 11). Accordingly in some approaches, the method disclosed herein comprises administering an agent to a patient, whose genomic DNA in this 2.0 kb region has reduced binding affinity to transcriptional activators for HTRA1, and administration of the agent increases HTRA1 expression in RPE cells. In some approaches, the patient showed reduced or complete loss of binding to LHX2. In some approaches, the patients may have a risk allele of C at rs36212733, (i.e., TTGCCATAGTATATATAACTAGACAAAT), which results in its loss of binding to LHX2. In some approaches, the method of treatment comprises administering an agent to the patient, wherein the agent increases HTRA1 expression in the RPE cells of the patient by promoting the binding of transcriptional activators (e.g., LHX2) to the transcriptional regulatory region of HTRA1.

9.2.2 IncSCTM1 Expression is Inversely Related to HTRA1 Expression

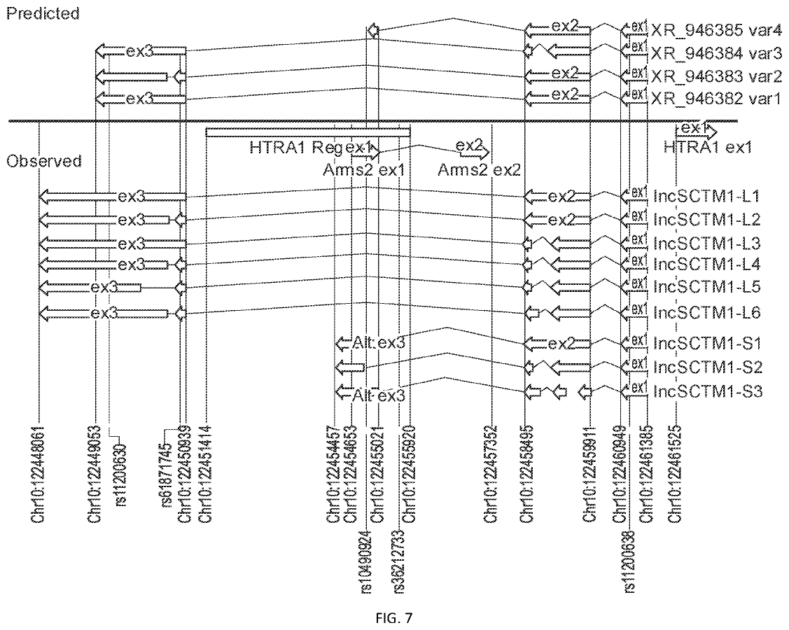

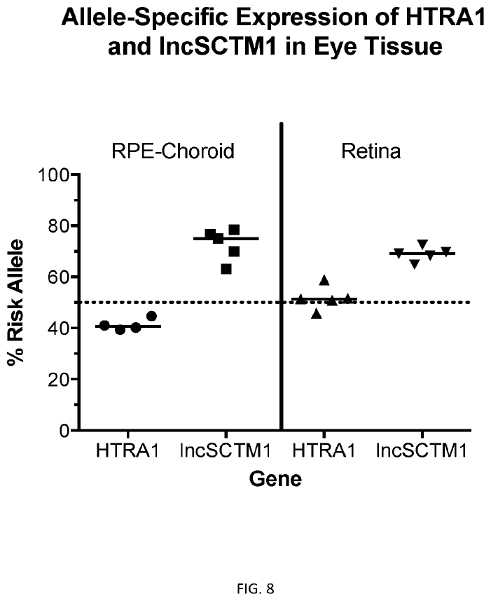

In some approaches, the individual who can benefit from the treatment disclosed herein showed increased allele-specific expression of a noncoding RNA, IncSCTM1 (also referred to as LOC105378525), or its isoforms, as compared to controls. IncSCTM1 is transcribed from a DNA sequence that shares the same LD block with HTRA1. As shown in FIG. 7 and the examples, IncSCTM1 shares a divergent promoter with HTRA1, and is transcribed in the anti-sense orientation from the HTRA1 promoter. The allele-specific expression of IncSCTM1 is inversely related to the allele-specific expression of HTRA1. In heterozygous patients, the mRNA levels of the risk allele of IncSCTM1 (e.g., rs11200638) is higher level than the mRNA of the non-risk allele of IncSCTM1. In contrast, the mRNA levels of the risk allele of HTRA1 are lower than the mRNA levels of the non-risk allele (FIG. 8).

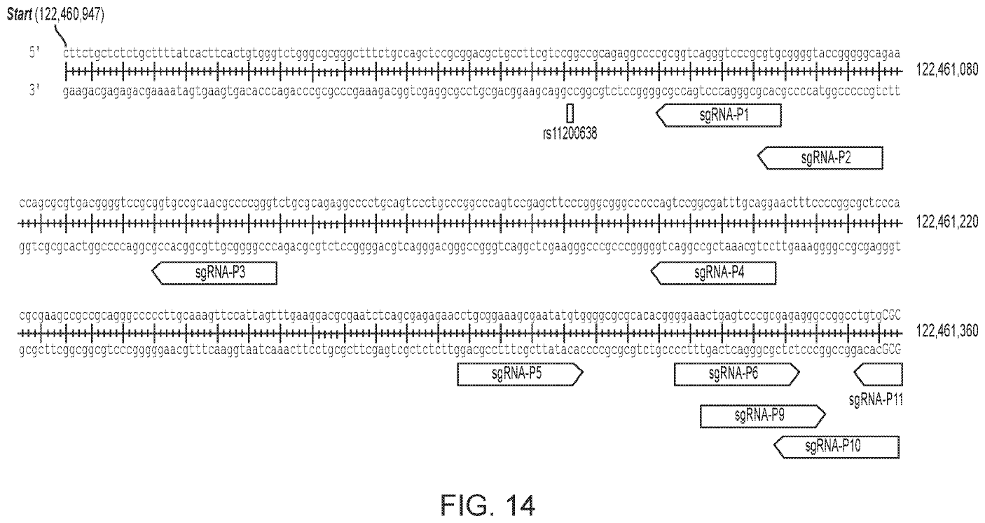

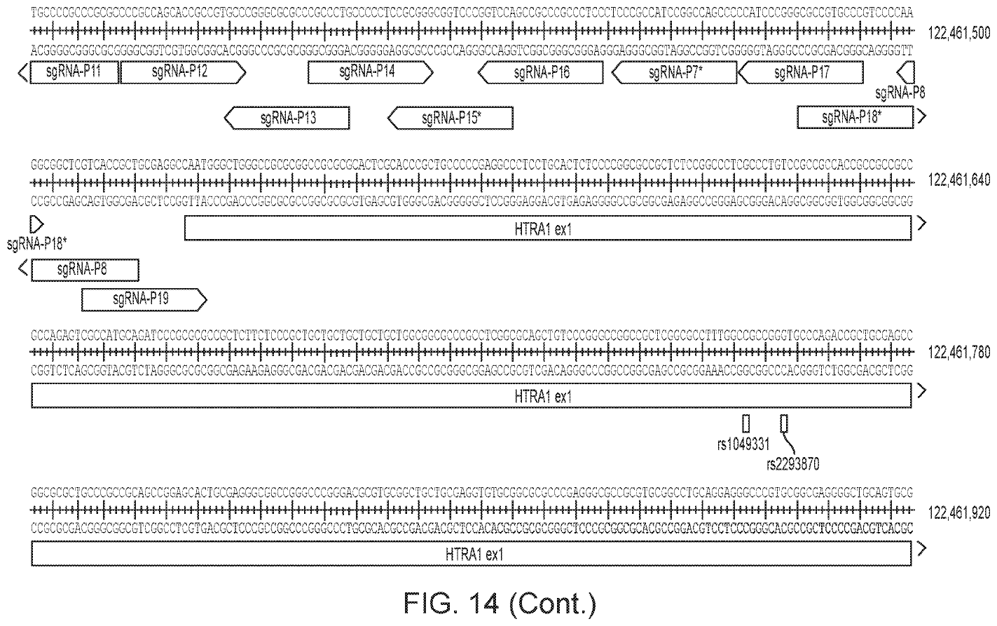

In some approaches, a patient who may benefit from the therapy disclosed herein carries one or more splice forms of IncSCTM1, which are correlated with decreased expression of HTRA1. As shown in the examples, IncSCTM1 exists as different splice variants (FIG. 7) and expressed in different eye tissues (FIG. 14).

In some approaches, the method and compositions disclosed herein can be used to treat Chr 10 AMD in an individual who has one or more of the clinical phenotypes of Chr 10 AMD, e.g., having few drusen, intra-retinal fluid (cysts), exhibits a fast GA growth rate, retinal/choroidal thinning In some approaches, the patient has one or more Chr 10 risk alleles as disclosed above. In some approaches, the patient has both the one or more clinical phenotypes of Chr 10 AMD and one or more Chr 10 risk alleles.

In some cases, in addition to having the Chr 10 AMD clinical phenotypes and/or having Chr 10 risk alleles, the patient also has Chr 1 risk alleles and/or Chr 1-driven AMD clinical phenotypes. Chr 1 risk alleles/haplotypes. See U.S. Pat. No. 7,867,727, incorporated herein by reference. In some cases, the patient has no Chr 1 risk alleles/haplotypes for AMD.

### 10. Administration Methodology and Dose

As described above, aspects of the invention include methods of administering agents to either increase HTRA1 expression or edit the genomic regions to convert risk alleles to non-risk alleles to a subject in need of treatment. As such, aspects of the invention include contacting the subject with one or more therapeutic agents, e.g., a viral vector, a compound, a peptide, or a combination thereof as described above, under conditions by which delivery of the agent in the subject results in a beneficial effect on one or more aspects of the subject's health. The invention is not limited to a particular site or method of administration. For example, for illustration and not limitation, the agent may be administered by systemic administration (e.g., intravenous injection or infusion), local injection or infusion (e.g., subretinal, suprachoroidal, intravitreal, transscleral or other ocular), by use of an osmotic pump, by electroporation, by application (e.g., eye drops) and by other means. It is contemplated that transgenes of the invention may be introduced into, and expressed in, a variety of cell types including neural retinal cell types, such as rods, cones, RPE, and ganglion cells, ciliary epithelial, scleral, choroidal and other ocular cells.

The therapeutic agent disclosed herein may be suspended in a physiologically compatible carrier for administration to a human. Administration may be by an ocular or nonoccular (e.g., administered intravitreally, intravascularly, extraocularly) route. Suitable carriers may be readily selected by one of skill in the art in view of the route of delivery. For example, one suitable carrier includes saline, which may be formulated with a variety of buffering solutions (e.g., phosphate buffered saline).

10.1 Ocular Administration

10.1.1 Subretinal and Other Injection

Introduction of the therapeutic agent near the level of the RPE-choroid interface provides better control of HTRA1 regulation during early stages of Chr 10 AMD and prevents blindness associated with late stage geographic atrophy and choroidal neovascularization. In one approach, the agent is administered via subretinal injection by creating a bleb or blister under the retina to deliver the viral vector directly to the region of the retina impacted by the disease. One or more blebs can be created in one quadrant or multiple quadrants of the eye to ensure sufficient distribution of the therapeutic agent. See Xue et al., “TECHNIQUE OF RETINAL GENE THERAPY: DELIVERY OF VIRAL VECTOR INTO THE SUBRETINAL SPACE” Eye 31:1308-1316, 2017. Also see Moore et al. 2017, Ochakovski et al. 2017, Schön et al. 2015, supra.

In another approach, the agent is administered via a suprachoroidal injection, thereby accessing the basal surface of the RPE. See Ding et al., “AAV8-vectored suprachoroidal gene transfer produces widespread ocular transgene expression”, J Clin Invest 129(11):4901-4911, 2019. Also see Emami- and Yiu, Medical and Surgical Applications for the Suprachoroidal Space, Int Ophthalmol Clin 59(1):195-207, 2019. In another approach, the agent can be injected into the vitreous. This approach can be especially useful for getting agent to the RPE in Chr 10 AMD patients with geographic atrophy or CNV. This technique is well known to those skilled in the art. See Kansar et al., “Suprachoroidal Delivery of Viral and Nonviral Gene Therapy for Retinal Disease”, J Ocular Pharmacol Ther DOI: 10.1089/jop.2019.0126.2020.

Dose

It is to be noted that dosage values may vary with the nature of the product and the severity of the condition. It is to be further understood that for any particular subject, specific dosage regimens can be adjusted over time according to the individual need and the professional judgment of the person administering or supervising the administration of the compositions, and that dosage ranges set forth herein are exemplary only and are not intended to limit the scope or practice of the claimed composition.

The amount of agent administered will be an “effective amount” or a “therapeutically effective amount,” i.e., an amount that is effective, at dosages and for periods of time necessary, to achieve a desired result. A desired result would include an improvement in HTRA1 expression or activity in a target cell (e.g., an RPE cell) or a detectable improvement in a symptom associated with reduced HTRA1 expression, including without limitation an improvement in AMD symptoms or signs, preferably a statistically significant improvement. Alternatively, if the pharmaceutical composition is used prophylactically, a desired result would include a demonstrable prevention of one or more symptoms of Chr 10 AMD, including without limitation, a symptom or sign of AMD, preferably a statistically significant prevention. A therapeutically effective amount of such a composition may vary according to factors such as the disease state, age, sex, and weight of the individual, or the ability of the viral vector to elicit a desired response in the individual. Dosage regimens may be adjusted to provide the optimum response. A therapeutically effective amount is also one in which any toxic or detrimental effects of the agent, e.g., a viral vector, are outweighed by the therapeutically beneficial effects. The amount of viral vector in the composition may vary according to factors such as the disease state, age, sex, and weight of the individual.

Dosage regimens may be adjusted to provide the optimum therapeutic response. For example, a single bolus may be administered, several divided doses may be administered over time or the dose may be proportionally reduced or increased as indicated by the exigencies of the therapeutic situation. In the case the therapeutic agent is a AAV particle, a preferred human dosage may be 10⁸ to 10¹² AAV genomes per injection in a volume of 100-300 μl per subretinal bleb. More than one bleb may be created per eye. Multiple treatments may be required in any given individual over a lifetime.

### 11. Treatment Outcome

HTRA1 gene therapy in a suitable patient, including treatment of an individual at risk of developing AMD or in early stages of the disease, can stabilize, ameliorate or reverse a symptom or sign of AMD in the patient. For example and without limitation, providing an exogenous HTRA1 protein, introducing transcriptional activator to patients that are heterozygous or homozygous for a Chr 10 risk allele can stabilize and/or slow or even reverse the progression of the disease, as demonstrated by various ocular biomarkers. In one approach the primary desired treatment outcome in a patient treated with HTRA1 related therapy is detectable improvement in one or more of the symptoms associated with Chr 10 AMD, having drusen, intra-retinal fluid (cysts), a fast GA growth rate, and retinal/choroidal thinning, reduced retinal and choroidal vascular densities, choriocapillary ghosts (no flow on OCT-A) in the macular region, choroidal fibrosis in the macular region, choroidal fibrosis in the extramacular region, Bruch's basal laminar deposits (BLD) in the macular region, BLDs in the macular region, thickened Bruch's membrane in macular region, BLDs in the extramacular region.

The desired treatment outcome in a patient treated with HTRA1 related therapy can also be detectable improvement in one or more of the functional measures, including without limitation: visual acuity (Early Treatment Diabetic Retinopathy Study, or ETDRS); best corrected visual acuity (or BCVA); microperimetry (macular integrity assessment, or MAIA); dark adaptation; reading speed; visual evoked potential (VEP); and multifocal electroretinography (mfERG), are contemplated. Other biomarkers indicative of stabilization, slowing, or reversing AMD progression including without limitation: BCVA Change; Area of GA Change (square root transformation or otherwise); Fixation; Reading Speed; % New Areas of GA; Photoreceptor Height; Individual Druse Characteristics.

### 12. Pharmaceutical Compositions

Another aspect of the invention pertains to pharmaceutical compositions of the vectors of the invention. In one embodiment, the composition includes an effective amount of the agent and a pharmaceutically acceptable carrier. In some approaches, sterile injectable solutions can be prepared by incorporating a vector, e.g., a viral vector, in the required amount, optionally with a diluent or excipient suitable for injection into a human patient. Provided are unit dosage forms such as a single use, pre-filled syringes or other injection device, with e.g., sufficient AAV particles or compounds for a single administration to a patient. Any pharmaceutical preparation of this invention may be packaged with or accompanied by information about the preparation and its use for treating AMD.

### 13. Definitions & Conventions

Before the present invention is described in greater detail, it is to be understood that this invention is not limited to particular approaches described, as such may, of course, vary. It is also to be understood that the terminology used herein is for describing particular approaches only, and is not intended to be limiting, since the scope of the present invention will be limited only by the appended claims.

As used herein the term, “transgene”, used interchangeably with “exogenous gene”, refers to a recombinant polynucleotide construct that is introduced into a cell using a gene therapy vector, to result in expression.

As used herein, the term “native promoter” refers to a promoter that is naturally and/or originally present in a cell and it is typically designated for the expression of a particular gene. For example, SEQ ID NO: 13 is a native HTRA1 promoter. A non-native promoter of a gene is one that that is not naturally associated with the gene. For example, the VMD2 promoter (SEQ ID NO: 6) is not a native HTRA1 promoter.

As used herein, the term “native transcriptional activator” refers to a transcriptional activator that is naturally and/or originally present in a cell and it is typically designated for regulating the transcription of a particular gene. For example, LHX2 is a native transcriptional activator for HTRA1 promoter, while VP16 is not a native transcriptional activator for HTRA1.

As used herein, “gene therapy vector” refers to virus-derived sequence elements used to introduce a transgene into a cell.

As used herein, “a viral vector” refers to a gene therapy vector including capsid proteins, used to deliver a transgene to a cell.

As used herein, the terms “promoter” refers to a DNA sequence capable of controlling (e.g., increasing) the expression of a coding sequence or functional RNA. A promoter may include a minimal promoter (a short DNA sequence comprised of a TATA-box and other sequences that serve to specify the site of transcription initiation). An enhancer sequence (e.g., an upstream enhancer sequence) is a regulatory element that can interact with a promoter to control (e.g., increase) the expression of a coding sequence or functional RNA. As used herein, reference to a “promoter” may include an enhancer sequence.

Promoters and other regulatory sequences are “operably linked” to a transgene when they affect to the expression or stability of the transgene or a transgene product (e.g., mRNA or protein).

As used herein, the terms “introduce” or “introduced,” in the context of gene therapy refers to administering a composition comprising, e.g., a polynucleotide (DNA) encoding a HTRA1 polypeptide, a transcriptional activator that can increase expression of HTRA1, or a DNA endonuclease, under conditions in which polynucleotide enters cells and is expressed in the cells to produce proteins. Polynucleotides may be introduced as naked DNA, using a viral (e.g., AAV2) vector, using a non-viral vector system, of by other methods.

The term “corresponds to” and grammatical equivalents is used herein to refer to positions in similar or homologous protein or nucleotide sequences, whether the exact position is identical or different from the molecule to which the similarity or homology is measured. For example, given a first protein 100 residues in length and a second protein that that is identical to the first protein except for a deletion of 5 amino acids at the amino terminus, position 12 of the first protein will “correspond” to position 7 of the second protein.

“Adeno-associated virus 2 (AAV2)” and “recombinant Adeno-associated virus 2 (rAAV2) are used equivalently. Exemplary AAV2 vectors are derived from the adeno-associated virus 2 genome and are described extensively in the scientific literature. See, e.g., Srivastava, et al, 1983, J. Virol. 45:555-564, incorporated herein by reference and other references cited herein below.

“Lentivirus,” as used herein refers to a gene therapy vector (lentiviral vector) that may be used to transduce a transgene into a cell. See, e.g., Keeker et al., 2017, Clin Transl Sci. 10:242-248, incorporated herein by reference and other references cited herein below.

The terms “identical” or percent “identity,” in the context of two or more nucleic acids or polypeptide sequences, refer to two or more sequences or subsequences that are the same (“identical”) or have a specified percentage of amino acid residues or nucleotides that are the same (i.e., at least about 70% identity, at least about 75% identity, at least 80% identity, at least about 90% identity, preferably at least about 91%, 92%, 93%, 94%, 95%, 96%, 97%, 98%, 99%, or higher identity over the entire sequence of a specified region, when compared and aligned for maximum correspondence over a comparison window or designated region as measured by manual alignment and visual inspection or using a BLAST or BLAST 2.0 sequence comparison algorithms with default parameters described below (see, e.g., NCBI web site ncbi.nlm.nih.gov/BLAST/ or the like)). Such sequences are then said to be “substantially identical.”

The term “subject” or “patient” refers to a human or an animal (particularly a mammal) and other organisms that receive either prophylactic or therapeutic treatment. For example, a subject can be a non-human primate.

As described below, the preferred algorithms can account for gaps and the like. Preferably, identity exists over a region that is at least about 25 amino acids or nucleotides in length, or more preferably over a region that is 50-100 or more amino acids or nucleotides in length. For sequence comparison, typically one sequence acts as a reference sequence, to which test sequences are compared. In some approaches, a percentage identity is determined in relation to the full length of a reference sequence selected from SEQ ID NO: 1 (nucleotide sequence of HTRA1) or SEQ ID NO: 2 (amino acid sequence of HTRA1). When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters. A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)). An algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (www.ncbi.nlm.nih.gov/). This algorithm involves first identifying high scoring sequence pairs (HSPs) by identifying short words of length within the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs comprising them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4 and a comparison of both strands. For amino acid sequences, the BLASTP program uses as defaults a word length of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

“Drusen” are focal extracellular deposits comprising lipids, fluid, a variety of proteins including complement pathway-related proteins, located between the RPE basal lamina and Bruch's membrane. Drusen are visible ophthalmoscopically as white/yellow dots and can be detected using a variety of art-known methods including those described in Wu et al., 2015, “FUNDUS AUTOFLUORESCENCE CHARACTERISTICS OF NASCENT GEOGRAPHIC ATROPHY IN AGE-RELATED MACULAR DEGENERATION” Invest Ophthalmol Vis Sci. 56:1546-52 and in References 1-8 of that reference. As used herein, the terms “small drusen” and “small hard drusen” refer to distinct drusen with a diameter less than about 63 μm. The terms “large drusen,” “soft drusen,” and “large soft drusen” refer to drusen with a diameter greater than about 125 μm, which are often clustered. Drusen with a diameter between 63 and 125 μm can be referred to as “intermediate drusen.” Localized detachments of the RPE, typically referred to as pigment epithelial detachments (PEDs), are often called drusen.

“Haplotype” as used herein refers to a DNA sequence or a combination of DNA sequences present at various loci on a chromosome that are transmitted together; a haplotype may be one locus, several loci, or an entire chromosome depending on the number of recombination events that have occurred between a given set of loci.

The term “polymorphism” refers to the occurrence of one or more genetically determined alternative sequences or alleles in a population. A “polymorphic site” is the locus at which sequence divergence occurs. Polymorphic sites have at least one allele. A diallelic polymorphism has two alleles. A triallelic polymorphism has three alleles. Diploid organisms may be homozygous or heterozygous for allelic forms. A polymorphic site can be as small as one base pair. Examples of polymorphic sites include: restriction fragment length polymorphisms (RFLPs), variable number of tandem repeats (VNTRs), hypervariable regions, minisatellites, dinucleotide repeats, trinucleotide repeats, tetranucleotide repeats, and simple sequence repeats. As used herein, reference to a “polymorphism” can encompass a set of polymorphisms (i.e., a haplotype).

A “single nucleotide polymorphism (SNP)” can occur at a polymorphic site occupied by a single nucleotide, which is the site of variation between allelic sequences. The site can be preceded by and followed by highly conserved sequences of the allele. A SNP can arise due to substitution of one nucleotide for another at the polymorphic site. Replacement of one purine by another purine or one pyrimidine by another pyrimidine is called a transition. Replacement of a purine by a pyrimidine or vice versa is called a transversion. A synonymous SNP refers to a substitution of one nucleotide for another in the coding region that does not change the amino acid sequence of the encoded polypeptide. A non-synonymous SNP refers to a substitution of one nucleotide for another in the coding region that changes the amino acid sequence of the encoded polypeptide. A SNP may also arise from a deletion or an insertion of a nucleotide or nucleotides relative to a reference allele.

As used herein, “linkage disequilibrium” or “LD” is the non-random association of alleles at two or more loci, not necessarily on the same chromosome. It is not the same as linkage, which describes the association of two or more loci on a chromosome with limited recombination between them. Linkage disequilibrium describes a situation in which some combinations of alleles or genetic markers occur more or less frequently in a population than would be expected from a random formation of haplotypes from alleles based on their frequencies. Non-random associations between polymorphisms at different loci are measured by the degree of linkage disequilibrium (LD). The level of linkage disequilibrium can be influenced by a number of factors including genetic linkage, the rate of recombination, the rate of mutation, random drift, non-random mating, and population structure. “Linkage disequilibrium” or “allelic association” thus means the non-random association of a particular allele or genetic marker with another specific allele or genetic marker more frequently than expected by chance for any particular allele frequency in the population. A marker in linkage disequilibrium with an informative marker, such as one of the SNPs, haplotypes, or diplotypes described herein can be useful in detecting susceptibility to Chr 10-driven AMD.

“ARMS2” refers to the AMD susceptibility 2 gene. The ARMS2 gene consists of two exons and theoretically encodes a 107 amino acid protein with no homology to known protein motifs and no known functions. The expression and localization of ARMS2 protein within cells remains elusive and the use of poorly characterized antibodies has resulted in conflicting reports (Fritsche et al., 2008; Kanda et al., 2007; Kortvely et al, 2010; Wang et al., 2012). Furthermore, RNA-Seq analyses indicates that expression of ARMS2 mRNA is exceptionally low in most tissues (TPM <1.0) with the exception of testis (TPM is approximately 6.9) and placenta (qRT-PCR data not shown) (Lonsdale et al., 2013; The GTEx Consortium, 2015).

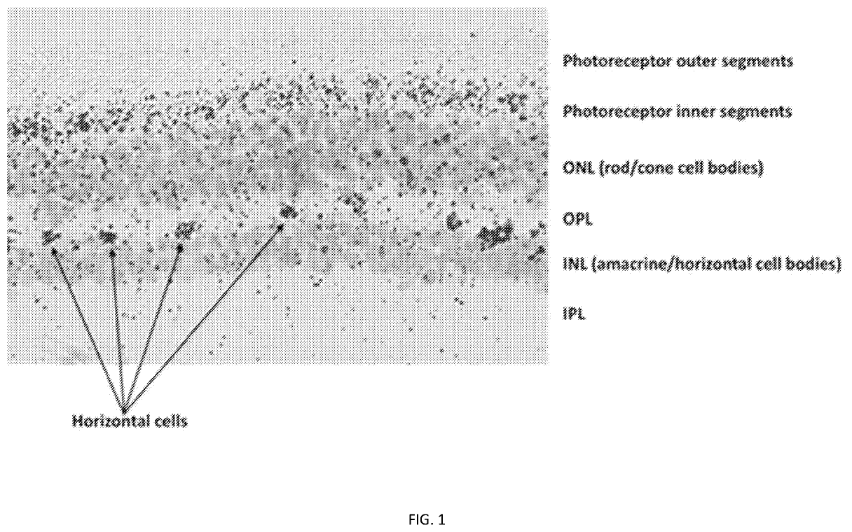

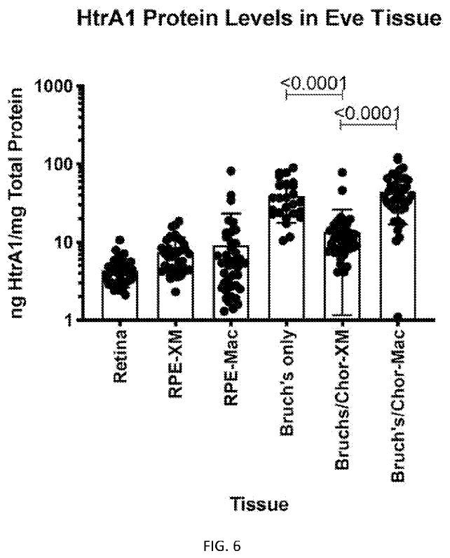

“HTRA1” refers to HtrA serine peptidase 1, the mRNA and the protein of which are represented by Gene Bank Accession Nos. NM_002775 and NP_002766, respectively. HTRA1 is ubiquitously expressed in nearly all cells and tissues examined and it is abundantly expressed in photoreceptors and horizontal cells of the retina, retinal pigmented epithelium (RPE) and various cell types in the choroid (FIG. 1). HTRA1 is enriched in extracts derived from Bruch's membrane and choroid as compared to retina and RPE (FIG. 6). HTRA1 acts as a secreted serine protease, and exists in solution primarily as an approximately 150 kDa trimer. HTRA1 is activated by an allosteric mechanism (Cabrera et al., 2017) and cleaves a variety of extracellular matrix proteins, proteoglycans, and many growth factors, such as TGFb, FGF and IGFBP. In a HTRA1 knockout mouse model, loss of HTRA1 resulted in an increase in many ECM proteins, including TIMP3, clusterin, elastin, vitronectin and fibulin 3 in the brain vessel proteome (Zellner et al., 2018). Loss of function mutations that impair HtrA1 protease activity or reduce mRNA expression can also cause CARASIL (cerebral autosomal recessive arteriopathy with subcortical infarcts and leukoencephalopathy) due to age-related vasculature defects in the brain (Hara et al., 2009; Fukutake, 2011).

The term “treatment” or any grammatical variation thereof (e.g., treat, treating, treatment, etc.), as used herein, includes but is not limited to, alleviating a symptom of a disease or condition; and/or reducing, suppressing, inhibiting, lessening, ameliorating or affecting the progression, severity, and/or scope of a disease or condition.

As used herein, the term “transcriptional regulatory region” refers to the HTRA1 promoter and the HTRA1 enhancer.

As used herein, the term “enhancer” refers to the HTRA1 2 kb region.

As used herein, the term “corresponding to” in the context of a sequence refers to the compliment, the RNA sequence that is equivalent to a DNA sequence but for the substitution of U for T, and the like as apparent from context.

As used herein “codon optimization” has its usual meaning in the art. Codon optimization can be used to increase the rate of translation or to produce recombinant RNA transcripts having desirable properties, such as a longer half-life and greater expression efficiency, as compared with transcripts produced using a non-optimized sequence. In some embodiments, the present disclosure provides HTRA1 coding sequences that have been engineered to to maximize expression efficiency. Methods for codon optimization are readily available, for example, optimizer, accessible free of charge at http://genomes.urv.es/OPTIMIZER, and GeneGPS® Expression Optimization Technology from DNA 2.0 (Newark, Calif.). In particular embodiments, the coding sequence is codon-optimized for expression in human using the OptimumGene™ algorithm from GenScript (Piscataway, N.J.).

As used herein a Cas protein that “lacks nuclease activity” has at least a 50% reduction in activity relative to the wild-type equivalent, sometimes at least an 80% reduction, sometimes at least an 95% reduction, and sometimes at least a 99% reduction.

As used herein the term “horizontal cells” have the normal meaning in the art. See, e.g., Poché et al., “Retinal horizontal cells: challenging paradigms of neural development and cancer biology”, Development, 2009, 136, Pages 2141-2151. “Horizontal cells” are laterally interconnecting neurons having their cell bodies in the inner nuclear layer of the retina of vertebrate eyes. They integrate and regulate the input from multiple photoreceptor cells.

As used herein the term “photoreceptor cells” are specialized neuroepithelial cells found in the outermost layer of the neural retina. They are capable of visual phototransduction and there are two major types, the rods which mediate scotopic vision and the cones which mediate photopic vision.

As used herein a “programmable” endonuclease is a nuclease that can be specifically targeted to a particular DNA sequence by selection of an associated molecule (e.g. gRNA for Cas proteins), fused protein sequence, or other means.

The following conventions are used herein. A DNA “target sequence” [A] is contiguous with a PAM [P] on the first DNA strand. The complement of the target sequence [C] is found on the complementary DNA strand. The guide sequence [G] of the gRNA hybridizes to, and is complementary to, the complement of the target, and has the sequence of the target [A], except that thymidine in DNA is replaced by uracil in RNA. The guide sequence of the gRNA can be produced by transcription from [C].

Unless otherwise indicated, nucleotide sequences are presented 5′ to 3′.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

### 14. Examples

14.1 Expression of HTRA1 is Reduced in RPE of Human Donors with Chromosome 10 Risk Alleles

This example shows that human donor eyes from individuals having AMD risk alleles had lower mRNA HTRA1 expression as compared to controls.

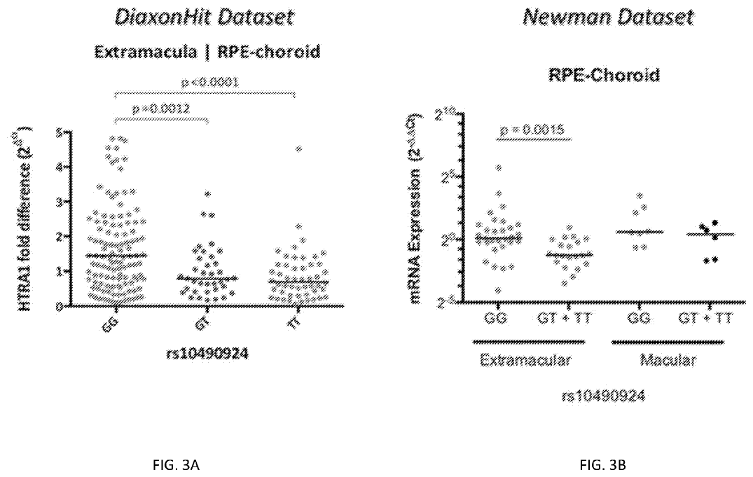

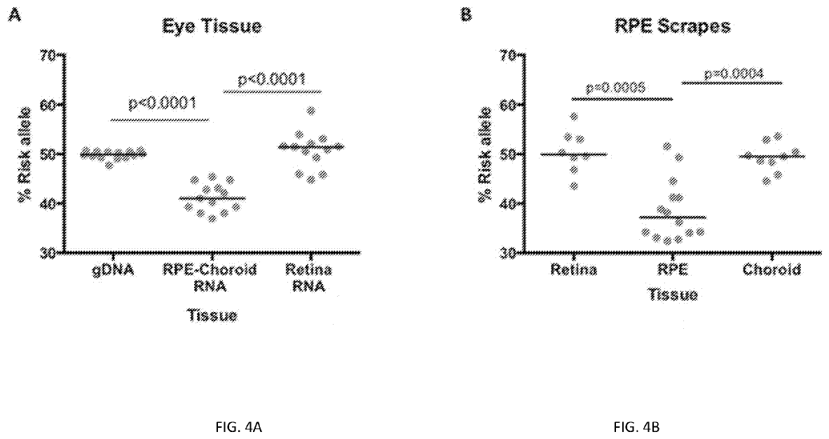

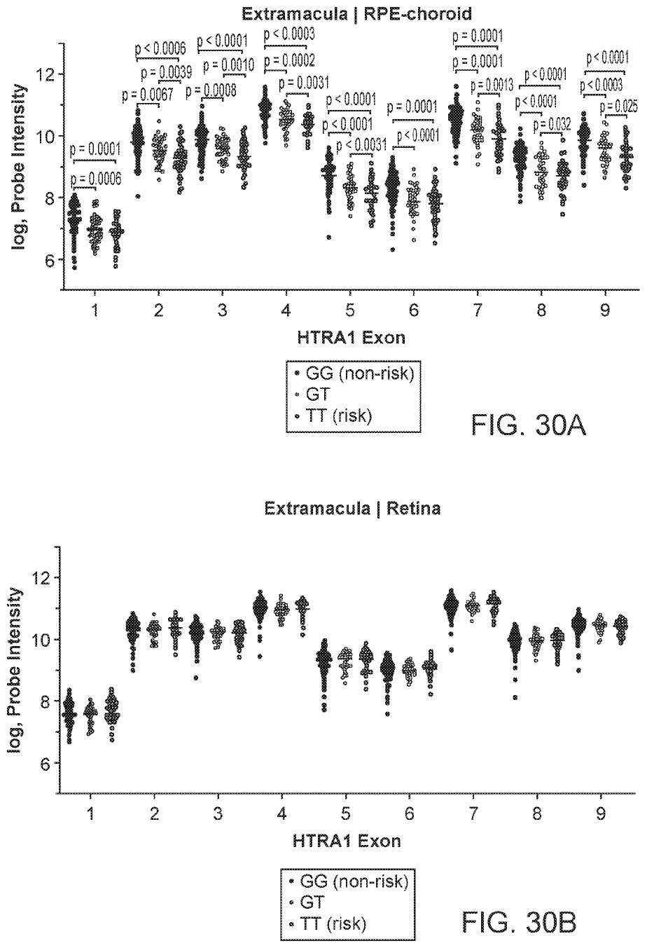

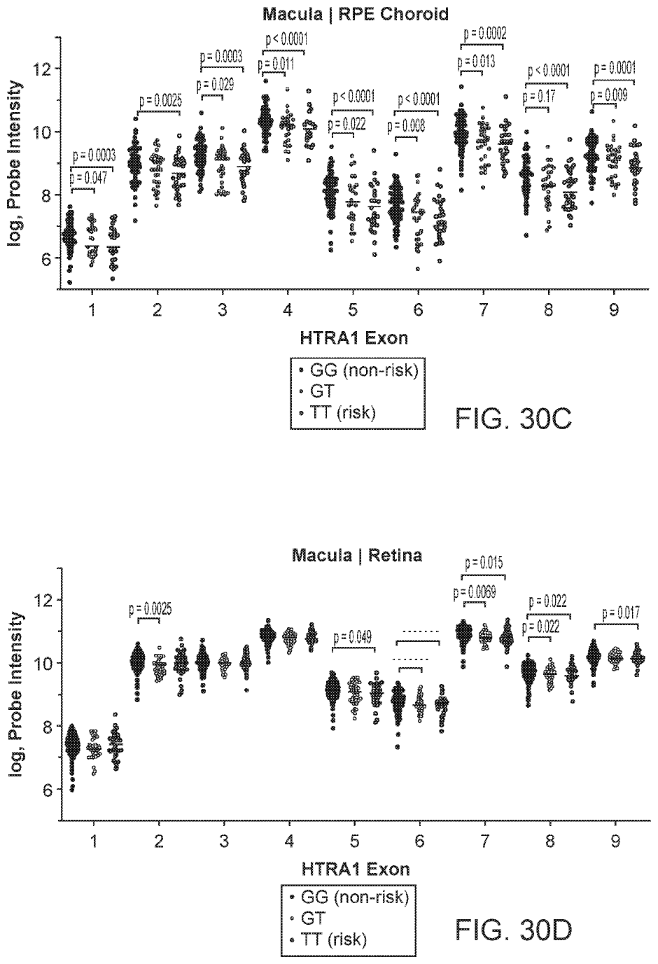

Microarray-based gene expression analyses were conducted using two independent sets of human donor eye tissues (comprising 80 and 200 donor samples, respectively). The results showed that HTRA1 mRNA levels were decreased in RPE-choroid tissue of human donors homozygous or heterozygous for a risk allele at rs10490924, relative to non-risk donors homozygous for the non-risk allele (FIG. 2 and FIG. 30). The results of reduced HTRA1 mRNA levels in RPE-choroid tissue were confirmed by qRT-PCR analyses (FIGS. 3A and 3B). Allele-specific expression analysis using mRNA from heterozygous donor RPE tissue samples also showed a significant and reproducible decrease of risk allele mRNA relative to wild-type allele mRNA. The number of copies of mRNA with the non-risk allele versus the risk allele was determined using digital PCR and a Taqman SNP assay. There were no allele specific mRNA changes detected in the choroids or retinas from the same donor samples (FIG. 4). These results, obtained from an allele-specific HTRA1 expression assay, strongly support that the AMD-associated risk allele results in reduced expression of HTRA1 mRNA.

The results also show that the reduction of HTRA1 mRNA is tissue specific—it was only detected in RPE, but not in retina. FIG. 2, FIG. 3A, FIG. 3B and FIG. 30 show that the HTRA1 mRNA levels decreased in RPE-choroid tissue of patients having one or two risk alleles at rs10490924, relative to non-risk donors with two, wild-type, non-risk alleles, but the HTRA1 expression in retina remained unchanged.

14.2 HTRA1 Expression is Enriched in Bruch's Membrane and Choroid in Eye Tissues from Human Non-AMD Samples

This example shows that HTRA1 mRNA is secreted in a polarized manner and enriched in Bruch's membrane and choroid in human eye tissues from non-AMD samples.

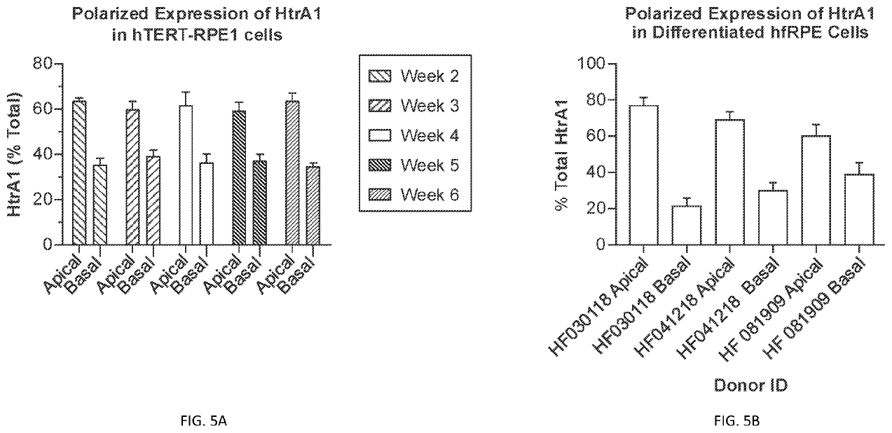

In both polarized hTERT-RPE1 cells (a human RPE cell line, see Bodnar et al., 1998, Science 279:349-52) and human fetal RPE cells, HTRA1 protein were secreted in a polarized manner with 70% of HtrA1 secreted apically and 30% secreted basally (FIGS. 5A and 5B). Basal secretion of HTRA1 may be the exclusive source of sub-RPE HTRA1 in the elderly, as the HTRA1 trimer is not expected to gain access to the sub-RPE space from the choriocapillaris, due to selective permeability of Bruch's membrane and RPE basal lamina (Moore, D J et. al., 2001). The interface between the RPE and Bruch's membrane is highly enriched for extracellular matrix (ECM) proteins and it is likely that HTRA1, a secreted serine protease with demonstrated abilities to degrade various ECM proteins, has an age-related essential function in this space. Relative to retina, RPE and choroid, the data indicate that HTRA1 protein were enriched in extracts derived from Bruch's membrane with or without in samples from patients who do not have AMD (FIG. 6). Without intending to be bound by a particular theory or mechanism, these results are consistent with a model in which homozygous risk patients, with decreased HTRA1 protein in the sub-RPE space, have altered processing, maintenance or turnover of ECM proteins that results in accumulation of damaged, misfolded and/or aggregated proteins. This may disrupt attachment of RPE tissue to its basal lamina and/or to Bruch's membrane, resulting in a loss of the blood-retina barrier. In support of this, we have demonstrated that both chroidal fibrosis and basal laminar deposits (BLDs)—abnormal extracellular material lying between the RPE plasma membrane and the basal lamina and shown to strongly associate with late-stage AMD—occur in donors homozygous for chromosome 10 risk (Table 1, above). Mutations in several ECM structural proteins that result in their mis-regulation lead to AMD-like diseases including L-ORD (C1qTNF5), Sorsby Fundus Dystrophy (TIMP3), Ehlers Danlos Syndrome type VI (PLOD1) or Doyne's Honeycomb Retinal Dystrophy (EFEMP1). See Hayward et al., Hum. Mol. Genet. 12:2657-67 (2003); Weber et al., Nat. Genet 8 (4):352-6; and Marmorstein et al., PNAS. 99 (20): 13067-72. This suggests that alterations of the ECM and the formation of BLDs likely share a common etiology.

14.3 HtrA1 Protein in Human Ocular Tissues

The goal of an AAV-HTRA1 therapy is to deliver a therapeutic amount of HtrA1 to the eye in order to restore function. To that end, an understanding of HtrA1 concentrations in human and AGM ocular tissues is necessary. We measured the concentrations of HtrA1 by ELISA assay from extracts of various ocular tissues. The concentration in human ocular tissues is summarized in Table 6.

TABLE 6
Concentration of HtrA1 protein in human serum and ocular tissues
HTRA1 Protein (ng/mg total protein)

| Tissue | # samples (n) | Ave | | SD | % CV |
|---|---|---|---|---|---|
| Serum (ELISA) | 249 | 34.1 | ng/ml | 11.0 | 32% |
| Serum (mass spec) | 3 | 36 | ng/ml | | |

| | | | | |
|---|---|---|---|---|
| Retina-XMac | 40 | 4.4 | 1.7 | 39% |
| RPE-XMac | 38 | 7.6 | 3.9 | 52% |
| RPE-Mac | 41 | 9.2 | 14.0 | 151% |
| BM-XMac | 27 | 39.2 | 21.5 | 55% |
| BM/Ch-XMac | 40 | 12.0 | 7.0 | 59% |
| BM/Ch-Mac | 39 | 40.7 | 22.3 | 55% |
| RPE-choroid | 74 | 19.0 | 16.7 | 88% |

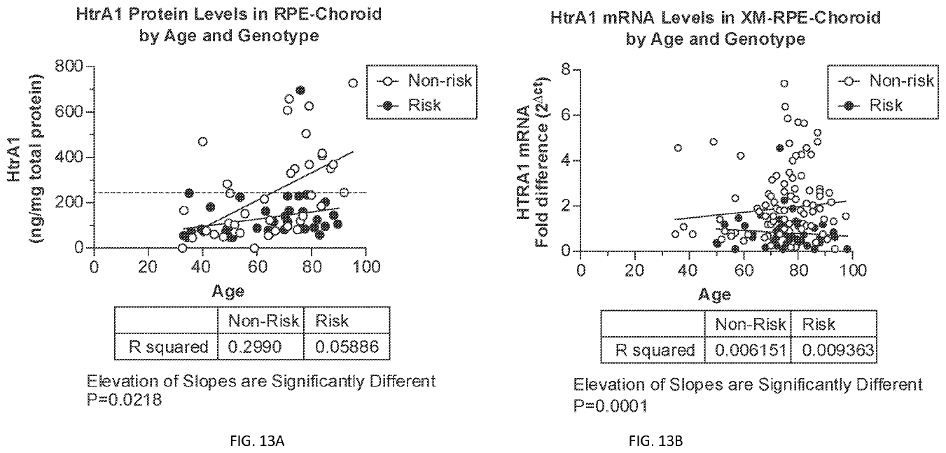

A comparison of HtrA1 protein and mRNA levels in extramacular retina and RPE-choroid as a function of age in donors with or without risk at the Chr10 locus demonstrated that HtrA1 levels are relatively unchanged with age in retina independent of Chr10 risk status (FIG. 12) but significantly increases with age in the RPE-choroid of donors without Chr10 risk (FIG. 13A). In contrast, donors with Chr10 risk show a significant impairment in the age-dependent increase in HtrA1 in RPE-choroid of non-risk donors. This is consistent with a similar age-dependent increase in HTRA1 mRNA in non-risk donors but not in donors with risk (FIG. 13B). Without intending to be bound by a particular theory or mechanism, these results are consistent with a model in which there is an increased demand for HtrA1 with age that is met by transcriptional upregulation of HTRA1 mRNA, leading to translation of more HtrA1 protein but that in donors homozygous for risk, there is a failure to upregulate HTRA1 at either the mRNA or protein levels.

14.4 A 4 Kb Region that Contains Regulatory Elements that Result in Decreased HTRA1 mRNA Expression is Also Associated with AMD Risk

This example shows that the genomic region responsible for causing decreased HTRA1 matches the region that is associated with the risk of developing AMD.

To narrow the region on chromosome 10 that is responsible for AMD (risk region), the HTRA1 allele-specific expression assay was performed using mRNA derived from donors with rare recombination events within the AMD-associated ARMS2/HTRA1 LD block. Donors were heterozygous at the rs1049331 SNP in HTRA1 exon 1, but homozygous for either the risk or non-risk alleles in a portion of the upstream SNPs within the LD block. By using these donors in the allele-specific expression assay and mapping the recombination sites in each donor via DNA sequencing, the HTRA1 promoter SNP and ARMS2 insertion-deletion (indel) region were excluded as causal drivers of decreased HTRA1 mRNA expression. Instead, the region associated with decreased HTRA1 mRNA was mapped to an upstream regulatory region between and including rs11200632 and rs3750846 SNPs that includes rs10490924 (ARMS2 A69S) (Table 8). The same genetic region was found to be associated with elevated risk of AMD disease by Grassmann et al (Genetics 2017) in their analysis of recombinant haplotypes in case/control studies. The finding that the region associated with allele-specific expression of HTRA1 matches the same region associated with AMD risk strongly implies that risk-associated reduction in HTRA1 expression leads to an increase in AMD incidence. This region is designated as the 4 kb AMD risk region.

Table 7, below, shows nine haplotypes of the ARMS2 region, each of which accounts for greater than 2% of the population. Haplotypes H1, H2, H3 and H6 all showed increased Chr 10 AMD risk and all comprise a T allele at SNP rs10490924. This indicates that the presence of the risk allele at rs10490924 (A69S) is associated with development of Chr 10 AMD.

TABLE 7
Haplotypes Within the ARMS2 Region

| | rs12571218 | rs4565845 | rs4751890 | rs10082476 | rs10088714 [symbol] | rs11200616 | rs11200617 | rs10887149 |
|---|---|---|---|---|---|---|---|---|
| H1 | A | C | T | A | C | A | C | G |
| H2 | A | A | T | A | C | A | C | G |
| H3 | A | A | T | A | C | A | C | G |
| H4 | A | A | C | A | C | A | C | A |
| H5 | A | A | C | G | A | A | C | A |
| H6 | A | A | T | A | C | A | C | G |
| H7 | A | A | C | G | A | A | C | A |
| H8 | A | A | C | G | A | A | C | A |
| H9 | A | A | T | A | C | A | C | G |

| | rs11200621 | rs10510110 | rs4752695 | rs2223089 | rs2736911 | rs10490924 | rs17623531 | rs2014307 |
|---|---|---|---|---|---|---|---|---|
| H1 | A | C | G | G | C | T | C | G |
| H2 | G | C | G | G | C | T | C | G |
| H3 | A | C | G | G | C | T | C | G |
| H4 | A | T | G | G | T | G | C | G |
| H5 | A | T | G | G | C | G | C | T |
| H6 | A | C | G | G | C | T | C | G |
| H7 | A | T | C | G | C | G | C | T |
| H8 | A | T | G | G | C | G | C | T |
| H9 | A | T | C | G | C | G | T | G |

| | rs11200638 | rs2672598 | rs12571363 | rs12259258 | rs2736914 | rs2672591 | rs4752699 | rs2672590 |
|---|---|---|---|---|---|---|---|---|
| H1 | A | C | C | A | G | T | G | A |
| H2 | A | C | C | A | G | T | G | A |
| H3 | A | C | C | A | G | T | G | A |
| H4 | G | C | C | A | C | T | G | C |
| H5 | G | C | C | A | G | A | G | A |
| H6 | A | C | C | A | G | T | G | A |
| H7 | G | T | C | A | G | A | G | A |
| H8 | G | T | C | A | G | A | G | A |
| H9 | G | T | C | A | G | A | A | A |

| | rs7093894 | rs2672588 | rs2672587 | rs4237540 | rs2736917 | rs2284668 | rs2253755 |
|---|---|---|---|---|---|---|---|
| H1 | C | T | G | A | A | T | G |
| H2 | C | T | G | A | A | T | G |
| H3 | C | T | G | A | A | T | G |
| H4 | C | C | C | A | G | T | A |
| H5 | C | C | C | G | A | T | A |
| H6 | C | T | G | G | A | T | A |
| H7 | C | T | C | A | A | C | A |
| H8 | C | T | C | G | A | T | A |
| H9 | A | T | C | G | A | T | A |

| | | | | | | | | | Case, | | |
|---|---|---|---|---|---|---|---|---|---|---|---|
| | | | | rs2736923 | | | | | Control | Chi | |
| | rs2300431 | rs2268346 | rs736962 | rs11200655 | rs876790 | rs2250804 | rs2268356 | Freq. | Freqs. | Square | P Value |
| H1 | G | G | A | A | CT | C | C | 0.109 | 0.0132, 0.054 | 11.086 | 9.00E−04 |
| H2 | G | G | A | A | CT | C | C | 0.068 | 0.082, 0.038 | 5.346 | 0.0208 |
| H3 | G | G | A | A | CT | C | C | 0.056 | 0.067, 0.032 | 3.967 | 0.0464 |
| H4 | G | G | A | A | CT | T | T | 0.049 | 0.050, 0.074 | 2.209 | 0.1372 |
| H5 | A | A | A | A | CC | T | C | 0.031 | 0.028, 0.061 | 6.321 | 0.0119 |
| H6 | G | G | A | A | CT | T | T | 0.029 | 0.038, 0.004 | 6.79 | 0.0092 |
| H7 | A | G | A | A | CT | T | T | 0.029 | 0.026, 0.061 | 7.798 | 0.0052 |
| H8 | G | G | A | A | CT | T | T | 0.026 | 0.027, 0.040 | 1.114 | 0.2911 |
| H9 | G | G | A | A | CT | C | C | 0.023 | 0.024, 0.030 | 0.271 | 0.6028 |

[symbol]

indicates data missing or illegible when filed

TABLE 8
Analysis of allele-specific expression of HTRA1 in RPE−choroid of human donors with genetic
recombination within the 10q26 GWAS LD block associated with AMD
Causal Region

| SNP: | rs61871744 | rs11200630 | rs61871745 | rs11200632 | rs11200633 | rs61871746 | rs61871747 |
|---|---|---|---|---|---|---|---|
| non-risk | T | T | G | A | C | T | C |
| risk | C | C | A | G | T | C | T |
| Het Donor | 1 | 1 | 1 | 1 | 1 | 1 | 1 |
| Recombinant | 2 | 2 | 2 | 2 | 2 | 2 | 2 |
| Donors | 2 | 2 | 2 | 2 | 2 | 2 | 2 |
| | 0 | 0 | 0 | 0 | 0 | 0 | 0 |
| | 0 | 0 | 0 | 0 | 0 | 0 | 0 |
| | 0 | 0 | 0 | 1 | 1 | 1 | 0 |
| | 0 | 0 | 1 | 1 | 1 | 1 | 1 |

| SNP: | rs10490924 | 10:124214600 | rs36212731 | rs36212732 | rs36212733 | rs3750848 | rs3750847 |
|---|---|---|---|---|---|---|---|
| non-risk | G | G | G | A | T | T | C |
| risk | T | GGT | T | G | C | G | T |
| Het Donor | 1 | 1 | 1 | 1 | 1 | 1 | 1 |
| Recombinant | 2 | 2 | 2 | 2 | 2 | 2 | 2 |
| Donors | 2 | 2 | 2 | 2 | 2 | 2 | 2 |
| | 0 | 0 | 0 | 0 | 0 | 0 | 0 |
| | 0 | 0 | 0 | 0 | 0 | 0 | 0 |
| | 1 | 1 | 1 | 1 | 1 | 1 | 1 |
| | 1 | 1 | 1 | 1 | 1 | 1 | 1 |

| | | | | | | | % Risk |
|---|---|---|---|---|---|---|---|
| | | INDEL | | | | | allele in |
| SNP: | rs3750846 | [symbol] | rs3793917 | rs11200638 | rs1049331 | ASE | RPE |
| non-risk | T | 443 bp | C | G | C | | |
| risk | C | 54 bp | T | A | T | | |
| Het Donor | 1 | 1 | 1 | 1 | 1 | Y | 41.0 |
| Recombinant | 2 | 2 | 2 | 1 | 1 | N | 52.3 |
| Donors | 2 | 2 | | 1 | 1 | N | 49.4 |
| | 0 | 1 | 1 | 1 | 1 | N | 50.0 |
| | 1 | 1 | | 1 | 1 | M | 44.4* |
| | 1 | 1 | 1 | 1 | 1 | Y | 43.5 |
| | 1 | 1 | 1 | 1 | 1 | Y | 41.5 |

*Ave (n = 3) [symbol]
indicates data missing or illegible when filed

14.5 Inverse Relationship Between HTRA1 and IncSCTM1 mRNA Expression

This example demonstrates the inverse relationship between HTRA1 and incSCTM1 allele-specific mRNA expression. A long non-coding RNA (LOC105378525) was identified and mapped to a region that overlaps with the 4 kb AMD risk region. Our analysis showed that there are four predicted variants of the IncRNA that include XR_946382, XR_946383, XR_946384 and XR_946385 (FIG. 7). Various isoforms of IncSCTM1 that have been detected in hTERT-RPE1 cells, retina and RPE tissue are shown in Table 9. This IncRNA was transcribed in the anti-sense orientation from the HTRA1 promoter and it appears that HTRA1 and LOC105378525 may share an overlapping divergent promoter. We have designated this IncRNA as IncSCTM1. RACE (rapid amplification of cDNA ends) PCR analysis of RNA from retina and RPE indicates that exon 3, as mapped in XR_946382 variant 1, extends approximately 1 kb further than reported. This is supported by RNA-seq data from the GTEx Consortium in which reads are mapped to this extended region in various tissues. RACE PCR also indicates that the alternate exon 3, as mapped in variant 4 (XR_946385), begins at either hg38 Chr 10:122,455, 021 (alt ex3a) or Chr 10:122,454, 857 (Alt ex3b) and extends in the anti-sense direction to Chr 10:122,454, 457. Sequence analysis indicates that the alt ex3a variant contains the rs10490924 SNP, the risk form of which is associated with elevated AMD risk, whereas alt ex3b does not. Furthermore, variants comprising exon 3 instead of alternative exon 3, do not contain rs10490924 or any other SNPs in the 4 kb AMD risk region The IncSCTM1 alternative exon 3 almost completely overlaps with ARMS2 exon 1.

TABLE 9
EXPRESSION OF IncSCTM1 ISOFORMS

| Category | Variant ID | hTERT-RPE1* | Retina | RPE-choroid |
|---|---|---|---|---|
| Long Isoforms | IncSTM-L1 | ♦ | ♦ | ♦ |
| (Exon 3) | IncSTM-L2 | | ♦ | ♦ |
| | IncSTM-L3 | | ♦ | ♦ |
| | IncSTM-L4 | ♦ | ♦ | ♦ |
| | IncSTM-L5 | | | ♦ |
| | IncSTM-L6 | | ♦ | |
| Short Isoforms | IncSTM-S1 | ♦ | | ♦ |
| (Alt Ex3) | IncSTM-S2 | | | ♦ |
| | IncSTM-S3 | | ♦ | |

*Not fully screened so other variants may exist in these cells

Because IncRNAs sometimes regulate expression of nearby genes, the relationship between levels of HTRA1 mRNA and IncSCTM1 ncRNAs were examined using allele-specific expression assays for HTRA1 (rs1049331, exon 1) and IncSCTM1 (rs11200638, exon 1). mRNA from heterozygous donors was used in the assay and any deviation from the expected 50:50 ratio was indicative of allele-specific expression. Gene-specific primers were used for cDNA synthesis to ensure that transcripts come from the expected strand. It was observed that the allele-specific expression of HTRA1 inversely correlated with the allele-specific expression of IncSCTM1. The results also showed that mRNA derived from the risk allele of IncSCTM1 was expressed at higher levels than that derived from the non-risk allele, whereas mRNA derived from the risk allele of HTRA1 was decreased relative to that of the non-risk allele (FIG. 8). The risk allele analyzed in FIG. 8 was at rs11200638, which is in the LD block associated with AMD that includes rs10490924. rs11200638 is also within exon 1 of IncSCTM1 and present in all isoforms of IncSCTM1. RNA-seq analyses of retina and RPE indicate that HTRA1 mRNA was expressed at >400-fold higher levels than IncSCTM1 mRNA (data not shown). If IncSCTM1 regulates HTRA1 expression, it seems unlikely that it could regulate HTRA1 in trans and instead, most likely IncSCTM1 would regulate expression of HTRA1 via cis effects on HTRA1 transcription.

14.6 A 2 kb AMD Risk Region that Contains Regulatory Elements that Result in Decreased HTRA1 mRNA Expression

Epigenetic Markers

This example describes a newly identified 2 kb region that is responsible for regulating HTRA1 transcription and transcriptional activators associated therewith. Examination of publicly available epigenetic data (ATAC-Seq and DNase-Seq) derived from human fetal RPE tissue or from induced pluripotent stem cells differentiated into RPE (iPSC-RPE) indicated approximately 2.0 kb region of open chromatin that overlaps the 4 kb AMD risk region. Furthermore, this same 2 kb region comprises epigenetic markers of active transcription, including H3K27 acetylation. This 2 kb region overlaps both ARMS2 exon 1-intron 1 and the alternative exon 3 of IncSCTM1 (FIG. 10) as well as other SNPs in the 4 kb AMD risk region, including rs36212731, rs36212732, rs36212733 and rs3750848.

ChIP-Seq analysis of retina and RPE extracts was performed with antibodies targeting histone marks associated with enhancer elements, including monomethylated histone H3 lysine 4 (H3K4me1) and acetylated histone H3 lysine 27 (H3K27Ac). In RPE extracts, both marks were present in a peak that overlaps the open chromatin region while only the H3K4me1 mark was present in retina. These results support the hypothesis that this approximately 2 kb region within the ARMS2 intron functions as a tissue-specific enhancer element.

SNPs Located in the 2 kb Region

Eight SNPs (rs10490924, rs144224550, rs36212731, rs36212732, rs36212733, rs3750848, rs3750847, rs3750846) are within the Chr10 LD block.

14.7 The 2 Kb Region Includes a LHX2 Consensus Binding Motif Present in the Wild-Type but not the Risk Allele

We scanned the sequence for known transcription factor binding motifs using HOMER, JASPER and TRANSFAC tools, comparing the non-risk and risk genotypes for each AMD-associated SNP. This analysis identified the rs36212733 variant in the intron of ARMS2 as a transcription factor binding site with predicted genotype-dependent differential binding of LHX2, POU6F1 and/or ZNF33. Of these, LHX2 mRNA expression is modest (FPKM>20) in RPE whereas POU6F1 and ZNF333 expression is low (FPKM<2). Of those screened, only LHX2 had a consensus binding motif located within the wild-type but not risk sequence, and that there was a near perfect match for this sequence motif (FIG. 9) in the intron of ARMS2 and IncSCTM1. LHX2 is also expressed in some cell types in retina and choroid and thus, is not an RPE-specific transcription factor. The rs36212733 SNP associated with risk of AMD disrupts one of the critical residues within this motif by converting the ‘T’ at position 6 to a ‘C’. The LHX2 site is surrounded by two potential binding sites for members of the SoxE family of transcription factors but these do not overlap the Chr10-associated SNPs.

Without intending to be bound by a particular theory or mechanism, these data are consistent with a model in which LHX2 binds to a motif that overlaps rs36212733 in the 4 kb AMD risk region that lies upstream of HTRA1 and overlaps both the ARMS2 and IncSCTM1 genes. In non-risk RPE cells, LHX2 binds this motif along with an RPE-specific co-factor and acts as an enhancer of HTRA1 transcription, thereby increasing the expression of HTRA1 above the basal level in an RPE-specific manner. It is unknown what the RPE-specific co-factor is at this time and whether its expression is affected by age-related factors or other external signals. If binding of LHX2 to this enhancer impedes the ability of the RNA polymerase to proceed with transcription of IncSCTM1, this would lead to a decrease in expression of the IncSCTM1 gene. Since LHX2 is predicted to only bind the non-risk allele, this leads to an allelic imbalance with reduced levels of the non-risk allele relative to the risk allele in heterozygous donors. This hypothesis is supported by our finding that there is an inverse relationship between the allele-specific expression of HTRA1 and IncSCTM1 in RPE cells (FIG. 8). Finally, in a non-RPE cell, binding of LHX2 to the non-risk sequence would not enhance HTRA1 expression due to the absence of the RPE-specific co-factor. However, it would still interfere with IncSCTM1 transcription, resulting in lower levels of expression of the non-risk allele. Thus, in non-RPE tissue, there is no allele-specific expression of HTRA1 but allele-specific expression of IncSCTM1 remains, as long as the cells express LHX2.

14.8 LHX2 Binds More Strongly to the Wild-Type Probe than to Probe Containing the Risk Sequence

To test whether LHX2 can bind this sequence of DNA, electrophoretic mobility shift assay (EMSA) were performed using nuclear extracts from HEK293 cells transfected with plasmid encoding LHX2 or empty vector and with biotinylated oligo probes containing the non-risk (WT) sequence or the risk sequence (RISK) of the Chr10 region encompassing rs36212732 and rs36212733. Probes containing the scrambled sequence of this region (SCR, negative control) or a previously reported LHX2 binding site (POS, positive control) (Muralidharan et al. (2017)J. Neurosci 37(46):11245-54) were also used. The results show that LHX2 bound to both the wild-type and risk probes as well as the positive control probe, but not to the scrambled probe (FIG. 10A). Addition of an anti-LHX2 antibody to the reaction caused a super shift of the LHX2 band, confirming the identity of LHX2, and addition of unlabeled probe resulted in the loss of the band (FIG. 10B). Finally, we compared the binding affinity of LHX2 for the wild-type versus risk probes by adding increasing amounts of the probes into the reactions. LHX2 bound more strongly to the wild-type probe than to probe containing the risk sequence (FIG. 11) Overall, these results show that LHX2 can bind to the sequence encompassing rs36212733 and that the presence of the risk allele significantly decreases binding of LHX2 to this sequence. In combination with the epigenetic data described above and the eQTL association of rs36212733 with HTRA1 expression, our results support the hypothesis that this region functions as an enhancer for HTRA1 expression in RPE cells and that LHX2 may contribute to HTRA1 expression.

14.9 Use of a CRISPRa System to Upregulate HTRA1 mRNA Expression (Lentiviral Plasmids)

Microarray and qRT-PCR analysis of donor RPE tissue has demonstrated that the level of HTRA1 mRNA was downregulated by approximately 30% in donors with homozygous risk at Chr10 compared to donors with no risk. The SAM CRISPRa system was selected to restore the expression of HTRA1 in RPE tissue to levels similar to non-risk donors while minimizing any off-target effects on gene expression. The SAM system consists of two plasmids: LentiSAM plasmid (SAM; Addgene #92062) encoding both the sgRNA and the dCas9-VP64 fusion protein, and LentiMPH (MPH; Addgene #92065) plasmid encoding a fusion protein comprised of the MS2 affinity tag and the p65 and HSF1 transcription factors.

Using the Benchling software, sgRNAs were designed to cover the promoter region of HTRA1 (FIG. 14). These sequences, along with a published sgRNA sequence targeting IL1B, were cloned into BsmBI sites adjacent to the U6 promoter within the lentiSAM v2 (Puro) plasmid (Addgene). Cells were transfected with the sgRNA-LentiSAMv2 (Puro) plasmid with or without equimolar amounts of the Addgene (Neo) plasmid (5 μg total) by electroporation. After 24-96 hours, total RNA was extracted and purified using an RNeasy kit (Qiagen, Cat. #74106). Complementary DNA was generated using 1 μg of total RNA and the SuperScript IV VILO Master Mix kit (Invitrogen, Cat. #11756050). Quantitative PCR was performed using 50 ng of cDNA and Tag Man gene expression assays (Applied Biosystems) for HTRA1 (Hs01016151_m1), IL1B (Hs01555410_m1), and GAPDH (hs03929097_g1) following the standard protocol.

We tested the ability of the sgRNAs listed in TABLE 10 to upregulate HTRA1 expression using the SAM CRISPRa system (Konerman et al., Nature, 517(7536) 2015).

TABLE 10
Guide Sequences For Crispr Activation

| | | | | | | Off- |
|---|---|---|---|---|---|---|
| | | | | | On- | tar- |
| Name | SEQ | Strand | Sequence | PAM | target | get |
| sgRNA-P1 | 15 | − | CACGCGGGAC | GGG | 64.0 | 89.3 |
| | | | CCTGACCGCG | | | |
| sgRNA-P2 | 16 | − | TGCCCCCGGT | CGG | 53.6 | 82.9 |
| | | | ACCCCGCACG | | | |
| sgRNA-P3 | 17 | − | CCCGGGGCGT | CGG | 61.9 | 85.1 |
| | | | TGCGGCACCG | | | |
| sgRNA-P4 | 18 | − | CCTGCAAATC | GGG | 61.7 | 90.9 |
| | | | GCCGGACTGG | | | |
| sgRNA-P5 | 19 | + | CTGCGGAAAG | GGG | 66.2 | 82.8 |
| | | | CGAATATGTG | | | |
| sgRNA-P6 | 20 | + | GGAAACTGAG | GGG | 64.1 | 85.5 |
| | | | TCCCGCGAGA | | | |
| sgRNA-P7 | 21 | − | GGGCTGGCCG | GGG | 51.9 | 60.3 |
| | | | GATGGCGGGA | | | |
| sgRNA-P8 | 22 | − | AGCGGTGACG | GGG | 63.2 | 93.5 |
| | | | AGCCGCCTTG | | | |
| sgRNA-P9 | 23 | + | ACTGAGTCCC | CGG | 51 | 39 |
| | | | GCGAGAGGGC | | | |
| sgRNA-P10 | 24 | − | CGCACAGGCC | CGG | 46 | 42 |
| | | | GGCCCTCTCG | | | |
| sgRNA-P11 | 25 | − | GCGCGGGCGG | AGG | 26 | 36 |
| | | | GGCAGCGCAC | | | |
| sgRNA-P12 | 26 | + | CCCGCCAGCA | CGG | 19 | 37 |
| | | | CCGCCGTGCC | | | |
| sgRNA-P13 | 27 | − | AGGGCGGGCG | CGG | 25 | 30 |
| | | | CGCCCGGGCA | | | |
| sgRNA-P14 | 28 | + | CGCCCTGCCC | CGG | 51 | 37 |
| | | | CCTCCGCGGG | | | |
| sgRNA-P15 | 29 | − | GGACCGGGAC | GGG | 35 | 45 |
| | | | CGCCCGCGGA | | | |
| sgRNA-P16 | 30 | − | GGAGGGCGGG | GGG | 39 | 31 |
| | | | CGGCTGGACC | | | |
| sgRNA-P17 | 31 | − | GGGCACGGCG | GGG | 27 | 37 |
| | | | CCCGGGATGG | | | |
| sgRNA-P18 | 32 | + | CGCCGTGCCC | CGG | 58 | 34 |
| | | | GTCCCCAAGG | | | |
| sgRNA-P19 | 33 | + | GTCACCGCTG | GGG | 40 | 46 |
| | | | CGAGGCCAAT | | | |

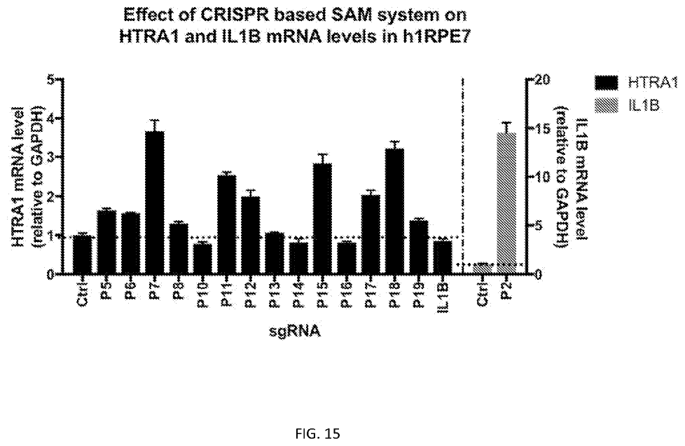

FIG. 15 shows that most of the sgRNAs upregulated HTRA1 to some level. P7 and P18 sgRNAs showed the highest increase in HTRA1 levels (an approximately 3.6- and 3.2-fold increase for P7 and P18 sgRNA, respectively).

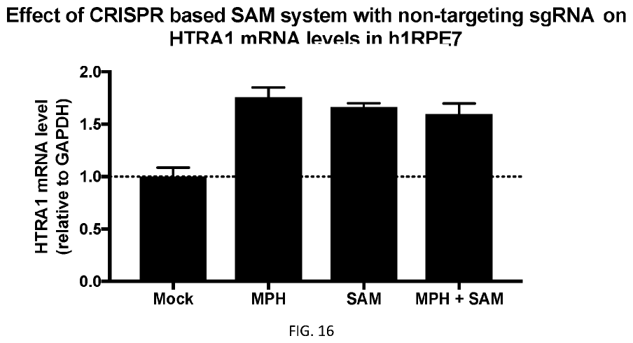

To evaluate whether either of the plasmids had off-target effects on mRNA levels, we transfected h1RPE7 cells with the SAM components individually (MPH or SAM) or together (MPH+SAM). In these experiments, the sgRNA in the SAM plasmid does not target any known human genes (Ctrl sgRNA). As shown in FIG. 16, relative to mock-transfected cells, the MPH plasmid increased the level of HTRA1 by approximately 1.8-fold in the absence of an HTRA1-targeting sgRNA. Similarly, there was an approximately 1.6-fold increase in HTRA1 in cells transfected with the SAM plasmid alone and this was not increased further by the presence of the MPH transcription factors. These data indicate that the SAM system components may promote a general increase in transcription.

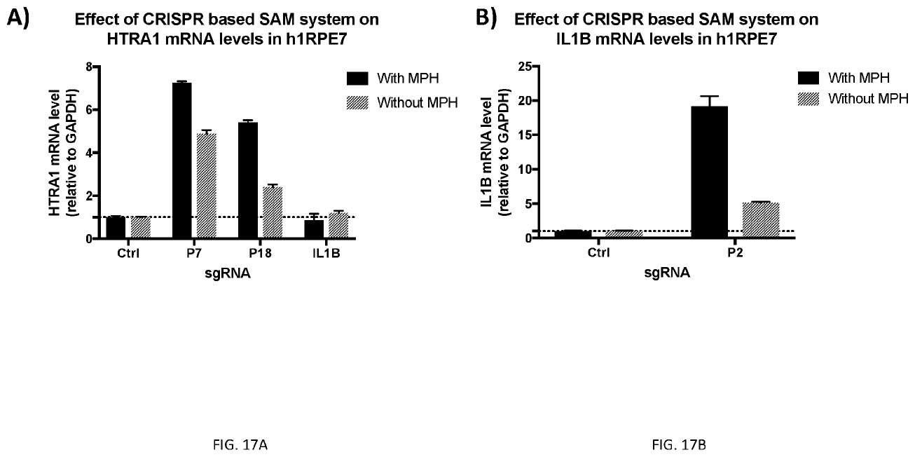

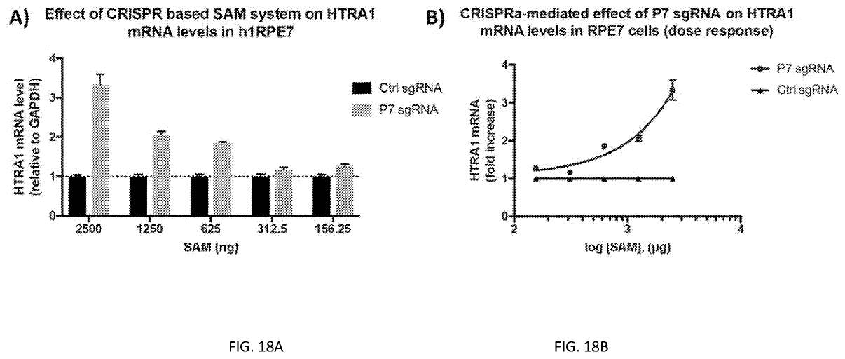

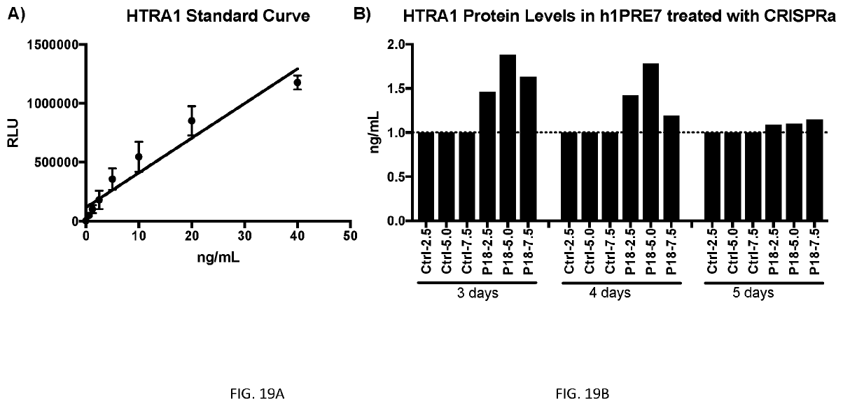

Next, we performed experiments to determine whether the MPH plasmid was required for the upregulation of HTRA1. We tested our strongest HTRA1-targeting sgRNAs (P7 and P18, FIG. 17) in the absence or presence of the MPH plasmid for their effects on HTRA1 expression in h1RPE7 cells. We found that even in the absence of the MPH plasmid, HTRA1 levels are increased by the SAM plasmids (5-fold for P7 and 2.5-fold for P18), albeit at reduced levels compared to levels when MPH is included (7-fold for P7 and 5-fold for P18). Thus, it appears that a single plasmid encoding an HTRA1-targeting sgRNA and the dCas9-VP64 transactivator is sufficient to upregulate HTRA1 expression. Additionally, the effects if the SAM system are concentration dependent with HTRA1 levels decreased with decreasing amounts of the SAM plasmid (FIG. 18).

14.10 The Increase in HTRA1 mRNA Correlates with an Increase in HTRA1 Protein Levels

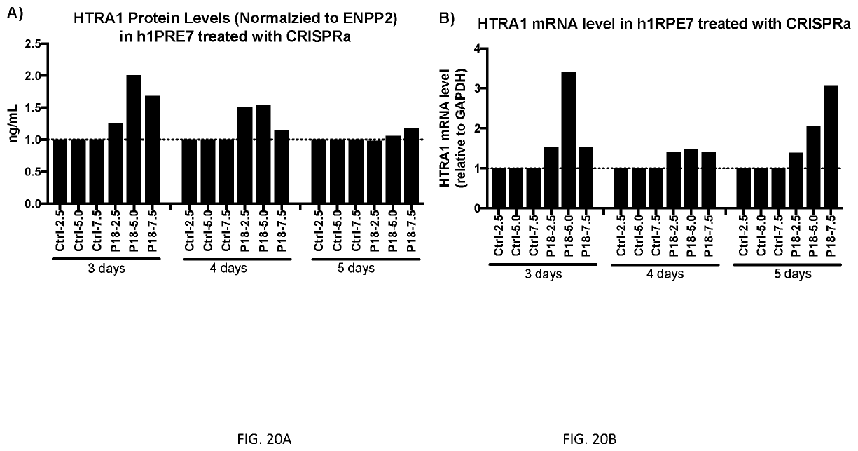

Preliminary experiments were performed to test whether the increase in HTRA1 mRNA described above correlated with an increase in HtrA1 protein levels. Using an ELISA assay, we measured HtrA1 protein concentrations in the cell culture supernatant of h1RPE7 cells transfected with the LentiSAM plasmids. We transfected h1RPE7 cells with different amounts of P18 sgRNA-LentiSAM plasmids (2.5, 5.0 and 7.5 μg) for different time points (3, 4 and 5 days). We also transfected the cells with non-targeting sgRNA (Ctrl) to serve as a control. FIG. 19 shows that HTRA1 protein levels were increased specifically in P18 sgRNA-LentiSAM transfected cells 3 and 4 days after transfection. The protein level reached its maximum with 5 μg of LentiSAM plasmid. FIG. 20 shows HTRA1 protein levels after normalization to ENPP-2 protein. These initial results data indicate that relative to control, HTRA1 protein levels were increased specifically in cells transfected with the P18 sgRNA-LentiSAM plasmid 3 and 4 days post-transfection.

The above data demonstrate that HTRA1 mRNA and protein levels are upregulated in a dose-dependent manner in h1RPE7 cells transiently transfected with P18 sgRNA-LentiSAM plasmid.

14.11 Lentiviral Delivery

The lentiviral particles shown in Table 11 were constructed. These particles comprise the P18-lentiSAM plasmid

TABLE 11
Packaged Lentivirus of sgRNA-LentiSAM plasmids

| Plasmid | Titer (TU/mL) | Amount (μl) | Quantity |
|---|---|---|---|
| Ctrl-LentiSAM | 3.18 × 10\hat{ }8 | 25 | 4 |
| P18-LentiSAM | 2.33 × 10\hat{ }8 | 25 | 4 |
| GFP | 1.49 × 10\hat{ }9 | 50 | 1 |

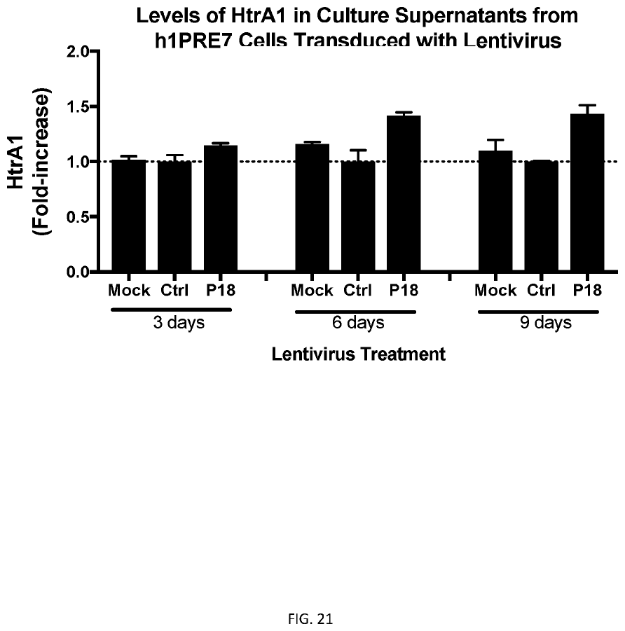

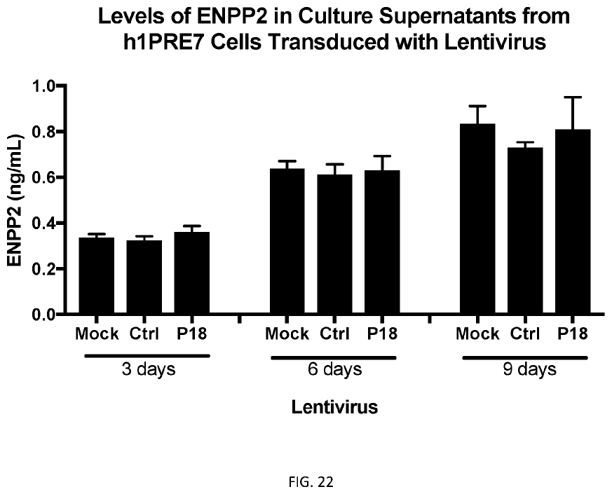

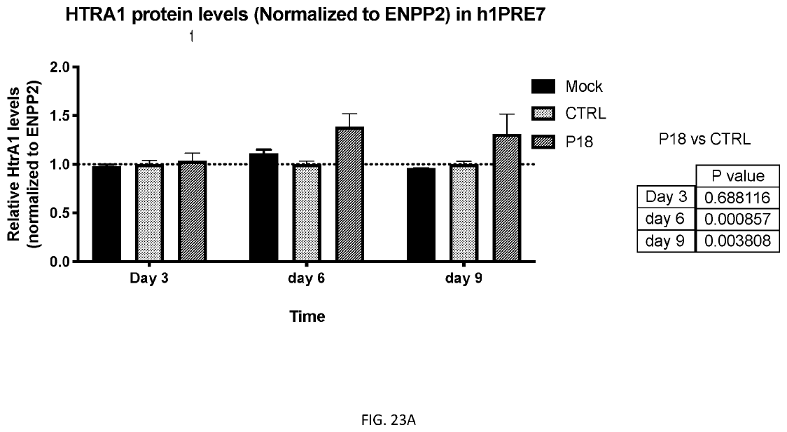

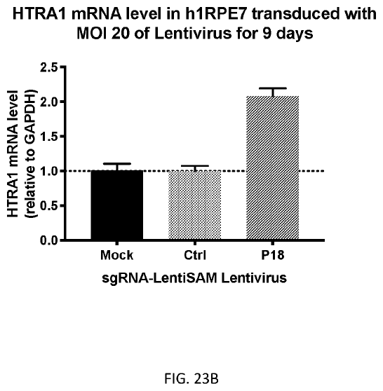

We tested whether the lentiviral delivery of the CRISPR-based SAM system could induce HTRA1 expression in RPE cells. h1RPE7 cells were transduced at an MOI of 20 with lentiviral particles encoding the P18-LentiSAM plasmid and cell culture supernatant was collected every 3 days post-transduction. As a control, cells were transduced with lentiviral particles encoding the non-targeting sgRNA (Ctrl-LentiSAM). Cells were also treated with polybrene-containing media without virus (Mock). HTRA1 protein levels in the cell culture supernatant collected at 3, 6 and 9 days post-transduction were measured by HTRA1 ELISA assay. As shown in FIG. 21, HTRA1 protein levels increased by approximately 40% (1.4-fold increase relative to Ctrl-LentiSAM) in cells transduced with P18-LentiSAM at 6 and 9 days post-transduction compared to cells transduced with Ctrl-LentiSAM viral particles.

To account for possible differences in cell number following transduction of each individual sample, HtrA1 protein levels were normalized to levels of ENPP2 protein, which increased in all samples with time, but was not different between controls and the P18-LentiSAM-treated cells (data not shown). FIG. 23 shows HTRA1 protein levels after normalization to ENPP-2 protein. Levels of HtrA1 still show a ^{˜}30-40% increase following transduction with P18 sgRNA-LentiSAM at 6 and 9 days post-transduction. To test the correlation between HTRA1 mRNA expression and HTRA1 secreted protein levels, mRNA levels of HTRA1 were measured from total RNA extracted at 9 days post-transduction (FIG. 23). HTRA1 mRNA levels were increased approximately 2-fold in cells treated with P18-LentiSAM relative to cells treated with Ctrl-LentiSAM. Thus, both HTRA1 mRNA and protein levels were increased in response to the P18-LentiSAM treatment. This indicates that viral delivery of the CRISPR-based SAM system can successfully induce HTRA1. AAV2-HTRA1 plasmids to upregulate HTRA1

The experiments in this example were conducted to develop an AAV2-based vector carrying the HTRA1 gene under the control of a suitable RPE-specific promoter to restore the wild-type expression level of HTRA1 in the RPE of AMD patients. The HTRA1 gene was subcloned from the pCTM16 plasmid into pCTM295 using KpnI and SphI restriction sites to generate pTR-HTRA1 (pCTM289). Fragments of the BEST1 and RPE65 promoter were PCR amplified using primers with Acc65I (Forward) and BamHI restriction sites (Reverse). The PCR products were digested with Acc65I and BamHI and subcloned into pCTM289 at these sites to generate HTRA1 expression plasmids under the control of BEST1- and RPE65-derived promoter fragments.

Table 12 shows the primers used to prepare each of the promoter fragments from the BEST1-promoter (SEQ ID NO:11) and the RPE65 promoter fragment (SEQ ID NO: 12). RPE1 cells that stably express an HTRA1 3-UTR-targeted shRNA plasmid (and thus, express low levels of endogenous HTRA1) were transfected with 5 μg of indicated plasmids by electroporation using 100 μl Neon tips or by lipid transfection in 96 well plates containing 10,000 cells per well. A total of 0.15 μl of Lipofectamine 3000, 0.2 μl P3000 and 100 ng DNA were used. Cell culture supernatant and RNA were collected 24-96 hours post-transfection. Our standard HTRA1 ELISA was performed to measure levels of protein. Total RNA was extracted from the cells using an RNeasy kit (Qiagen, cat #74106). Complementary DNA was generated using 500 ng of total RNA and SuperScript IV VILO Master Mix kit (Invitrogen, cat #11756050). Quantitative PCR was performed using 50 ng of cDNA and TaqMan gene expression assays (Applied Biosystems) for HTRA1 (Hs01016151_m1) and GAPDH (hs03929097_g1).

TABLE 12
Different Truncations of BEST1 and RPE65 Promoters
Cloned Into pTR-HTRA1 plasmid (pCTM289)

| | | | Forward | | Reverse | |
|---|---|---|---|---|---|---|
| Promoter | Plasmids | Size | Primer | Seq | Primer | Seq |
| BEST1 | BEST1_418 | 418 | F22 | 57 | R19x | 58 |
| (VMD2) | BEST1_723 | 723 | F30 | 59 | R28 | 60 |
| | BEST1_340 | 340 | F22 | 61 | R20 | 62 |
| | BEST1_699 | 699 | F22 | 63 | R28 | 64 |
| | BEST1_293 | 293 | F13 | 65 | R20 | 66 |
| | BEST1_295 | 295 | F25 | 67 | R19 | 68 |
| | BEST1_388 | 388 | F6 | 69 | R4 | 70 |
| RPE65 | RPE65_316 | 316 | F28 | 71 | F26 | 72 |
| | RPE65_750 | 750 | F31 | 73 | R9 | 74 |
| | RPE65_518 | 518 | F10 | 75 | R20 | 76 |
| | RPE65_146 | 146 | F7 | 77 | R8 | 78 |
| | RPE65_409 | 409 | F25 | 79 | R8 | 80 |
| | RPE65_370 | 370 | F7 | 81 | R9 | 82 |
| | RPE65_330 | 330 | F26 | 83 | R9 | 84 |
| | RPE65_358 | 358 | F10 | 85 | R8 | 86 |

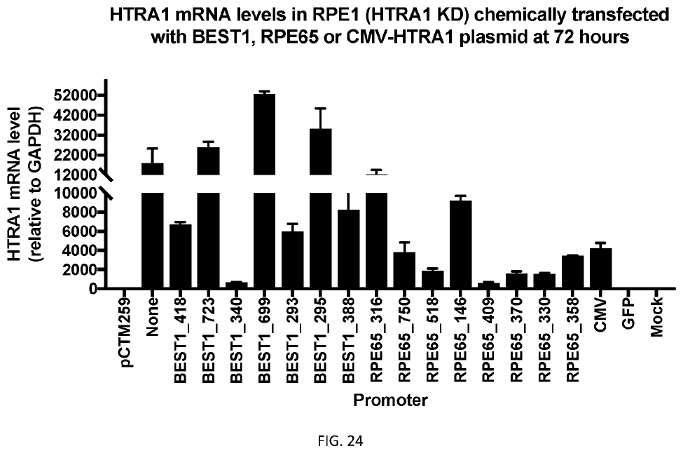

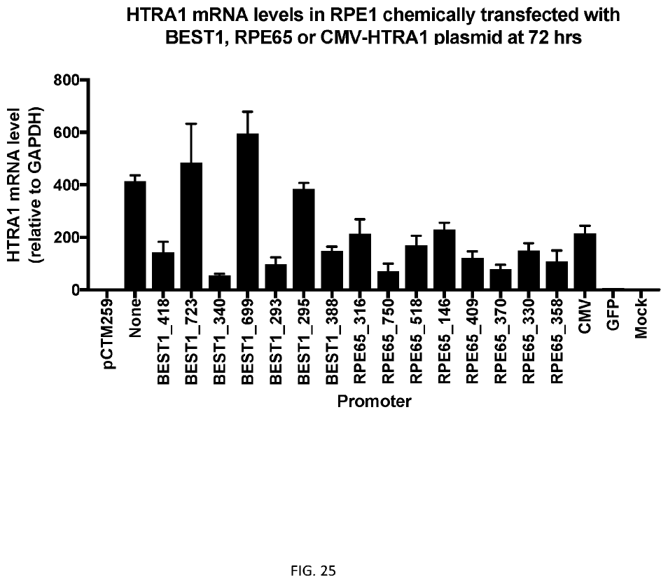

We tested these AAV-HTRA1 plasmids for HTRA1 overexpression in a pooled population of RPE1 clones (7-6 and 7-7) with stable knockdown of HTRA1 (>90%) using electroporation. Because HTRA1 levels are low in these cells, the signal-to-noise ratio is improved and will increase the sensitivity of the assay. As a negative control, we used pCTM259 which encodes for a smCBA-driven CFH gene in the same AAV vector backbone as our plasmids. CFH overexpression does not affect HTRA1 expression (data not shown). FIG. 24 shows that all of the BEST1- and RPE65-driven HTRA1 constructs increased HTRA1 to varying degrees when compared to cells transfected with pCTM259.

We also tested these plasmids in parental RPE1 cells. As shown in FIG. 25, HTRA1 mRNA increased in cells transfected with the BEST1- and RPE65-HTRA1 plasmids compared to the negative control plasmid (pCTM259). The pattern of HTRA1 expression relative to each other is similar in both cell lines.

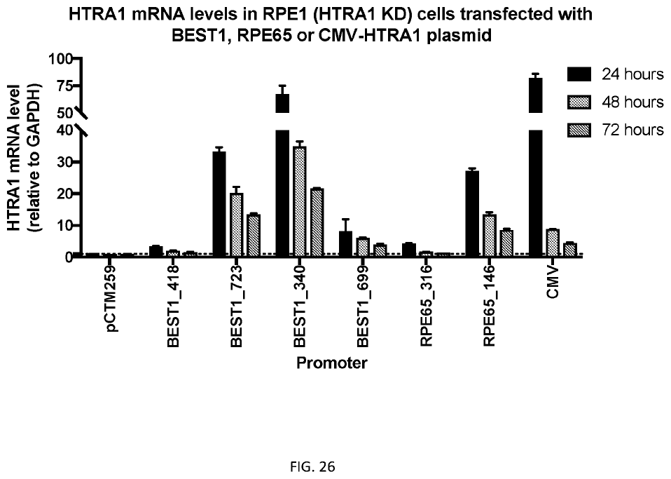

Six constructs, including BEST1_723, BEST_699, BEST1_418, BEST1_340, RPE65_316, and RPE65_146, were selected for further testing. A time course analysis was performed to measure both mRNA and protein levels of HTRA1 in cells transfected with our strongest candidate plasmids. Transfections were performed by electroporation using plasmid DNA prepared with the endotoxin-free maxi-prep kit to reduce toxicity while maximizing the transfection efficiency. Relative to the control plasmid (pCTM259), levels of HTRA1 mRNA are strongly increased by several of the AAV-HTRA1 plasmids driven by the BEST1_723, BEST1_340 and RPE65_146 promoters (FIG. 26). With each of these plasmids, mRNA levels peaked at 24 hours and gradually decrease at the 48- and 72-hour time points. With the positive control CMV-driven promoter, HTRA1 levels also peaked at 24 hours but sharply decrease by the 48- and 72-hour time points.

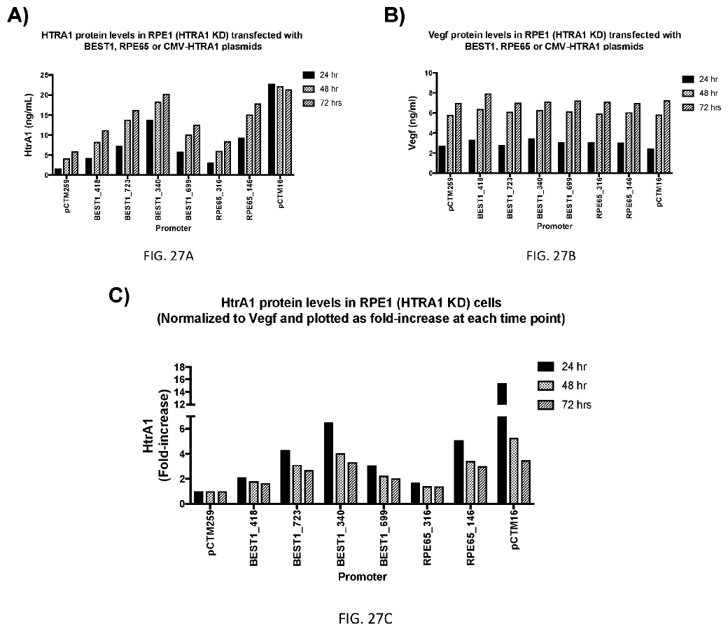

We examined the kinetics of HTRA1 protein expression and normalized it to levels of VEGF, which is not affected by the overexpression of HTRA1 (compare pCTM259 control to each of the other samples in FIG. 27B). Levels of HTRA1 increase over time, even in the pCTM259 control sample. However, the relative increase in HTRA1 protein is significantly greater than background in cells treated with the AAV-HTRA1 plasmids driven by the BEST1_723, BEST1_340 and RPE65_146 promoters (FIG. 27A). After normalization to VEGF, there was a 4- to 6-fold increase in HtrA1 protein expression relative to the control plasmid that peaks at 24 hours and gradually decreases (FIG. 27C).

Together, these data demonstrate that HTRA1 expression at both the mRNA and protein levels can be upregulated by transient transfection of AAV2-HTRA1 plasmids driven by BEST1- or RPE65-based promoters.

14.12 CRISPR-Mediated Editing

This example shows experiments conducted to selectively delete a region of DNA on Chr10 encompassing the HTRA1 regulatory region in an allele-specific manner. Specifically, the CRISPR/Cas9 system and combinations of CRISPR guide RNA pairs were used to specifically remove a large section of DNA surrounding the rs10490924 SNP from either the risk or wild-type alleles in RPE1 cells.

Transfections were performed using the IDT Alt-R CRISPR system. The sequence of the individual guide RNAs are listed below in Table 13. The crRNAs were resuspended in Tris-EDTA solution to a final concentration of 200 μM. Each crRNA was combined with tracrRNA (IDT Cat. #1072532) in a 1:1 ratio in nuclease-free duplex buffer (IDT Cat. #11-01-03-01) and annealed by heating for 2 minutes at 95° C. and cooling gradually. The crRNA:tracrRNA complex was diluted with nuclease-free duplex buffer and spCas9-3NLS (IDT Cat. #1074181) was added to the complex. Samples were incubated for 20 minutes at room temperature to allow RNP formation. During the incubation, RPE1 cells were harvested and resuspended in electroporation buffer ‘R’ buffer at a concentration of 5×10⁷/ml. Cells were mixed with 1.8 μM of a 3′ and 5′ RNP complex along with 1.8 μM of carrier ssDNA. For each reaction, the final solution contains 2 μM spCas9-3NLS, 1.8 μM crRNA:tracrRNA and 1.8 μM carrier ssDNA. The cell-RNP mixture was electroporated using a 10 μl Neon pipette tip at 1300 volts, pulse width 20, and 2 pulses then transferred to 3 ml media in a 6-well plate. After 24 hours, the transfected cells were serially diluted to a concentration of 15 cells/ml. One hundred μl approximately 1.5 cells/well) was plated into 3×96 well plates for each sample.

TABLE 13
CRISPR Reagents Targeting the rs10490924 Region

| Short | | | | | | Efficiency | Specificity |
|---|---|---|---|---|---|---|---|
| Name | Name | Position | Strand | Sequence | PAM | Score | Score |
| A1 | ARMS2- | 122454810 | -1 | GAAGGACACA | AGG | 74.6 | 41.1 |
| | 1 | | | GGAACCACCG | | | |
| A2 | ARMS2- | 122454759 | 1 | CCCAGGACCG | AGG | 69.0 | 45.6 |
| | 2 | | | ATGGTAACTG | | | |
| WT1 | ARMS2- | 122457521 | 1 | GTCTAGCAGT | TGG | 71.5 | 73.6 |
| | WT- | | | GTCTACCCTG | | | |
| | 3UTR-1 | | | | | | |
| WT2 | ARMS2- | 122457627 | 1 | GGTTACGACC | GGG | 64.1 | 83.7 |
| | WT- | | | TCTGATGCTG | | | |
| | 3UTR-2 | | | | | | |
| INDEL1 | ARMS2- | 122457294 | -1 | TAATAACCGG | AGG | 62.8 | 47.0 |
| | INDEL- | | | GAGAGAAAGG | | | |
| | 1 | | | | | | |

Genomic DNA was purified from the clonal cultures using a DNeasy Blood & Tissue Kit (QIAGEN, Cat. #69504). PCR was performed using 200 nM of T7E1-Reg8-F (5′CTT ACCACCCTCGCTACATC3′) and INDEL-DEL-R1 (5′CCAGGGTGGTGTAATCC ATC3′) primers in Q5 PCR buffer (NEB, Cat. #B9027) containing 50 ng genomic DNA, 200 μM dNTPs (Thermo Fisher, Cat. #18427), and Q5 Hot Start High-Fidelity DNA Polymerase (NEB, Cat. #M0493). PCR products were visualized by agarose gel electrophoresis.

RPE1 cells are triploid for chromosome 10q26, containing two copies of the rs10490924 wild type allele and a single copy of the risk allele. PCR amplification of this region using untreated RPE1 cells yields two PCR products corresponding to a 3.5 kb band for the wild type alleles and a 3.2 kb band for the risk allele. Agarose gel electrophoresis shows a 2:1 bias in the density of the wild type vs the mutant allele. When cells are treated with CRISPR guide pairs specific for the wild type allele, there are three possible outcomes. A single wild type allele may be deleted, as indicated by a 1:1 ratio of the 3.5 kb/3.2 kb bands along with a 600-800 bp band generated from the cut allele with the fragment removed. If both of the wild type alleles are cut, the PCR will only yield a 3.2 kb band from the mutant allele and a 600-800 bp band from the cut wild type alleles. In contrast, cells treated with CRISPR guide pairs specific for the mutant allele should only show the denser 3.5 kb wild type band and a 600-800 bp band corresponding to the cut mutant allele.

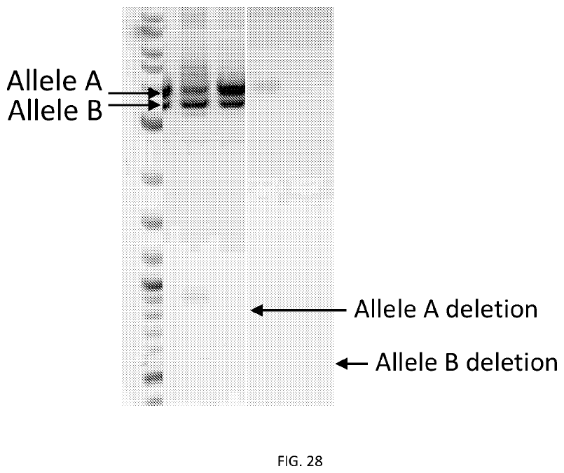

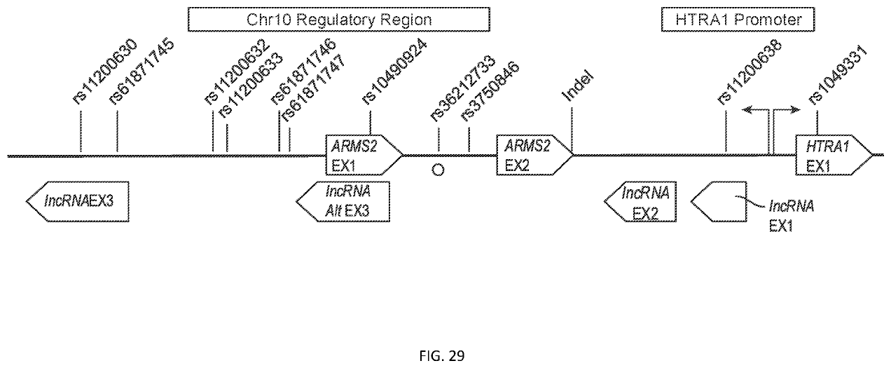

We determined that targeted pairs of crRNA guides could effectively remove a large section of the rs10490924 region in an allele-dependent manner (Table 14 and FIG. 28). This effect was demonstrated in a bulk population of transfected RPE1 cells for each CRISPR guide pair (data not shown). Based on those results, we isolated and established monoclonal cultures by limiting dilution of the bulk populations in order to accurately assess our ability to delete the 2 kb region. Genomic DNA from the monoclonal cultures was screened by PCR amplification of the rs10490924 region surrounding the cut site and analyzed by agarose gel electrophoresis. FIG. 28 shows an example in which the combination of the A2-WT2 crRNAs results in the deletion of the Chr10 non-risk allele (Allele A) while the combination of the A2-INDEL1 crRNAs results in the deletion of the Chr10 risk allele (Allele B). The other combinations of crRNAs generated the expected allele-specific deletion (data not shown). This demonstrates that we can specifically target the removal of a chromosomal region in an allele-specific manner.

TABLE 14
Combination of RNP Complexes for Removal of DNA Fragment from the rs10490924 Region.

| | | | | | CRISPR | | | CRISPR |
|---|---|---|---|---|---|---|---|---|
| | | | Uncut | | Cut PCR | Uncut | | Cut PCR |
| | | | PCR | | Product | PCR | | Product |
| | | | Product | | Size T7E1- | Product | | Size T7E1- |
| | | | Size | | Reg8-F + | Size | | Reg8-F + |
| | | | T7E1- | Fragment | INDEL- | T7E1- | Fragment | INDEL- |
| | 5′ | 3′ | Reg8-F + | Removed | DEL-R1 | Reg8-F + | Removed | DEL-R4 |
| | CRISPR | CRISPR | INDEL- | by | (Fragment | INDEL- | by | (Fragment |
| Template | Guide | Guide | DEL-R1 | CRISPR | Removed) | DEL-R4 | CRISPR | Removed) |
| WT | ARMS2- | ARMS2- | 3549 | 2711 | 838 | 3773 | 2711 | 1062 |
| | 1 | WT- | | | | | | |
| | | 3UTR-1 | | | | | | |
| | ARMS2- | ARMS2- | 3549 | 2762 | 787 | 3773 | 2762 | 1011 |
| | 2 | WT- | | | | | | |
| | | 3UTR-1 | | | | | | |
| | ARMS2- | ARMS2- | 3549 | 2817 | 732 | 3773 | 2817 | 956 |
| | 1 | WT- | | | | | | |
| | | 3UTR-2 | | | | | | |
| | ARMS2- | ARMS2- | 3549 | 2868 | 681 | 3773 | 2868 | 905 |
| | 2 | WT- | | | | | | |
| | | 3UTR-2 | | | | | | |
| Risk | ARMS2- | ARMS2- | 3160 | 2535 | 625 | 3384 | 2535 | 849 |
| | 2 | INDEL-1 | | | | | | |

TABLE 15
Nomenclature

| Short Name | Name | Deletion |
|---|---|---|
| A2 | ARMS2-2 | Allele A |
| WT2 | ARMS2-WT-3UTR-2 | 2868 bp del |
| A2 | ARMS2-2 | Allele B |
| INDEL1 | ARMS2-INDELK-1 | 2535 bp del |

### 15. References

Cabrera A C, Melo E, Roth D, Topp A, Delobel F, Stucki C, Chen C, Jakob P, Banfai B, Dunkley T, Schilling O, Huber S, Iacone R, Petrone P. (2017) HtrA1 activation is driven by an allosteric mechanism of intermonomer communication. BioRxiv doi:10.1101/163717.
Chan C, Shen D, Zhou M, Ross R, Ding X, Zhang K, Green R, Tuo J. (2007) Human htra1 in the archived eyes with age-related macular degeneration. Trans Am Ophthalmol Soc. 2007; 105: 92-98.
Chowers I, Meir T, Lederman M, Goldenberg-Cohen N, Cohen Y, Banin E, Averbukh E, Hemo I, Pollack A, Axer-Siegel R, Weinstein O, Hoh J, Zack D J, Galbinur T. (2008) Sequence variants in HTRA1 and LOC387715/ARMS2 and phenotype and response to photodynamic therapy in neovascular age-related macular degeneration in populations from Israel. Mol Vis 14:2263-71.
Colijn J M, Buitendijk G H S, Prokofyeva E. et al. (2017) Prevalence of age-related macular degeneration in Europe: The past and the future. Ophthalmology 124:1753-63.
Dewan A, Liu M, Hartman S, Zhang S S, Liu D T, Zhao C, Tam P O, Chan W M, Lam D S, Snyder M, Barnstable C, Pang C P, Hoh J. (2006) HTRA1 promoter polymorphism in wet age-related macular degeneration. Science: 314: 989-92.
Fisher S A, Abecasis G R, Yashar B M, et. al. (2005) Meta-analysis of genome scans of age-related macular degeneration. Hum Mol Genet 14: 2257-64.
Flaxman S R, Bourne R R A, Resnikoff S, et al. (2017) Global causes of blindness and distance vision impairment 1990-2020: a systematic review and meta-analysis. Lancet Glob Health 5(12): e1221-e1234. doi: 10.1016/S2214-109X(17)30393-5.
Fritsche L G, Loenhardt T, Janssen A, Fisher S A, Rivera A, Keilhauer C N, Weber B H. (2008) Age-related macular degeneration is associated with an unstable ARMS2 (LOC387715) mRNA. Nature Genetics 40: 892-6.
Fukutake, T. (2011) Cerebral Autosomal Recessive Arteriopathy with Subcortical Infarcts and Leukoencephalopathy (CARASIL): From Discovery to Gene Identification. J Stroke Cerebrovasc Dis 20:85-93.
Lonsdale J, Thomas J, Salvatore M et al. (2013) The Genotype-Tissue Expression (GTEx) project. Nat Genet 45: 580-5.
The GTEx Consortium (2015) The Genotype-Tissue Expression (GTEx) pilot analysis: Multitissue gene regulation in humans. Science 348, 648-660.
Grassman F, Heid I M, Weber B H, International AMD Genomics Consortium. (2017) Recombinant haplotypes narrow the ARMS/HTRA1 association signal for age-related macular degeneration. Genetics 205: 919-924.
Hara K, Shiga A, Fukutake T et al. (2009) Association of HTRA1 mutations and familial sschemic cerebral small-vessel disease. N Engl J Med 360: 1729-39.
Holz F G, Tadayoni R, Beatty S, Berger A, Cereda M G, Cortez R, Hoyng C B, Hykin P, Staurenghi G, Heldner S, Bogumil T, Heah T, Sivaprasad S. (2015) Multi-country real-life experience of anti-vascular endothelial growth factor therapy for wet age-related macular degeneration. Br J Ophthalmol 99(2):220-6.
Jones A, Kumar S, Zhang N, Tong Z, Yang J H, Watt C, Anderson J, Amrita, Fillerup H, McCloskey M, Luo L, Yang Z, Ambati B, Marc R, Oka C, Zhang K, Fu Y. (2011) Increased expression of multifunctional serine protease, HTRA1, in retinal pigment epithelium induces polypoidal choroidal vasculopathy in mice. Proc. Natl. Acad. Sci. U.S.A. August 30; 108(35): 14578-83.
Kanda A, Chen W, Othman M, Branham K E, Brooks M, Khanna R, He S, Lyons R, Abecasis G R, Swaroop A. (2007) A variant of mitochondrial protein LOC387715/ARMS2, not HTRA1, is strongly associated with age-related macular degeneration. PNAS 104:16227-32.
Kortvely E, Hauck S M, Duetsch G, Gloeckner C J, Kremmer E, Alge-Priglinger C S, Deeg C A, Ueffing M. (2010) ARMS2 is a constituent of the extracellular matrix providing a link between familial and sporadic age-related macular degenerations. Invest Ophtalmol Vis Sci 51: 79-88.
Lau C and Suh Y. (2017) In vivo genome editing in animals using AAV-CRISPR system: applications to translational research of human disease. (2017) F1000 Res. 2017; 6: 2153; doi: 10.12688/f1000research.11243.1.
Lambert S A, Jolma A, Campitelli L F, Das P K, Yin Y, Albu M, Chen X, Taipale J, Hughes T R, Weirauch M T (2018) Cell 172(4):650-665.
Moore D J and Clover G M. (2001) The effect of age on the macromolecular permeability of human Bruch's membrane. Invest Ophthalmol Vis Sci 42:2970-5.
Peng Y, Shekhar K, Yan W, Herrmann D, Sappington A, Bryman G S, vanZyl T, Do M T H, Regev A and Sanes J R. 2019 Molecular classification and comparative taxonomics of foveal and peripheral cells in primate retina. Cell 176:5:1222-1237.
Regillo C D, Busbee B G, Ho A C, Ding B, Haskova Z (2015) Baseline predictors of 12-month treatment response to ranibizumab in patients with wet age-related macular degeneration. Am J Ophthalmol 160(5):1014-23.
Rivera A, Fisher S A, Fritsche L G, Keilhauer C N, Lichtner P, Meitinger T, Weber B H. (2005) Hypothetical LOC387715 is a second major susceptibility gene for age-related macular degeneration, contributing independently of complement factor H to disease risk. Hum Mol Genet 14: 3227-36.
Vierkotten S, Muether P, Fauser S (2011) Overexpression of HTRA1 Leads to Ultrastructural Changes in the Elastic Layer of Bruch's Membrane via Cleavage of Extracellular Matrix Components. PLoS One. 2011; 6(8):e22959.
Wang N, Eckert K, Zomorrodi A, Xin P, Pan W, Shearer D, Weisz J, Maranus C, Clawson G, (2012) Plos I One, https://doi.org/10.1371/journal.pone.0039446.
Wang G, Scott W K, Whitehead P, Court B L, Kovach J L, Schwartz S G, Agarwal A, Dubovy S, Haines J L, Pericak-Vance M A. (2012) A novel ARMS2 splice variant is identified in human retina. Exp. Eye Res 94: 187-91.
Wong W L, Su X, Li X, Cheung C M, Klein R, Cheng C Y, Wong T Y (2014) Global prevalence of age-related macular degeneration and disease burden projection for 2020 and 2040: a systematic review and meta-analysis. Lancet Glob Health 2(2):e106-16. doi: 10.1016/52214-109X(13)70145-1.
Yang Z, Camp N J, Sun H, Tong Z, Gibbs D, Cameron D J, Chen H, Zhao Y, Pearson E, Li X, Chien J, Dewan A, Harmon J, Bernstein P S, Shridhar V, Zabriskie N A, Hoh J, Howes K, Zhang K. (2006) A variant of the HTRA1 gene increases susceptibility to age-related macular degeneration. Science 314: 992-3.
Zellner A, Scharrer E, Arzberger T et al. (2018) CADASIL brain vessels show a HTRA1 loss-of-function profile. Acta Neuropathol 136: 111-125.

TABLE 16
Summary of Sequences

| SEQ | Comment |
|---|---|
| 1 | Human HTRA1 nucleic acid sequence encoding the mature protein |
| 2 | Human HTRA1 protein (mature form) |
| 3 | Human HTRA1 nucleic acid sequence encoding pre-protein. |
| 4 | Human HTRA1 protein (precursor sequence). |
| 5 | HTRA1 Extended Promoter |
| 6 | VMD2 Promoter Fragment |
| 7 | HTRA1 Primary Promoter With 5′ UTR |
| 8 | HTRA1 Primary Promoter |
| 9 | sgRNA-P18 |
| 10 | LHX2 Motif |
| 11 | BEST1-723 Promoter Fragment |
| 12 | RPE65-V1 Promoter Fragment |
| 13 | HTRA1 Native Promoter (852 b) |
| 14 | 2 kb Region |
| 15-33 | sgRNA Guide Sequences |
| 34 | 4 kb region |
| 35 | Human codon-optimized HTRA1 nucleotide sequence |
| 36-49 | sgRNA Guide Sequences |
| 50 | A1(ARMS2-1) |
| 51 | A2(ARMS2-2) |
| 52 | INDEL1 |
| 53 | LHX2 Motif Long |
| 54 | Cas9 recognition sequence |
| 55 | MS2 binding sequence |
| 56 | MS2 polypeptide |
| 57-86 | Forward and Reverse Primers |
| 87-94 | Exemplary repair template sequences |

Although the foregoing invention has been described in some detail by way of illustration and example for purposes of clarity of understanding, one of skill in the art will appreciate that certain changes and modifications may be practiced within the scope of the appended claims.

The present invention may be embodied in other specific forms without departing from its structures, methods, or other essential characteristics as broadly described herein and claimed hereinafter. The described approaches are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates that may need to be independently confirmed.

1-44. (canceled)

45. A method of treating, preventing development of, slowing progression of, reversing or ameliorating symptoms and signs of age related macular degeneration (AMD) in a subject, comprising administering an agent that increases levels of HTRA1 mRNA and/or protein in an eye of the subject.

46. The method of claim 45 wherein administering the agent increases HTRA1 expression in the retinal pigment epithelium of the subject.

47. The method of claim 46 comprising delivering a polynucleotide comprising a sequence that encodes an exogenous HTRA1 protein to the eye, wherein a promoter is operably linked to the sequence that encodes the exogenous HTRA1 protein.

48. The method of claim 47 wherein the polynucleotide is delivered by a viral vector that is a retroviral vector, a lentivirus vector, a herpes virus vector, or an adeno-associated virus (AAV) vector.

49. The method of claim 48 wherein the viral vector is AAV1, AAV2, AAV3, AAV4, AAV5, AAV6, AAV7, AAV8, AAV9, AAV10, or AAV11.

50. The method of claim 47 wherein the polynucleotide comprises a human codon-optimized sequence encoding HTRA1.

51. The method of claim 47 wherein the promoter is an RPE specific promoter.

52. The method of claim 47 wherein the promoter is RPE65, BEST1, smCBA, sctmCBA, or VMD2 promoter.

53. The method of claim 45 comprising delivering an exogenous HTRA1 protein to the eye.

54. The method of claim 45 wherein administration of the agent results in an increase in transcription from an endogenous HTRA1 gene.

55. The method of claim 54 wherein the agent comprises an HTRA1 activating system, said system comprising a fusion protein of i) a DNA targeting protein domain capable of recognizing a target sequence within the HTRA1 transcriptional regulatory region, and ii) a transcriptional activator domain.

56. The method of claim 55 wherein the transcriptional activator domain binds an LHX2 binding motif.

57. The method of claim 54 wherein the agent comprises a CRISPRa activating system, said system comprising (a) a first vector comprising a DNA sequence encoding a fusion protein comprising an enzymatically inactive Cas protein domain (dCas) fused to a transcriptional activator domain, and (b) a second vector comprising a DNA sequence encoding a gRNA comprising at least 10 contiguous nucleotides corresponding to a sequence in the HTRA1 promoter [SEQ ID NO: 8] or in the HTRA1 2 kb regulatory region [SEQ ID NO:13], wherein the first and second vectors may be the same or different vectors.

58. The method of claim 57 wherein the enzymatically inactive Cas protein domain (dCas) is dCas9 or dCas12a.

59. The method claim 57 wherein the transcriptional activator domain binds an LHX2 binding motif.

60. The method according to claim 54 wherein the subject carries a risk allele in the HTRA1 gene enhancer region [SEQ ID NO:14] and the agent that results in an increase in transcription from an endogenous HTRA1 gene is a combination comprising (a) a ribonucleic acid complex comprising a guide RNA and a Cas protein and (ii) a template repair polynucleotide comprising a sequence of a non-risk allele corresponding to the risk allele.

61. The method of claim 60 wherein a risk allele is at rs36212733.

62. The method of claim 61 wherein the subject is homozygous for the risk allele

63. The method of claim 54, wherein the agent is administered by subretinal injection, suprachoroidal injection, transscleral injection or intravitreal injection.

64. The method of claim 45 wherein the subject does not exhibit clinical symptoms of chromosome 10-directed AMD.

65. The method of claim 45 wherein the subject is diagnosed with geographic atrophy or choroidal neovascularization (CNV)

66. The method of claim 45 wherein the subject
a) exhibits a Chromosome 10 AMD clinical phenotype; and/or
b) is homozygous or heterozygous for a chromosome 10 risk allele.

67. A ribonucleoprotein (RNP) complex comprising
a) guide RNA (gRNA) comprising a guide sequence of at least 10 contiguous nucleotides corresponding to a target sequence in the HTRA1 promoter or in the HTRA1 2 kb regulatory region and
b) a fusion protein comprising a CRISPR-associated protein (Cas) domain fused to a transcriptional activator domain,
wherein the Cas domain lacks nuclease activity.


SEQUENCE LISTING (94 sequences)

SEQ ID NO: 1 - DNA, 1374 bp, Homo sapiens
cagctgtcccgggccggccgctcggcgcctttggccgccgggtgcccagaccgctgcgagccggcgcgctgcccgccgcagccggagcactgcgagggcggccgggcccgggacgcgtgcggctgctgcgaggtgtgcggcgcgcccgagggcgccgcgtgcggcctgcaggagggcccgtgcggcgaggggctgcagtgcgtggtgcccttcggggtgccagcctcggccacggtgcggcggcgcgcgcaggccggcctctgtgtgtgcgccagcagcgagccggtgtgcggcagcgacgccaacacctacgccaacctgtgccagctgcgcgccgccagccgccgctccgagaggctgcaccggccgccggtcatcgtcctgcagcgcggagcctgcggccaagggcaggaagatcccaacagtttgcgccataaatataactttatcgcggacgtggtggagaagatcgcccctgccgtggttcatatcgaattgtttcgcaagcttccgttttctaaacgagaggtgccggtggctagtgggtctgggtttattgtgtcggaagatggactgatcgtgacaaatgcccacgtggtgaccaacaagcaccgggtcaaagttgagctgaagaacggtgccacttacgaagccaaaatcaaggatgtggatgagaaagcagacatcgcactcatcaaaattgaccaccagggcaagctgcctgtcctgctgcttggccgctcctcagagctgcggccgggagagttcgtggtcgccatcggaagcccgttttcccttcaaaacacagtcaccaccgggatcgtgagcaccacccagcgaggcggcaaagagctggggctccgcaactcagacatggactacatccagaccgacgccatcatcaactatggaaactcgggaggcccgttagtaaacctggacggtgaagtgattggaattaacactttgaaagtgacagctggaatctcctttgcaatcccatctgataagattaaaaagttcctcacggagtcccatgaccgacaggccaaaggaaaagccatcaccaagaagaagtatattggtatccgaatgatgtcactcacgtccagcaaagccaaagagctgaaggaccggcaccgggacttcccagacgtgatctcaggagcgtatataattgaagtaattcctgataccccagcagaagctggtggtctcaaggaaaacgacgtcataatcagcatcaatggacagtccgtggtctccgccaatgatgtcagcgacgtcattaaaagggaaagcaccctgaacatggtggtccgcaggggtaatgaagatatcatgatcacagtgattcccgaagaaattgaccca

SEQ ID NO: 2 - protein, 458 aa, Homo sapiens
QLSRAGRSAPLAAGCPDRCEPARCPPQPEHCEGGRARDACGCCEVCGAPEGAACGLQEGPCGEGLQCVVPFGVPASATVRRRAQAGLCVCASSEPVCGSDANTYANLCQLRAASRRSERLHRPPVIVLQRGACGQGQEDPNSLRHKYNFIADVVEKIAPAVVHIELFRKLPFSKREVPVASGSGFIVSEDGLIVTNAHVVTNKHRVKVELKNGATYEAKIKDVDEKADIALIKIDHQGKLPVLLLGRSSELRPGEFVVAIGSPFSLQNTVTTGIVSTTQRGGKELGLRNSDMDYIQTDAIINYGNSGGPLVNLDGEVIGINTLKVTAGISFAIPSDKIKKFLTESHDRQAKGKAITKKKYIGIRMMSLTSSKAKELKDRHRDFPDVISGAYIIEVIPDTPAEAGGLKENDVIISINGQSVVSANDVSDVIKRESTLNMVVRRGNEDIMITVIPEEIDP

SEQ ID NO: 3 - DNA, 1440 bp, Homo sapiens
atgcagatcccgcgcgccgctcttctcccgctgctgctgctgctgctggcggcgcccgcctcggcgcagctgtcccgggccggccgctcggcgcctttggccgccgggtgcccagaccgctgcgagccggcgcgctgcccgccgcagccggagcactgcgagggcggccgggcccgggacgcgtgcggctgctgcgaggtgtgcggcgcgcccgagggcgccgcgtgcggcctgcaggagggcccgtgcggcgaggggctgcagtgcgtggtgcccttcggggtgccagcctcggccacggtgcggcggcgcgcgcaggccggcctctgtgtgtgcgccagcagcgagccggtgtgcggcagcgacgccaacacctacgccaacctgtgccagctgcgcgccgccagccgccgctccgagaggctgcaccggccgccggtcatcgtcctgcagcgcggagcctgcggccaagggcaggaagatcccaacagtttgcgccataaatataactttatcgcggacgtggtggagaagatcgcccctgccgtggttcatatcgaattgtttcgcaagcttccgttttctaaacgagaggtgccggtggctagtgggtctgggtttattgtgtcggaagatggactgatcgtgacaaatgcccacgtggtgaccaacaagcaccgggtcaaagttgagctgaagaacggtgccacttacgaagccaaaatcaaggatgtggatgagaaagcagacatcgcactcatcaaaattgaccaccagggcaagctgcctgtcctgctgcttggccgctcctcagagctgcggccgggagagttcgtggtcgccatcggaagcccgttttcccttcaaaacacagtcaccaccgggatcgtgagcaccacccagcgaggcggcaaagagctggggctccgcaactcagacatggactacatccagaccgacgccatcatcaactatggaaactcgggaggcccgttagtaaacctggacggtgaagtgattggaattaacactttgaaagtgacagctggaatctcctttgcaatcccatctgataagattaaaaagttcctcacggagtcccatgaccgacaggccaaaggaaaagccatcaccaagaagaagtatattggtatccgaatgatgtcactcacgtccagcaaagccaaagagctgaaggaccggcaccgggacttcccagacgtgatctcaggagcgtatataattgaagtaattcctgataccccagcagaagctggtggtctcaaggaaaacgacgtcataatcagcatcaatggacagtccgtggtctccgccaatgatgtcagcgacgtcattaaaagggaaagcaccctgaacatggtggtccgcaggggtaatgaagatatcatgatcacagtgattcccgaagaaattgaccca

SEQ ID NO: 4 - protein, 480 aa, Homo sapiens
MQIPRAALLPLLLLLLAAPASAQLSRAGRSAPLAAGCPDRCEPARCPPQPEHCEGGRARDACGCCEVCGAPEGAACGLQEGPCGEGLQCVVPFGVPASATVRRRAQAGLCVCASSEPVCGSDANTYANLCQLRAASRRSERLHRPPVIVLQRGACGQGQEDPNSLRHKYNFIADVVEKIAPAVVHIELFRKLPFSKREVPVASGSGFIVSEDGLIVTNAHVVTNKHRVKVELKNGATYEAKIKDVDEKADIALIKIDHQGKLPVLLLGRSSELRPGEFVVAIGSPFSLQNTVTTGIVSTTQRGGKELGLRNSDMDYIQTDAIINYGNSGGPLVNLDGEVIGINTLKVTAGISFAIPSDKIKKFLTESHDRQAKGKAITKKKYIGIRMMSLTSSKAKELKDRHRDFPDVISGAYIIEVIPDTPAEAGGLKENDVIISINGQSVVSANDVSDVIKRESTLNMVVRRGNEDIMITVIPEEIDP

SEQ ID NO: 5 - DNA, 469 bp, Homo sapiens
gcgcgtgaccggggtccgcggtgccgcaacgccccgggtctgcgcagaggcccctgcagtccctgcccggcccagtccgagcttcccgggcgggcccccagtccggcgatttgcaggaactttccccggcgctcccacgcgaagccgccgcagggcccccttgcaaagttccattagtttgaaggacgcgaatctcagcgagagaacctgcggaaagcgaatatgtggggcgcgcagacggggaaactgagtcccgcgagagggccggcctgtgcgctgccccgcccgcgccccgccagcaccgccgtgcccgggcgcgcccgccctgccccctccgcgggcggtcccggtccagccgcccgccctccctcccgccatccggccagcccccatcccgggcgccgtgcccgtccccaaggcggctcgtcaccgctgcgaggccaatgggctgggccgcgcggccgcgcgc

SEQ ID NO: 6 - DNA, 796 bp, Artificial Sequence
  Description of Artificial Sequence Synthetic polynucleotide
caattctgtcattttactagggtgatgaaattcccaagcaacaccatccttttcagataagggcactgaggctgagagaggagctgaaacctacccggcgtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggaccaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccagaccggggatcctctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttac

SEQ ID NO: 7 - DNA, 400 bp, Homo sapiens
tccattagtttgaaggacgcgaatctcagcgagagaacctgcggaaagcgaatatgtggggcgcgcagacggggaaactgagtcccgcgagagggccggcctgtgcgctgccccgcccgcgccccgccagcaccgccgtgcccgggcgcgcccgccctgccccctccgcgggcggtcccggtccagccgcccgccctccctcccgccatccggccagcccccatcccgggcgccgtgcccgtccccaaggcggctcgtcaccgctgcgaggccaatgggctgggccgcgcggccgcgcgcactcgcacccgctgcccccgaggccctcctgcactctccccggcgccgctctccggccctcgccctgtccgccgccaccgccgccgccgccagagtcgcc

SEQ ID NO: 8 - DNA, 300 bp, Homo sapiens
tccattagtttgaaggacgcgaatctcagcgagagaacctgcggaaagcgaatatgtggggcgcgcagacggggaaactgagtcccgcgagagggccggcctgtgcgctgccccgcccgcgccccgccagcaccgccgtgcccgggcgcgcccgccctgccccctccgcgggcggtcccggtccagccgcccgccctccctcccgccatccggccagcccccatcccgggcgccgtgcccgtccccaaggcggctcgtcaccgctgcgaggccaatgggctgggccgcgcggccgcgcgc

SEQ ID NO: 9 - DNA, 102 bp, Artificial Sequence
  Description of Artificial Sequence Synthetic polynucleotide
cgccgtgcccgtccccaagggttttagagctagaaatagcaagttaaaataaggctagtccgttatcaacttgaaaaagtggcaccgagtcggtgctttttt

SEQ ID NO: 10 - DNA, 59 bp, Homo sapiens
gtggcgctttgtgcttgccatagtatatataaytagacaaatgagagaacacaaaggtt

SEQ ID NO: 11 - DNA, 723 bp, Artificial Sequence
  Description of Artificial Sequence Synthetic polynucleotide
ctctgaagcaacttactgatgggccctgccagccaatcacagccagaataacgtatgatgtcaccagcagccaatcagagctcctcgtcagcatatgcagaattctgtcattttactagggtgatgaaattcccaagcaacaccatccttttcagataagggcactgaggctgagagaggagctgaaacctacccggggtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccagacc

SEQ ID NO: 12 - DNA, 750 bp, Artificial Sequence
  Description of Artificial Sequence Synthetic polynucleotide
atactctcagagtgccaaacatataccaatggacaagaaggtgaggcagagagcagacaggcattagtgacaagcaaagatatgcagaatttcattctcagcaaatcaaaagtcctcaacctggttggaagaatattggcactgaatggtatcaataaggttgctagagagggttagaggtgcacaatgtgcttccataacattttatacttctccaatcttagcactaatcaaacatggttgaatactttgtttactataactcttacagagttataagatctgtgaagacagggacagggacaatacccatctctgtctggttcataggtggtatgtaatagatatttttaaaaataagtgagttaatgaatgagggtgagaatgaaggcacagaggtattagggggaggtgggccccagagaatggtgccaaggtccagtggggtgactgggatcagctcaggcctgacgctggccactcccacctagctcctttctttctaatctgttctcattctccttgggaaggattgaggtctctggaaaacagccaaacaactgttatgggaacagcaagcccaaataaagccaagcatcagggggatctgagagctgaaagcaacttctgttccccctccctcagctgaaggggtggggaagggctcccaaagccataactccttttaagggatttagaaggcataaaaaggcccctggctgagaacttccttcttcattctgcagttgg

SEQ ID NO: 13 - DNA, 853 bp, Homo sapiens
gtcccaacggatgcaccaaagattctccagtgggaaatcaaatttttgataacaagtgttttgaaataatcacgcacttggtgaaaaatcaaaagaggggaaaacccctttcccatctgagaccgctccaccctcgccagttacgagctgccgagccgcttcctaggctctctgcgaatacggacacgcatgccacccacaacaactttttaaaagaatcagacgtgtgaaggattctattcgaattacttctgctctctgcttttatcacttcactgtgggtctgggcgcgggctttctgccagctccgcggacgctgccttcgtccggccgcagaggccccgcggtcagggtcccgcgtgcggggtaccgggggcagaaccagcgcgtgaccggggtccgcggtgccgcaacgccccgggtctgcgcagaggcccctgcagtccctgcccggcccagtccgagcttcccgggcgggcccccagtccggcgatttgcaggaactttccccggcgctcccacgcgaagccgccgcagggcccccttgcaaagttccattagtttgaaggacgcgaatctcagcgagagaacctgcggaaagcgaatatgtggggcgcgcagacggggaaactgagtcccgcgagagggccggcctgtgcgctgccccgcccgcgccccgccagcaccgccgtgcccgggcgcgcccgccctgccccctccgcgggcggtcccggtccagccgcccgccctccctcccgccatccggccagcccccatcccgggcgccgtgcccgtccccaaggcggctcgtcaccgctgcgaggccaatgggctgggccgcgcggccgcgcgc

SEQ ID NO: 14 - DNA, 2056 bp, Homo sapiens
gatactagggacctctgttgcctcctctggcagagcaggactgaggggtggaccctccctgagaccacccaacaattcagggtggagttatcagggcgccctgactcctgggggcatttttgtgtgacgggaaaagacaatgctcctggctgagtgagatggcagctggcttggcaaggggacagcacctttgtcaccacattatgtccctgtaccctacatgctgcgcctatacccaggaccgatggtaactgaggcggaggggaaaggagggcctgagatggcaagtctgtcctcctcggtggttcctgtgtccttcatttccactctgcgagagtctgtgctggaccctggagttggtggagaaggagccagtgacaagcagaggagcaaactgtctttatcacactccatgatcccagctgctaaaatccacactgagctctgcttaccagccttcttctctcctgctggaacccagaggaggttccagcagcctcagcaccacctgacactggtaagaaatgcagatgatcaggccttaccccagacctattgaatcagaaattctggagtggtgccctgcagcttgcattttaaccagccttcaggtgcttctgatgcatgctcaggtttgagcaccactggccacagggaggcctaggcaattcagccttcctctggttgaatagctggagaattgggaatatcagtaaatacttccaatgcacctgctacatgccagaaaaaggaaacaagaagacgcagtaggtctgagaaagtgatggggtgagcagaaacccaaagcttatagaaggccatctgagtggcccctcaagccggtgaattggctttagggtttactgaaggaggtggaaacctcagcctgcttctcgtccgggttgttagaggagtcatttagaaagctgtaccattctttcaatattctcacggctttccagtgctcatttttcctgctcatttatggattaaaaaaaatccttggctgtatgtgtaagaaaacaacaatgcaagtttgtagagaaagaatctgggccttacaggtcacgttggtttaaaatttagacatcaagcagcttagagaccatgttgccaaataagcttagtaaatgctttctaatgcttacggaactgtggcgctttgtgcttgccatagtatatataattagacaaatgagagaacacaaaggttgaaccccttccctctcttaatttttgttttttacaagcagatttaaaattctggctcataatgtccttgattcaatgttaaaccattttgcctaaatggcagcatgttctaaatgtgagcgcgctcagcttttcaagctgctcccgagtgacagaaattgacaagctgtcattcaagacctttcggtggctgcctggggctctgtttgaattgtatctgtctgatactttaccatggagagtgaaaaatttgatcacatgccatgcttttaattttctaaagcaaatatgttggaaggagccaattaatgcaaagatggactgctggtctcatgcaactgatttaggggaagggttcgcctaaattaataaaagatctgaattatagatcttaacaaatacatagaatgtaaaggcttaaaaggaaactgagcagcagcaggcctggggttggcttttaagtatctatatttaactaatagacatgaatgttttgatttgatattagaaatgctagtgctggagtctctgagcctactctggctcgagaggatgccctatctaaaaaacaaaaaacaaaaaaaaaaaagaaaaaagaaaaaaagaaatgctggtattgtaattctaaagtgcttcagaaattctcaaaaataggccaggcatggtggctcatggctgtataccagcactttgggaggccaaggtgggcaaatcacttgcagtcaggagtttgagaccagcctggtcaacatggtgaaaccccatctctactaaaaatacagaaattagccaggcatggtggcagcacctgtaatcccagctacttgggaggctgaggcaggag

SEQ ID NO: 15 - DNA, 20 bp, Artificial Sequence
  Description of Artificial Sequence Synthetic oligonucleotide
cacgcgggaccctgaccgcg

SEQ ID NO: 16 - DNA, 20 bp, Artificial Sequence
  Description of Artificial Sequence Synthetic oligonucleotide
tgcccccggtaccccgcacg

SEQ ID NO: 17 - DNA, 20 bp, Artificial Sequence
  Description of Artificial Sequence Synthetic oligonucleotide
cccggggcgttgcggcaccg

SEQ ID NO: 18 - DNA, 20 bp, Artificial Sequence
  Description of Artificial Sequence Synthetic oligonucleotide
cctgcaaatcgccggactgg

SEQ ID NO: 19 - DNA, 20 bp, Artificial Sequence
  Description of Artificial Sequence Synthetic oligonucleotide
ctgcggaaagcgaatatgtg

SEQ ID NO: 20 - DNA, 20 bp, Artificial Sequence
  Description of Artificial Sequence Synthetic oligonucleotide
ggaaactgagtcccgcgaga

SEQ ID NO: 21 - DNA, 20 bp, Artificial Sequence
  Description of Artificial Sequence Synthetic oligonucleotide
gggctggccggatggcggga

SEQ ID NO: 22 - DNA, 20 bp, Artificial Sequence
  Description of Artificial Sequence Synthetic oligonucleotide
agcggtgacgagccgccttg

SEQ ID NO: 23 - DNA, 20 bp, Artificial Sequence
  Description of Artificial Sequence Synthetic oligonucleotide
actgagtcccgcgagagggc

SEQ ID NO: 24 - DNA, 20 bp, Artificial Sequence
  Description of Artificial Sequence Synthetic oligonucleotide
cgcacaggccggccctctcg

SEQ ID NO: 25 - DNA, 20 bp, Artificial Sequence
  Description of Artificial Sequence Synthetic oligonucleotide
gcgcgggcggggcagcgcac

SEQ ID NO: 26 - DNA, 20 bp, Artificial Sequence
  Description of Artificial Sequence Synthetic oligonucleotide
cccgccagcaccgccgtgcc

SEQ ID NO: 27 - DNA, 20 bp, Artificial Sequence
  Description of Artificial Sequence Synthetic oligonucleotide
agggcgggcgcgcccgggca

SEQ ID NO: 28 - DNA, 20 bp, Artificial Sequence
  Description of Artificial Sequence Synthetic oligonucleotide
cgccctgccccctccgcggg

SEQ ID NO: 29 - DNA, 20 bp, Artificial Sequence
  Description of Artificial Sequence Synthetic oligonucleotide
ggaccgggaccgcccgcgga

SEQ ID NO: 30 - DNA, 20 bp, Artificial Sequence
  Description of Artificial Sequence Synthetic oligonucleotide
ggagggcgggcggctggacc

SEQ ID NO: 31 - DNA, 20 bp, Artificial Sequence
  Description of Artificial Sequence Synthetic oligonucleotide
gggcacggcgcccgggatgg

SEQ ID NO: 32 - DNA, 20 bp, Artificial Sequence
  Description of Artificial Sequence Synthetic oligonucleotide
cgccgtgcccgtccccaagg

SEQ ID NO: 33 - DNA, 20 bp, Artificial Sequence
  Description of Artificial Sequence Synthetic oligonucleotide
gtcaccgctgcgaggccaat

SEQ ID NO: 34 - DNA, 4090 bp, Artificial Sequence
  Description of Artificial Sequence Synthetic polynucleotide
tttaagcaattcttttgcctcagcctctggaattacaggcacgcgccaccatgccctgctaatttttgtatttttagtagagacagggttccaccatgttggccaggctgatctggaactcctgacctcaagtgatctgcccgcctcggcctcccaaagtgctgggattacaggtgtgagccaccgcacctggccaactattttaaaaaattttaagagactgagtctcactctgtcacccagtctggtgcagtggcacgatcatggcttcagccttaaagtcctgggctcaagcaatcctcccacctcagccccccgagtagctgggaatacaagtgtgcacagtcacactaaactaaaaaaaaaaaaaaaaactaattttattttattttattttattttttatttatttattttttgtagggatgggtttttgctatgttgcctaggctggtctcaaagtcctggcctcaagcaatcctcctgcttcatcctcccaaagtgctgtgattacaggcatgagccactgttcccagcctctccccactcttctagccctaagcaagcacaaatctattttctgtatctgtagattttcctgttatacacatttcttatgaatgaaatcatataatatgttgtcttttgtatctggcttctttcatttagccccatgctttcagggttcaccatgttgtagtgggtattttattcttttttatggctgaatagtatttcattttatggatatgccataatttatctgttatcagttgatggacatttgggttgtttctacctttgggctgttatgaatagtgctgctatgaacatgtgtgttccagtttctgtatggacacatgttttcctttctcttgggtagctacctaggaatggaaatgctggatcatatattcactctgtttgaagaactgccagactgttttccaaagcagctataccattttacaatcccactagcagtgcatgaggattctgatttctccacatccttgctgatacttgttatcatctgactttttgattctggctaccttagtgcctatgaagtagtatctcaatgtggttttgatttgttgatttgctgatgactagagatgccaagcatcttctcatgtgtttatttgtgctcttagagtttttaattcagttggtctggaatagttttttttttccttttattttttatttttttgagacagagtcttgctctgtcaccaagctggagtgcagtggcgtgatcttggctcactgcaacctctgactccctggttcgggctattctcctgcctcagcctcccgagtaactgggattacaggcacgcaccaccatgcccagctaatttttgtatttttagtagagccaggattccaccatgttagccatgatggtctcgatctcctgagctcgtgatctgtccaccttggcttcccaaagtggtgggatgacaggcgtgagccatggcgcccggctttttttttccttttaaaaaagtgtttggctgggagaagtggttcacacctgtaatcccaacactttgggaggccgaggcaggaggatcacttgagcccaggacttcaagacctgcctgggcaacatagtaagaccccatctccaaaaaaaaaaaaaaaaagtaaattagccaggcgtggtgctgcacacctgtggtcccagctacttgggaggctgaggtgggaggatcacctgagccgaggagtatgaggctgcagtgagccatgatcatgccactggattccagcctgggtgacaaagtgagaccctgtctcagaaaaaaaaaaaaaggctggggggcctgggctaggattatgcctgtaatcctagcacttttgcaggtagattgcttgagctcggagttcgataccagcctggcaacatggcaaaacctggtctctacaaaaaacataaaaaatagctgggcgtggtctgtagctgtagtcccagctactcaggtggctgaggtaggaggatcaccttagcccgaggatgttgaggccacaataagctgtggtcataccactgcacaccagcctggacaacagaatgagaccccatctcaaaaaaaaaagttttttattttgaaataattttaggtttagagaaaagttgaaaaatagtaaaacaacaacaacaacaaaaaaacaacaaaaaatcccaaaacccccaaaactctcattgacccttatctcagatttcccgaatgcttaccaccctcgctacatcattcaagttcttggaaacatttaaagcatgtgaaacatttaaaacatttaagttggaggctttaagttgcacgtcctttatttctaaatatttcagtgtgtttttcttaaaaaaaattttctcataccacagtaacatgatcaaaattggaaaatcaacattgattcaatactatgatctacaatcaaggttttttttttttttcaaatccctgggtctctgcattttttaaaagcttcacagatgatttcaatggatactagggacctctgttgcctcctctggcagagcaggactgaggggtggaccctccctgagaccacccaacaattcagggtggagttatcagggcgccctgactcctgggggcatttttgtgtgacgggaaaagacaatgctcctggctgagtgagatggcagctggcttggcaaggggacagcacctttgtcaccacattatgtccctgtaccctacatgctgcgcctatacccaggaccgatggtaactgaggcggaggggaaaggagggcctgagatggcaagtctgtcctcctcggtggttcctgtgtccttcatttccactctgcgagagtctgtgctggaccctggagttggtggagaaggagccagtgacaagcagaggagcaaactgtctttatcacactccatgatcccagctgctaaaatccacactgagctctgcttaccagccttcttctctcctgctggaacccagaggaggttccagcagcctcagcaccacctgacactggtaagaaatgcagatgatcaggccttaccccagacctattgaatcagaaattctggagtggtgccctgcagcttgcattttaaccagccttcaggtgcttctgatgcatgctcaggtttgagcaccactggccacagggaggcctaggcaattcagccttcctctggttgaatagctggagaattgggaatatcagtaaatacttccaatgcacctgctacatgccagaaaaaggaaacaagaagacgcagtaggtctgagaaagtgatggggtgagcagaaacccaaagcttatagaaggccatctgagtggcccctcaagccggtgaattggctttagggtttactgaaggaggtggaaacctcagcctgcttctcgtccgggttgttagaggagtcatttagaaagctgtaccattctttcaatattctcacggctttccagtgctcatttttcctgctcatttatggattaaaaaaaatgccttggctgtatgtgtaagaaaacaacaatgcaagtttgtagagaaagaatctgggccttacaggtcacgttggtttaaaatttagacatcaagcagcttagagaccatgttgccaaataagcttagtaaatgctttctaatgcttacggaactgtggcgctttgtgcttgccatagtatatataattagacaaatgagagaacacaaaggttgaaccccttccctctcttaatttttgttttttacaagcagatttaaaattctggctcataatgtccttgattcaatgttaaaccattttgcctaaatggcagcatgttctaaatgtgagcgcgctcagcttttcaagctgctcccgagtgacagaaattgacaagctgtcattcaagacctttcggtggctgcctggggctctgtttgaattgtatctgtctgatactttaccatggagagtgaaaaatttgatcacatgccatgcttttaattttctaaagcaaatatgttggaaggagccaattaatgcaaagatggactgctggtctcatgcaactgatttaggggaagggttcg

SEQ ID NO: 35 - DNA, 1443 bp, Artificial Sequence
  Description of Artificial Sequence Synthetic polynucleotide
atgcagattcctagagctgctctgctgcccctgctgctgttgcttcttgctgctcctgctagcgcccagctgtccagagctggaagatctgctcctctggccgctggctgtcctgatagatgcgaacctgccagatgtcctcctcagcctgagcactgtgaaggcggcagagccagagatgcctgcggctgttgtgaagtgtgcggagcacctgaaggcgccgcttgtggacttcaagaaggaccttgcggagagggcctgcagtgcgttgtgccttttggagtgcctgcctctgccacagttaggcggagagcacaagctggcctgtgtgtgtgcgcctcttctgagccagtgtgtggctccgacgccaacacctacgccaatctgtgtcagctgagagccgccagcagaagaagcgagagactgcacagacctccagtgatcgtgctgcagagaggcgcttgcggacagggacaagaggaccccaatagcctgcggcacaagtacaactttatcgccgacgtggtggaaaagatcgcccctgccgtggtgcacatcgagctgttcagaaagcttccattcagcaagcgcgaggtgccagtggcctctggatctggctttatcgtgtccgaggacggcctgatcgtgacaaatgcccacgtggtcaccaacaagcacagagtgaaggtggaactgaagaacggcgccacctacgaggccaagatcaaggacgtggacgagaaggccgacattgccctgatcaagatcgaccaccagggcaagctgccagtgctgctgcttggcagaagcagcgaactcagacctggcgagtttgtggtggccatcggaagccctttcagcctgcaaaacaccgtgaccaccggcatcgtgtccaccacacaaagaggcggcaaagagctgggcctgagaaacagcgacatggactacatccagaccgacgccatcatcaactacggcaactctggcggccctctggtcaacctggatggcgaagtgatcggcatcaacaccctgaaagtgacagccggcatcagcttcgctatccccagcgataagatcaagaagttcctgaccgagagccacgaccggcaggccaagggaaaagccatcaccaagaagaagtacatcggaatccggatgatgagcctgaccagcagcaaggccaaagaactgaaggaccggcacagagacttccccgatgtgatctctggcgcctacatcattgaagtgatccccgacacaccagccgaagccggcggactgaaagaaaacgacgtgatcatcagcatcaacggccagagcgtggtgtccgccaacgatgtgtctgatgtgatcaagcgggaaagcaccctgaacatggttgtgcggaggggcaacgaggatatcatgatcaccgtgattcccgaggaaatcgacccctga

SEQ ID NO: 36 - DNA, 20 bp, Artificial Sequence
  Description of Artificial Sequence Synthetic oligonucleotide
ctggtaagcagagctcagtg

SEQ ID NO: 37 - DNA, 20 bp, Artificial Sequence
  Description of Artificial Sequence Synthetic oligonucleotide
aaggagccagtgacaagcag

SEQ ID NO: 38 - DNA, 20 bp, Artificial Sequence
  Description of Artificial Sequence Synthetic oligonucleotide
tggttaaaatgcaagctgca

SEQ ID NO: 39 - DNA, 20 bp, Artificial Sequence
  Description of Artificial Sequence Synthetic oligonucleotide
ttgaatcagaaattctggag

SEQ ID NO: 40 - DNA, 20 bp, Artificial Sequence
  Description of Artificial Sequence Synthetic oligonucleotide
tgagcaggaaaaatgagcac

SEQ ID NO: 41 - DNA, 20 bp, Artificial Sequence
  Description of Artificial Sequence Synthetic oligonucleotide
ccgtgagaatattgaaagaa

SEQ ID NO: 42 - DNA, 20 bp, Artificial Sequence
  Description of Artificial Sequence Synthetic oligonucleotide
ttgtctagttatatatacta

SEQ ID NO: 43 - DNA, 20 bp, Artificial Sequence
  Description of Artificial Sequence Synthetic oligonucleotide
agacaaatgagagaacacaa

SEQ ID NO: 44 - DNA, 20 bp, Artificial Sequence
  Description of Artificial Sequence Synthetic oligonucleotide
aatggtttaccattgaatca

SEQ ID NO: 45 - DNA, 20 bp, Artificial Sequence
  Description of Artificial Sequence Synthetic oligonucleotide
gcttgtcaatttctgtcact

SEQ ID NO: 46 - DNA, 20 bp, Artificial Sequence
  Description of Artificial Sequence Synthetic oligonucleotide
agccccaggcagccaccaaa

SEQ ID NO: 47 - DNA, 20 bp, Artificial Sequence
  Description of Artificial Sequence Synthetic oligonucleotide
tacaattcaaacagagcccc

SEQ ID NO: 48 - DNA, 20 bp, Artificial Sequence
  Description of Artificial Sequence Synthetic oligonucleotide
gcctcatgcaactgatttag

SEQ ID NO: 49 - DNA, 20 bp, Artificial Sequence
  Description of Artificial Sequence Synthetic oligonucleotide
ttaatgcaaagatggactgc

SEQ ID NO: 50 - DNA, 20 bp, Homo sapiens
gaaggacacaggaaccaccg

SEQ ID NO: 51 - DNA, 20 bp, Homo sapiens
cccaggaccgatggtaactg

SEQ ID NO: 52 - DNA, 20 bp, Artificial Sequence
  Description of Artificial Sequence Synthetic oligonucleotide
taataaccgggagagaaagg

SEQ ID NO: 53 - DNA, 28 bp, Homo sapiens
ttgccatagtatatataaytagacaaat

SEQ ID NO: 54 - RNA, 81 nt, Artificial Sequence
  Description of Artificial Sequence Synthetic oligonucleotide
guuuuagagcuagaaauagcaaguuaaaauaaggcuaguccguuaucaacuugaaaaaguggcaccgagucggugcuuuuu

SEQ ID NO: 55 - RNA, 26 nt, Artificial Sequence
  Description of Artificial Sequence Synthetic oligonucleotide
aacaugaggaucacccaugucugcag

SEQ ID NO: 56 - protein, 130 aa, Artificial Sequence
  Description of Artificial Sequence Synthetic polypeptide
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 57 - DNA, 27 bp, Artificial Sequence
  Description of Artificial Sequence Synthetic primer
cggggtaccccctgccagccaatcaca

SEQ ID NO: 58 - DNA, 32 bp, Artificial Sequence
  Description of Artificial Sequence Synthetic primer
cgcggatccaaatccagaggctaaaggatctg

SEQ ID NO: 59 - DNA, 30 bp, Artificial Sequence
  Description of Artificial Sequence Synthetic primer
cggggtaccctctgaagcaacttactgatg

SEQ ID NO: 60 - DNA, 26 bp, Artificial Sequence
  Description of Artificial Sequence Synthetic primer
cgcggatccggtctggcgactaggct

SEQ ID NO: 61 - DNA, 26 bp, Artificial Sequence
  Description of Artificial Sequence Synthetic primer
cggggtacccctgccagccaatcaca

SEQ ID NO: 62 - DNA, 31 bp, Artificial Sequence
  Description of Artificial Sequence Synthetic primer
cgcggatccctgtgctgagcttcaacttctg

SEQ ID NO: 63 - DNA, 26 bp, Artificial Sequence
  Description of Artificial Sequence Synthetic primer
cggggtacccctgccagccaatcaca

SEQ ID NO: 64 - DNA, 26 bp, Artificial Sequence
  Description of Artificial Sequence Synthetic primer
cgcggatccggtctggcgactaggct

SEQ ID NO: 65 - DNA, 29 bp, Artificial Sequence
  Description of Artificial Sequence Synthetic primer
cggggtacccaatcagagctcctcgtcag

SEQ ID NO: 66 - DNA, 31 bp, Artificial Sequence; identical to SEQ ID NO: 62
  Description of Artificial Sequence Synthetic primer

SEQ ID NO: 67 - DNA, 28 bp, Artificial Sequence
  Description of Artificial Sequence Synthetic primer
cggggtaccgaacactggtggagcagat

SEQ ID NO: 68 - DNA, 27 bp, Artificial Sequence
  Description of Artificial Sequence Synthetic primer
cgcggatccactccctgggactctgtg

SEQ ID NO: 69 - DNA, 31 bp, Artificial Sequence
  Description of Artificial Sequence Synthetic primer
cggggtaccgaaattcccaagcaacaccatc

SEQ ID NO: 70 - DNA, 29 bp, Artificial Sequence
  Description of Artificial Sequence Synthetic primer
cgcggatccttcttcttggagggtgttgg

SEQ ID NO: 71 - DNA, 35 bp, Artificial Sequence
  Description of Artificial Sequence Synthetic primer
cggggtaccctggttcataggtggtatgtaataga

SEQ ID NO: 72 - DNA, 27 bp, Artificial Sequence
  Description of Artificial Sequence Synthetic primer
cgcggatccagagaatggtgccaaggt

SEQ ID NO: 73 - DNA, 31 bp, Artificial Sequence
  Description of Artificial Sequence Synthetic primer
cggggtaccatactctcagagtgccaaacat

SEQ ID NO: 74 - DNA, 32 bp, Artificial Sequence
  Description of Artificial Sequence Synthetic primer
cgcggatccccaactgcagaatgaagaaggaa

SEQ ID NO: 75 - DNA, 29 bp, Artificial Sequence
  Description of Artificial Sequence Synthetic primer
cggggtaccgagggttagaggtgcacaat

SEQ ID NO: 76 - DNA, 30 bp, Artificial Sequence
  Description of Artificial Sequence Synthetic primer
cgcggatccaaaggagttatggctttggga

SEQ ID NO: 77 - DNA, 32 bp, Artificial Sequence
  Description of Artificial Sequence Synthetic primer
cggggtaccgagaatgaaggcacagaggtatt

SEQ ID NO: 78 - DNA, 34 bp, Artificial Sequence
  Description of Artificial Sequence Synthetic primer
cgcggatccccaaggagaatgagaacagattaga

SEQ ID NO: 79 - DNA, 31 bp, Artificial Sequence
  Description of Artificial Sequence Synthetic primer
cggggtaccaacctggttggaagaatattgg

SEQ ID NO: 80 - DNA, 34 bp, Artificial Sequence; identical to SEQ ID NO: 78
  Description of Artificial Sequence Synthetic primer

SEQ ID NO: 81 - DNA, 32 bp, Artificial Sequence; identical to SEQ ID NO: 77
  Description of Artificial Sequence Synthetic primer

SEQ ID NO: 82 - DNA, 32 bp, Artificial Sequence; identical to SEQ ID NO: 74
  Description of Artificial Sequence Synthetic primer

SEQ ID NO: 83 - DNA, 27 bp, Artificial Sequence
  Description of Artificial Sequence Synthetic primer
cggggtaccagagaatggtgccaaggt

SEQ ID NO: 84 - DNA, 32 bp, Artificial Sequence; identical to SEQ ID NO: 74
  Description of Artificial Sequence Synthetic primer

SEQ ID NO: 85 - DNA, 29 bp, Artificial Sequence
  Description of Artificial Sequence Synthetic primer
cggggtaccgagggttagaggtgcacaat

SEQ ID NO: 86 - DNA, 34 bp, Artificial Sequence; identical to SEQ ID NO: 78
  Description of Artificial Sequence Synthetic primer

SEQ ID NO: 87 - DNA, 21 bp, Homo sapiens
gatcccagctgctaaaatcca

SEQ ID NO: 88 - DNA, 22 bp, Homo sapiens
attctggagtggtgccctgcag

SEQ ID NO: 89 - DNA, 21 bp, Homo sapiens
atattctcacggctttccagt

SEQ ID NO: 90 - DNA, 21 bp, Homo sapiens
tgtgcttgccatagtatatat

SEQ ID NO: 91 - DNA, 21 bp, Homo sapiens
gtatatataattagacaaatg

SEQ ID NO: 92 - DNA, 21 bp, Homo sapiens
tgattcaatgttaaaccattt

SEQ ID NO: 93 - DNA, 21 bp, Homo sapiens
caagacctttcggtggctgcc

SEQ ID NO: 94 - DNA, 21 bp, Homo sapiens
ggactgctggtctcatgcaac